(19) United States
(12) Patent Application Publication
(10) Pub. No.: US 2024/0156999 A1
(43) Pub. Date: May 16, 2024

(54) MULTIMERIC CHELATOR COMPOUNDS FOR USE IN TARGETED RADIOTHERAPY

(71) Applicants: Bayer Aktiengesellschaft, Leverkusen (DE); Bayer AS, Oslo (NO)

(72) Inventors: Thomas BRUMBY, Berlin (DE); Alan CUTHBERTSON, Oslo (NO); Bård INDREVOLL, Oslo (NO); Waqas RAFIQUE, Oslo (NO); Vilde Roko KROGSTIE, Oslo (NO); Véronique CRUCIANI, Oslo (NO); Alexander KRISTIAN, Oslo (NO)

(73) Assignees: Bayer Aktiengesellschaft, Leverkusen (DE); Bayer AS, Oslo (NO)

(21) Appl. No.: 18/275,375

(22) PCT Filed: Jan. 31, 2022
(86) PCT No.: PCT/EP2022/052170

§ 371 Date: Aug. 1, 2023

(30) Foreign Application Priority Data
Feb. 1, 2021 (EP) 21154574.4

(51) Int. Cl. A61K 51/10; A61K 51/04; A61P 35/00; C07D 413/14

(52) U.S. Cl. CPC A61K 51/1096; A61K 51/0482; A61P 35/00; C07D 413/14

(57) ABSTRACT

The present invention covers compounds of general formula (I): [(C)n-L]-(V)m (I) where C is a chelator and n>1, L is a multi-functional linker moiety comprising multiple functional groups for the covalent attachment of chelator such as a polyamine or polyacid-containing backbone or amino acid containing polymer comprising side-chains with amino, thiol or carboxylic acid moieties such as lysine, cysteine or glutamic acid and V is a tissue targeting moiety where m=1-5 which preferentially coupled through a coupling moiety to either the multifunctional linker moiety L or directly to the chelator moiety C, and stereoisomers, tautomers, N-oxides, hydrates, solvates, and salts thereof, and mixtures of same.

The present invention relates to new chelating agents for alpha-particle emitting radionuclides, as described and defined herein, methods of preparing said compounds, intermediate compounds useful for preparing said compounds, pharmaceutical compositions and combinations comprising said compounds, and the use of said compounds for manufacturing pharmaceutical compositions for the treatment or prophylaxis of diseases, in particular of hyperplastic or neoplastic disorders, as a sole agent or in combination with other active ingredients.

### BACKGROUND

Specific cell killing can be essential for the successful treatment of a variety of diseases in mammalian subjects. Typical examples of this are in the treatment of malignant diseases such as sarcomas and carcinomas. However the selective elimination of certain cell types can also play a key role in the treatment of other diseases, especially hyperplastic and neoplastic diseases.

The most common methods of selective treatment are currently surgery, chemotherapy and external beam irradiation. Targeted radionuclide therapy is, however, a promising and developing area with the potential to deliver highly cytotoxic radiation specifically to cell types associated with disease. The most common forms of radiopharmaceuticals currently authorised for use in humans employ beta-emitting and/or gamma-emitting radionuclides. There has, however, been some interest in the use of alpha-emitting radionuclides in therapy because of their potential for more specific cell killing. The radiation range of typical alpha emitters in physiological surroundings is generally less than 100 micrometres, the equivalent of only a few cell diameters. This makes these sources well suited for the treatment of tumours, including micrometastases, because they have the range to reach neighbouring cells within a tumour but if they are well targeted then little of the radiated energy will pass beyond the target cells. Thus, not every cell need be targeted but damage to surrounding healthy tissue may be minimised (see Feinendegen et al., Radiat Res 148:195-201 (1997)). In contrast, a beta particle has a range of 1 mm or more in water (see Wilbur, Antibody Immunocon Radiopharm 4: 85-96 (1991)).

The energy of alpha-particle radiation is high in comparison with that carried by beta particles, gamma rays and X-rays, typically being 5-8 MeV, or 5 to 10 times that of a beta particle and 20 or more times the energy of a gamma ray. Thus, this deposition of a large amount of energy over a very short distance gives α-radiation an exceptionally high linear energy transfer (LET), high relative biological efficacy (RBE) and low oxygen enhancement ratio (OER) compared to gamma and beta radiation (see Hall, “Radiobiology for the radiologist”, Fifth edition, Lippincott Williams & Wilkins, Philadelphia PA, USA, 2000). This explains the exceptional cytotoxicity of alpha emitting radionuclides and also imposes stringent demands on the biological targeting of such isotopes and upon the level of control and study of alpha emitting radionuclide distribution which is necessary in order to avoid unacceptable side effects.

Several alpha-emitters, such as Terbium-149 (¹⁴⁹Tb), Astatine-211 (²¹¹At), Bismuth-212 (²¹²Bi), Bismuth-213 (²¹³Bi), Actinium-225 (²²⁵Ac), Radium-223 (²²³Ra), Radium-224 (²²⁴Ra), or Thorium-227 (²²⁷Th), have been investigated and/or commercialised for use as radiopharmaceuticals. In particular, the use of ‘tissue-targeting’ radiopharmaceuticals has meant that the radioactive nucleus can be delivered to the target cell (for example a cancerous cell) with an improved accuracy, thus minimising unwanted damage to surrounding tissue and hence minimising side effects. Tissue-targeting radiopharmaceuticals are typically conjugates in which the radiopharmaceutical moiety is linked to a targeting unit, for example via a chelator. The targeting unit (for example, an antibody) guides the radiopharmaceutical to the desired cell (by targeting a particular antigen on a cancer cell for example) such that the alpha radiation can be delivered in close proximity to the target. A small number of elements can be considered “self targeting” due to their inherent properties. Radium, for example, is a calcium analogue and targets bone surfaces by this inherent nature however its utility is limited by the paucity of chelating agents which effectively complex radium with high enough stability to be useful in vivo when conjugated to targeted ligands. Henriksen et al. [Applied Radiation and Isotopes 56, 2002, 667] reported on the kinetic and thermodynamic properties of chelating agents DOTA, DTPA, kryptofix 2.2.2 and calix[4]-tetraacetic acid the latter possessing the best properties. However the rapid dissociation of the complex indicated that these monomeric chelator systems would not be useful in vivo due to poor stability.

More recently Thiele et al. reported on the macropa chelator having the highest affinity for Ba²⁺ at pH 7.4 [J Am Chem Soc 2018, 140(49)17071]. This ligand appeared also to possess excellent selectivity for large over small alkaline earth metals. The same authors have subsequently presented (EANM, 2019) work demonstrating that this chelator at high concentration does indeed form a complex with radium-223 at chelator concentrations in the millimolar range. Unfortunately all attempts to label conjugates comprising macropa covalently linked to a targeting ligand at the concentrations useful for targeted alpha therapy failed due to the poor instability of complexes of the mono-chelator-conjugate derivatives.

However, the state of the art does not describe multimers of macropa having sufficient stability to be useful in targeted alpha therapy. It has now been found, and this constitutes the basis of the present invention, that the compounds of the present invention have surprising and advantageous properties.

In particular, the compounds of the present invention have sufficient stability to be useful in targeted alpha therapy as multiple chelator interactions between donor atoms contribute to complex stabilisation in the concentration range enabling targeted alpha therapy.

Interestingly, multimers possess beneficial properties in terms of tailoring the pharmacodynamic and pharmacokinetic properties of targeted conjugates of this invention. In particular conjugates were found to have reduced bone uptake resulting in reduced myelosuppression in rodent models leading to a surprising improvement in survival.

### DESCRIPTION OF THE INVENTION

In accordance with a first aspect, the present invention covers compounds of general formula (I):

[(C)n-L]-(V)m (I)

where C is a chelator and >1, L is a multi-functional linker moiety comprising multiple functional groups for the covalent attachment of chelator such as a polyamine or polyacid-containing backbone or amino acid containing polymer comprising side-chains with amino, thiol or carboxylic acid moieties such as lysine, cysteine or glutamic acid and V is a tissue targeting moiety where m=1-5 which preferentially coupled through a coupling moiety to either the multifunctional linker moiety L or directly to the chelator moiety C, and stereoisomers, tautomers, N-oxides, hydrates, solvates, and salts thereof, and mixtures of same.

Preferred n's of general formula (I) are 2, 4, 8, 16 and 32

Chelators capable of complexing a metal, said metal being a radioactive isotope defined herein, are known. Non-limiting examples of chelators can be found in Q J Nucl Med Mol Imaging, 2008 June; 52(2); 166-173.

In a first embodiment of the first aspect, C is the macrocyclic chelating agent macropa-NH₂ below:

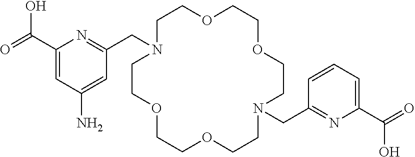 [SMILES: Nc1cc(CN2CCOCCOCCN(Cc3cccc(C(=O)O)n3)CCOCCOCC2)nc(C(=O)O)c1]

wherein either the amino substituent group or the carboxylic acid groups are used to form amide bonds with either L or V.

In one embodiment, the compound (Tet1) comprises 4 macropa units linked via a tetraamino backbone modified with the diglycolic acid spacer:

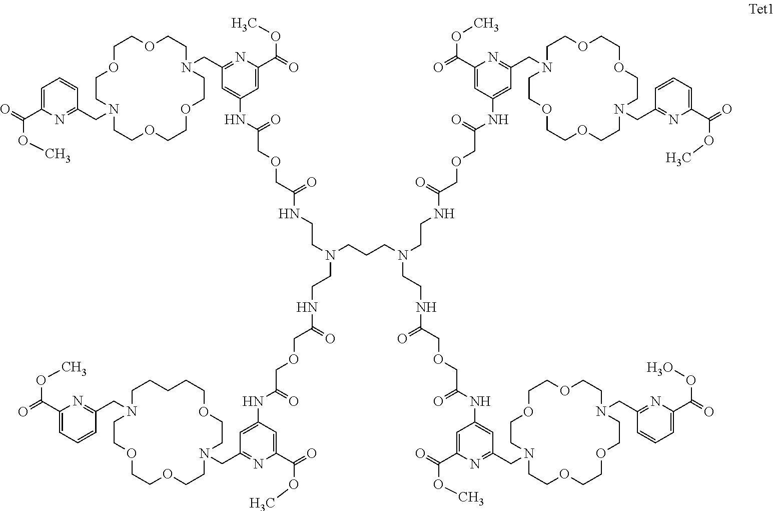 [SMILES: COC(=O)c1cccc(CN2CCCCCOCCN(Cc3cc(NC(=O)COCC(=O)NCCN(CCCN(CCNC(=O)COCC(=O)Nc4cc(CN5CCOCCOCCN(Cc6cccc(C(=O)OC)n6)CCOCCOCC5)nc(C(=O)OC)c4)CCNC(=O)COCC(=O)Nc4cc(CN5CCOCCOCCN(Cc6cccc(C(=O)OO)n6)CCOCCOCC5)nc(C(=O)OC)c4)CCNC(=O)COCC(=O)Nc4cc(CN5CCOCCOCCN(Cc6cccc(C(=O)OC)n6)CCOCCOCC5)nc(C(=O)OC)c4)cc(C(=O)OC)n3)CCOCCOCC2)n1]

In another embodiment the ester functions of compound Tet1 are hydrolysed to yield Compound Tet2. This tetra-macropa compound bears 8 carboxylic acid groups which can be utilised for the further conjugation of the chelating agent to a targeting moiety through amide bond formation. In a preferred embodiment this targeting agent is a monoclonal antibody.

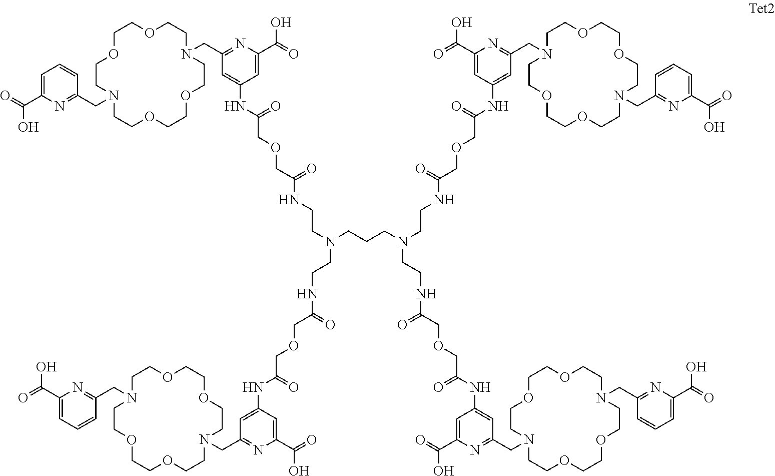 [SMILES: O=C(COCC(=O)Nc1cc(CN2CCOCCOCCN(Cc3cccc(C(=O)O)n3)CCOCCOCC2)nc(C(=O)O)c1)NCCN(CCCN(CCNC(=O)COCC(=O)Nc1cc(CN2CCOCCOCCN(Cc3cccc(C(=O)O)n3)CCOCCOCC2)nc(C(=O)O)c1)CCNC(=O)COCC(=O)Nc1cc(CN2CCOCCOCCN(Cc3cccc(C(=O)O)n3)CCOCCOCC2)nc(C(=O)O)c1)CCNC(=O)COCC(=O)Nc1cc(CN2CCOCCOCCN(Cc3cccc(C(=O)O)n3)CCOCCOCC2)nc(C(=O)O)c1]

In another embodiment the DOTA chelator is used to make multimeric compounds, such as e.g. tetra-DOTA as depicted below, said compounds can be utilised for the further conjugation to a targeting moiety. It should be obvious to one skilled in the art what constitutes a radiometal suitable for complexation to DOTA chelator, e.g. Y-90, Lu-177, Ac-225, Th-227, Bi-212, Bi-213.

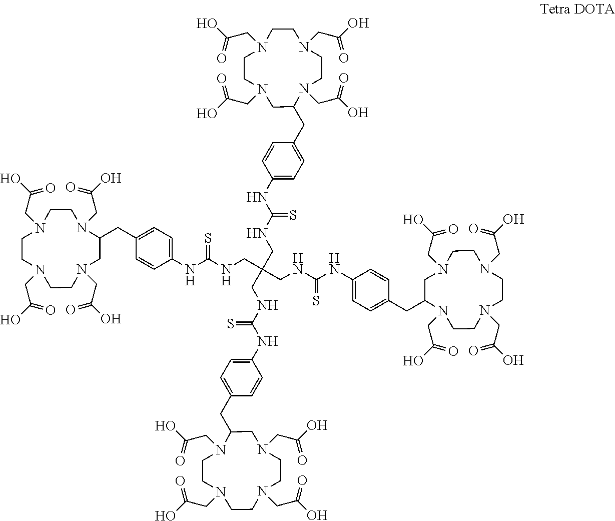 [SMILES: O=C(O)CN1CCN(CC(=O)O)CCN(CC(=O)O)C(Cc2ccc(NC(=S)NCC(CNC(=S)Nc3ccc(CC4CN(CC(=O)O)CCN(CC(=O)O)CCN(CC(=O)O)CCN4CC(=O)O)cc3)(CNC(=S)Nc3ccc(CC4CN(CC(=O)O)CCN(CC(=O)O)CCN(CC(=O)O)CCN4CC(=O)O)cc3)CNC(=S)Nc3ccc(CC4CN(CC(=O)O)CCN(CC(=O)O)CCN(CC(=O)O)CCN4CC(=O)O)cc3)cc2)CN(CC(=O)O)CC1]

In a second embodiment of the first aspect, C is the macrocyclic chelating agent macropa-CH₂CH₂—COOH below:

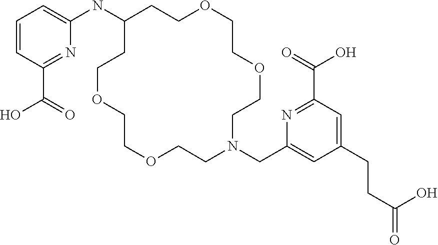 [SMILES: O=C(O)CCc1cc(CN2CCOCCOCCC(Nc3cccc(C(=O)O)n3)CCOCCOCC2)nc(C(=O)O)c1]

wherein the carboxylic acid groups are used to form amide bonds with either L or V.

In a preferred embodiment, the chelator is linked to the multi-amine-back-bone via a carboxy-ethyl-linker, which is attached to pyridine of the chelator. As depicted below for Tet5.

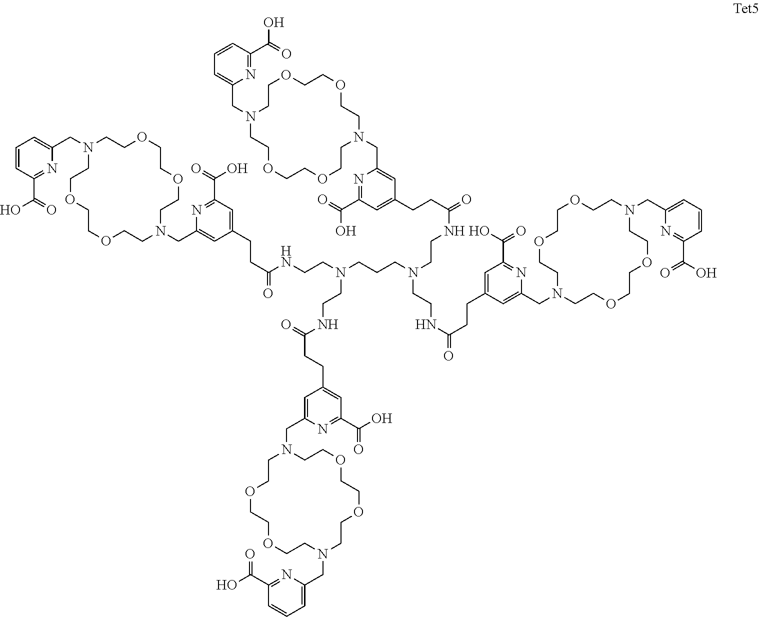 [SMILES: O=C(CCc1cc(CN2CCOCCOCCN(Cc3cccc(C(=O)O)n3)CCOCCOCC2)nc(C(=O)O)c1)NCCN(CCCN(CCNC(=O)CCc1cc(CN2CCOCCOCCN(Cc3cccc(C(=O)O)n3)CCOCCOCC2)nc(C(=O)O)c1)CCNC(=O)CCc1cc(CN2CCOCCOCCN(Cc3cccc(C(=O)O)n3)CCOCCOCC2)nc(C(=O)O)c1)CCNC(=O)CCc1cc(CN2CCOCCOCCN(Cc3cccc(C(=O)O)n3)CCOCCOCC2)nc(C(=O)O)c1]

### DEFINITIONS

As used herein, the term “linker moiety” is used to indicate a chemical entity which serves to join the chelating groups to the core structure, which form a key component in various aspects of the invention. Typically, each chelating moiety (e.g. those of formula I above) will be multidentate and possess a relatively good selectivity for radium isotopes. However only when combined into multimer complexes do we achieve stability acceptable for the use of in vivo targeted radiotherapy. The linker moieties may also serve as the point of attachment between the complexing part and the targeting moiety. In such a case, at least one linker moiety will join to a coupling moiety. Suitable linker moieties include short hydrocarbyl groups, such as C1 to C12 hydrocarbyl, including C1 to C12 alkyl, alkenyl or alkynyl group, including methyl, ethyl, propyl, butyl, pentyl and/or hexyl groups of all topologies.

Linker moieties may also be or comprise any other suitably robust chemical linkages including esters, ethers, amine and/or amide groups. The total number of atoms joining two chelating moieties (counting by the shortest path if more than one path exists) will generally be limited, so as to constrain the chelating moieties in a suitable arrangement for complex formation. Thus, linker moieties will typically be chosen to provide no more than 25 atoms between chelating moieties, preferably, 1 to 15 atoms, and more preferably 5 to 15 atoms between chelating moieties. Where a linker moiety joins two chelating moieties directly, the linker will typically be 1 to 12 atoms in length, preferably 2 to 10 (such as ethyl, propyl, n-butyl etc).

Where the linker moiety joins to a central backbone then each linker may be shorter with two separate linkers joining the chelating moieties. A linker length of 1 to 8 atoms, preferably 1 to 6 atoms may be preferred in this case (methyl, ethyl and propyl being suitable, as are groups such as these having an ester, ether or amide linkage at one end or both).

A “coupling moiety” as used herein serves to join the linker component or chelator to the targeting moiety through stable covalent bond formation such as an amide bond. Preferably, coupling moieties will be present on the chelator allowing direct covalent attachment to the targeting moietyor more typically facilitate attachment through the linker moiety or the backbone. Should two or more coupling moieties be used, each can be attached to any of the available sites such as on any backbone, linker or chelating group.

In one embodiment, the coupling moiety may have the structure:

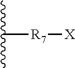

wherein R₇ is a bridging moiety, which is a member selected from substituted or unsubstituted alkyl, substituted or unsubstituted heteroalkyl, substituted or unsubstituted heterocycloalkyl, substituted or unsubstituted aryl and substituted or unsubstituted heteroaryl; and X is a reactive functional group. The preferred bridging moieties include all those groups indicated herein as suitable linker moieties. Preferred targeting moieties include all of those described herein and preferred reactive X groups include any group capable of forming a covalent linkage to a targeting moiety, including, for example, COOH, OH, SH, NHR and COH groups, where the R of NHR may be H or any of the short hydrocarbyl groups described herein. Highly preferred groups for attachment onto the targeting moiety include epsilon-amines of lysine residues and thiol groups of cysteine residues. Non-limiting examples of suitable reactive X groups, include N-hydroxysuccimidylesters, imidoesters, acylhalides, N-maleimides, alpha-halo acetyl and isothiocyanates, where the latter three are suitable for reaction with a thiol group. Conjugation of the chelator-linker component of the invention to the targeting moiety via covalent bond formation can be achieved using ‘click chemistry’ as described in Chem. Rev., 2013, 113, 7, 4905-4979.

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon or nitrogen or sulfur atom. Commonly, it is possible for the number of optional substituents, when present, to be 1, 2, 3, 4 or 5, in particular 1, 2 or 3.

As used herein, the term “one or more”, e.g. in the definition of the substituents of the compounds of general formula (I) of the present invention, means “1, 2, 3, 4 or 5, particularly 1, 2, 3 or 4, more particularly 1, 2 or 3, even more particularly 1 or 2”.

When groups in the compounds according to the invention are substituted, it is possible for said groups to be mono-substituted or poly-substituted with substituent(s), unless otherwise specified. Within the scope of the present invention, the meanings of all groups which occur repeatedly are independent from one another. It is possible that groups in the compounds according to the invention are substituted with one, two or three identical or different substituents, particularly with one substituent.

As used herein, an “oxo substituent” represents an oxygen atom, which is bound to a carbon atom or to a sulfur atom via a double bond.

The term “ring substituent” means a substituent attached to an aromatic or nonaromatic ring which replaces an available hydrogen atom on the ring.

The term “comprising” when used in the specification includes “consisting of”.

If within the present text any item is referred to as “as mentioned herein”, it means that it may be mentioned anywhere in the present text.

The terms as mentioned in the present text have the following meanings:

The term “halogen atom” means a fluorine, chlorine, bromine or iodine atom, particularly a fluorine, chlorine or bromine atom.

The term “C₁-C₆-alkyl” means a linear or branched, saturated, monovalent hydrocarbon group having 1, 2, 3, 4, 5 or 6 carbon atoms, e.g. a methyl, ethyl, propyl, isopropyl, butyl, sec-butyl, isobutyl, tert-butyl, pentyl, isopentyl, 2-methylbutyl, 1-methylbutyl, 1-ethylpropyl, 1,2-dimethylpropyl, neo-pentyl, 1,1-dimethylpropyl, hexyl, 1-methylpentyl, 2-methylpentyl, 3-methylpentyl, 4-methylpentyl, 1-ethylbutyl, 2-ethylbutyl, 1,1-dimethylbutyl, 2,2-dimethylbutyl, 3,3-dimethylbutyl, 2,3-dimethylbutyl, 1,2-dimethylbutyl or 1,3-dimethylbutyl group, or an isomer thereof. Particularly, said group has 1, 2, 3 or 4 carbon atoms (“C₁-C₄-alkyl”), e.g. a methyl, ethyl, propyl, isopropyl, butyl, sec-butyl isobutyl, or tert-butyl group, more particularly 1, 2 or 3 carbon atoms (“C₁-C₃-alkyl”), e.g. a methyl, ethyl, n-propyl or isopropyl group.

The term “C₁-C₆-hydroxyalkyl” means a linear or branched, saturated, monovalent hydrocarbon group in which the term “C₁-C₆-alkyl” is defined supra, and in which 1, 2 or 3 hydrogen atoms are replaced with a hydroxy group, e.g. a hydroxymethyl, 1-hydroxyethyl, 2-hydroxyethyl, 1,2-dihydroxyethyl, 3-hydroxypropyl, 2-hydroxypropyl, 1-hydroxypropyl, 1-hydroxypropan-2-yl, 2-hydroxypropan-2-yl, 2,3-dihydroxypropyl, 1,3-dihydroxypropan-2-yl, 3-hydroxy-2-methyl-propyl, 2-hydroxy-2-methyl-propyl, 1-hydroxy-2-methyl-propyl group.

The term “C₁-C₆-alkylsulfanyl” means a linear or branched, saturated, monovalent group of formula (C₁-C₆-alkyl)—S—, in which the term “C₁-C₆-alkyl” is as defined supra, e.g. a methylsulfanyl, ethylsulfanyl, propylsulfanyl, isopropylsulfanyl, butylsulfanyl, sec-butylsulfanyl, isobutylsulfanyl, tert-butylsulfanyl, pentylsulfanyl, isopentylsulfanyl, hexylsulfanyl group.

The term “C₁-C₆-haloalkyl” means a linear or branched, saturated, monovalent hydrocarbon group in which the term “C₁-C₆-alkyl” is as defined supra, and in which one or more of the hydrogen atoms are replaced, identically or differently, with a halogen atom. Particularly, said halogen atom is a fluorine atom. Said C₁-C₆-haloalkyl group is, for example, fluoromethyl, difluoromethyl, trifluoromethyl, 2-fluoroethyl, 2,2-difluoroethyl, 2,2,2-trifluoroethyl, pentafluoroethyl, 3,3,3-trifluoropropyl or 1,3-difluoropropan-2-yl.

The term “C₁-C₆-alkoxy” means a linear or branched, saturated, monovalent group of formula (C₁-C₆-alkyl)—O—, in which the term “C₁-C₆-alkyl” is as defined supra, e.g. a methoxy, ethoxy, n-propoxy, isopropoxy, n-butoxy, sec-butoxy, isobutoxy, tert-butoxy, pentyloxy, isopentyloxy or n-hexyloxy group, or an isomer thereof.

The term “C₁-C₆-haloalkoxy” means a linear or branched, saturated, monovalent C₁-C₆-alkoxy group, as defined supra, in which one or more of the hydrogen atoms is replaced, identically or differently, with a halogen atom. Particularly, said halogen atom is a fluorine atom. Said C₁-C₆-haloalkoxy group is, for example, fluoromethoxy, difluoromethoxy, trifluoromethoxy, 2,2,2-trifluoroethoxy or pentafluoroethoxy.

The term “C₂-C₆-alkenyl” means a linear or branched, monovalent hydrocarbon group, which contains one or two double bonds, and which has 2, 3, 4, 5 or 6 carbon atoms, particularly 2 or 3 carbon atoms (“C₂-C₃-alkenyl”), it being understood that in the case in which said alkenyl group contains more than one double bond, then it is possible for said double bonds to be isolated from, or conjugated with, each other. Said alkenyl group is, for example, an ethenyl (or “vinyl”), prop-2-en-1-yl (or “allyl”), prop-1-en-1-yl, but-3-enyl, but-2-enyl, but-1-enyl, pent-4-enyl, pent-3-enyl, pent-2-enyl, pent-1-enyl, hex-5-enyl, hex-4-enyl, hex-3-enyl, hex-2-enyl, hex-1-enyl, prop-1-en-2-yl (or “isopropenyl”), 2-methylprop-2-enyl, 1-methylprop-2-enyl, 2-methylprop-1-enyl, 1-methylprop-1-enyl, 3-methylbut-3-enyl, 2-methylbut-3-enyl, 1-methylbut-3-enyl, 3-methylbut-2-enyl, 2-methylbut-2-enyl, 1-methylbut-2-enyl, 3-methylbut-1-enyl, 2-methylbut-1-enyl, 1-methylbut-1-enyl, 1, 1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, 3-methylpent-3-enyl, 2-methylpent-3-enyl, 1-methylpent-3-enyl, 4-methylpent-2-enyl, 3-methylpent-2-enyl, 2-methylpent-2-enyl, 1-methylpent-2-enyl, 4-methylpent-1-enyl, 3-methylpent-1-enyl, 2-methylpent-1-enyl, 1-methylpent-1-enyl, 3-ethylbut-3-enyl, 2-ethylbut-3-enyl, 1-ethylbut-3-enyl, 3-ethylbut-2-enyl, 2-ethylbut-2-enyl, 1-ethylbut-2-enyl, 3-ethylbut-1-enyl, 2-ethylbut-1-enyl, 1-ethylbut-1-enyl, 2-propylprop-2-enyl, 1-propylprop-2-enyl, 2-isopropylprop-2-enyl, 1-isopropylprop-2-enyl, 2-propylprop-1-enyl, 1-propylprop-1-enyl, 2-isopropylprop-1-enyl, 1-isopropylprop-1-enyl, 3,3-dimethylprop-1-enyl, 1-(1,1-dimethylethyl)ethenyl, buta-1,3-dienyl, penta-1,4-dienyl or hexa-1,5-dienyl group. Particularly, said group is vinyl or allyl.

The term “C₂-C₆-alkynyl” means a linear or branched, monovalent hydrocarbon group which contains one triple bond, and which contains 2, 3, 4, 5 or 6 carbon atoms, particularly 2 or 3 carbon atoms (“C₂-C₃-alkynyl”). Said C₂-C₆-alkynyl group is, for example, ethynyl, prop-1-ynyl, prop-2-ynyl (or “propargyl”), but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methyl-pent-4-ynyl, 2-methylpent-3-ynyl, 1-methylpent-3-ynyl, 4-methylpent-2-ynyl, 1-methyl-pent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2,2-dimethylbut-3-ynyl, 1,1-dimethylbut-3-ynyl, 1,1-dimethylbut-2-ynyl or 3,3-dimethylbut-1-ynyl group.

The term “C₃-C₈-cycloalkyl” means a saturated, monovalent, mono- or bicyclic hydrocarbon ring which contains 3, 4, 5, 6, 7 or 8 carbon atoms (“C₃-C₈-cycloalkyl”). Said C₃-C₈-cycloalkyl group is for example, a monocyclic hydrocarbon ring, e.g. a cyclopropyl, cyclobutyl, cyclopentyl, cyclohexyl, cycloheptyl or cyclooctyl group, or a bicyclic hydrocarbon ring, e.g. a bicyclo[4.2.0]octyl or octahydropentalenyl.

The term “C₄-C₈-cycloalkenyl” means a monovalent, mono- or bicyclic hydrocarbon ring which contains 4, 5, 6, 7 or 8 carbon atoms and one double bond. Particularly, said ring contains 4, 5 or 6 carbon atoms (“C₄-C₆-cycloalkenyl”). Said C₄-C₈-cycloalkenyl group is for example, a monocyclic hydrocarbon ring, e.g. a cyclobutenyl, cyclopentenyl, cyclohexenyl, cycloheptenyl or cyclooctenyl group, or a bicyclic hydrocarbon ring, e.g. a bicyclo[2.2.1]hept-2-enyl or bicyclo[2.2.2]oct-2-enyl.

The term “C₃-C₈-cycloalkoxy” means a saturated, monovalent, mono- or bicyclic group of formula (C₃-C₈-cycloalkyl)—O—, which contains 3, 4, 5, 6, 7 or 8 carbon atoms, in which the term “C₃-C₈-cycloalkyl” is defined supra, e.g. a cyclopropyloxy, cyclobutyloxy, cyclopentyloxy, cyclohexyloxy, cycloheptyloxy or cyclooctyloxy group.

The term “spirocycloalkyl” means a saturated, monovalent bicyclic hydrocarbon group in which the two rings share one common ring carbon atom, and wherein said bicyclic hydrocarbon group contains 5, 6, 7, 8, 9, 10 or 11 carbon atoms, it being possible for said spirocycloalkyl group to be attached to the rest of the molecule via any one of the carbon atoms except the spiro carbon atom. Said spirocycloalkyl group is, for example, spiro[2.2]pentyl, spiro[2.3]hexyl, spiro[2.4]heptyl, spiro[2.5]octyl, spiro[2.6]nonyl, spiro[3.3]heptyl, spiro[3.4]octyl, spiro[3.5]nonyl, spiro[3.6]decyl, spiro[4.4]nonyl, spiro[4.5]decyl, spiro[4.6]undecyl or spiro[5.5]undecyl.

The terms “4- to 7-membered heterocycloalkyl” and “4- to 6-membered heterocycloalkyl” meal a monocyclic, saturated heterocyde with 4, 5, 6 or 7 or, respectively, 4, 5 or 6 ring atoms in total, which contains one or two identical or different ring heteroatoms from the series N, O and S, it being possible for said heterocycloalkyl group to be attached to the rest of the molecule via any one of the carbon atoms or, if present, a nitrogen atom.

Said heterocycloalkyl group, without being limited thereto, can be a 4-membered ring, such as azetidinyl, oxetanyl or thietanyl, for example; ora 5-membered ring, such as tetrahydrofuranyl, 1,3-dioxolanyl, thiolanyl, pyrrolidinyl, imidazolidinyl, pyrazolidinyl, 1,1-dioxidothiolanyl, 1,2-oxazolidinyl, 1,3-oxazolidinyl or 1,3-thiazolidinyl, for example; or a 6-membered ring, such as tetrahydropyranyl, tetrahydrothiopyranyl, piperidinyl, morpholinyl, dithianyl, thiomorpholinyl, piperazinyl, 1,3-dioxanyl, 1,4-dioxanyl or 1,2-oxazinanyl, for example, or a 7-membered ring, such as azepanyl, 1,4-diazepanyl or 1,4-oxazepanyl, for example.

Particularly, “4- to 6-membered heterocycloalkyl” means a 4- to 6-membered heterocycloalkyl as defined supra containing one ring nitrogen atom and optionally one further ring heteroatom from the series: N, O, S. More particularly, “5- or 6-membered heterocycloalkyl” means a monocyclic, saturated heterocycle with 5 or 6 ring atoms in total, containing one ring nitrogen atom and optionally one further ring heteroatom from the series: N, O.

The term “5- to 8-membered heterocycloalkenyl” means a monocyclic, unsaturated, non-aromatic heterocycle with 5, 6, 7 or 8 ring atoms in total, which contains one or two double bonds and one or two identical or different ring heteroatoms from the series: N, O, S; it being possible for said heterocycloalkenyl group to be attached to the rest of the molecule via any one of the carbon atoms or, if present, a nitrogen atom.

Said heterocycloalkenyl group is, for example, 4H-pyranyl, 2H-pyranyl, 2,5-dihydro-1H-pyrrolyl, [1,3]olioxolyl, 4H-[1,3,4]thiadiazinyl, 2,5-dihydrofuranyl, 2,3-dihydrofuranyl, 2,5-dihydrothio-phenyl, 2,3-dihydrothiophenyl, 4,5-dihydrooxazolyl or 4H-[1,4]thiazinyl.

The term “heterospirocycloalkyl” means a bicyclic, saturated heterocycle with 6, 7, 8, 9, 10 or 11 ring atoms in total, in which the two rings share one common ring carbon atom, which “heterospirocycloalkyl” contains one or two identical or different ring heteroatoms from the series: N, O, S; it being possible for said heterospirocycloalkyl group to be attached to the rest of the molecule via any one of the carbon atoms, except the spiro carbon atom, or, if present, a nitrogen atom.

Said heterospirocycloalkyl group is, for example, azaspiro[2.3]hexyl, azaspiro[3.3]heptyl, oxaazaspiro[3.3]heptyl, thiaazaspiro[3.3]heptyl, oxaspiro[3.3]heptyl, oxazaspiro[5.3]nonyl, oxazaspiro[4.3]octyl, azaspiro[4,5]decyl, oxazaspiro [5.5]undecyl, diazaspiro[3.3]heptyl, thiazaspiro[3.3]heptyl, thiazaspiro[4.3]octyl, azaspiro[5.5]undecyl, or one of the further homologous scaffolds such as spiro[3.4]-, spiro[4.4]-, spiro[2.4]-, spiro[2.5]-, spiro[2.6]-, spiro[3.5]-, spiro[3.6]-, spiro[4.5]- and spiro[4.6]-.

The term “fused heterocydoalkyl” means a bicyclic, saturated heterocycle with 6, 7, 8, 9 or 10 ring atoms in total, in which the two rings share two adjacent ring atoms, which “fused heterocycloalkyl” contains one or two identical or different ring heteroatoms from the series: N, O, S; it being possible for said fused heterocycloalkyl group to be attached to the rest of the molecule via any one of the carbon atoms or, if present, a nitrogen atom.

Said fused heterocycloalkyl group is, for example, azabicyclo[3.3.0]octyl, azabicyclo[4.3.0]nonyl, diazabicyclo[4.3.0]nonyl, oxazabicyclo[4.3.0]nonyl, thiazabicyclo[4.3.0]nonyl or azabicyclo[4.4.0]decyl.

The term “bridged heterocydoalkyl” means a bicyclic, saturated heterocycle with 7, 8, 9 or 10 ring atoms in total, in which the two rings share two common ring atoms which are not adjacent, which “bridged heterocycloalkyl” contains one or two identical or different ring heteroatoms from the series: N, O, S; it being possible for said bridged heterocycloalkyl group to be attached to the rest of the molecule via any one of the carbon atoms, except the spiro carbon atom, or, if present, a nitrogen atom.

Said bridged heterocycloalkyl group is, for example, azabicyclo[2.2.1]heptyl, oxazabicyclo[2.2.1]heptyl, thiazabicyclo[2.2.1]heptyl, diazabicyclo[2.2.1]heptyl, azabicyclo-[2.2.2]octyl, diazabicyclo[2.2.2]octyl, oxazabicyclo[2.2.2]octyl, thiazabicyclo[2.2.2]octyl, azabi-cyclo[3.2.1]octyl, diazabicyclo[3.2.1]octyl, oxazabicyclo[3.2.1]octyl, thiazabicyclo[3.2.1]octyl, azabicyclo[3.3.1]nonyl, diazabicyclo[3.3.1]nonyl, oxazabicyclo[3.3.1]nonyl, thiazabicyclo[3.3.1]-nonyl, azabicyclo[4.2.1]nonyl, diazabicyclo[4.2.1]nonyl, oxazabicyclo[4.2.1]nonyl, thiaza-bicyclo[4.2.1]nonyl, azabicyclo[3.3.2]decyl, diazabicyclo[3.3.2]decyl, oxazabicyclo[3.3.2]decyl, thiazabicyclo[3.3.2]decyl or azabicyclo[4.2.2]decyl.

The term “heteroaryl” means a monovalent, monocyclic, bicyclic or tricyclic aromatic ring having 5, 6, 8, 9, 10, 11, 12, 13 or 14 ring atoms (a “5- to 14-membered heteroaryl” group), particularly 5, 6, 9 or 10 ring atoms, which contains at least one ring heteroatom and optionally one, two or three further ring heteroatoms from the series: N, O and/or S, and which is bound via a ring carbon atom or optionally via a ring nitrogen atom (if allowed by valency).

Said heteroaryl group can be a 5-membered heteroaryl group, such as, for example, thienyl, furanyl, pyrrolyl, oxazolyl, thiazolyl, imidazolyl, pyrazolyl, isoxazolyl, isothiazolyl, oxadiazolyl, triazolyl, thiadiazolyl or tetrazolyl; or a 6-membered heteroaryl group, such as, for example, pyridinyl, pyridazinyl, pyrimidinyl, pyrazinyl or triazinyl; or a tricyclic heteroaryl group, such as, for example, carbazolyl, acridinyl or phenazinyl; or a 9-membered heteroaryl group, such as, for example, benzofuranyl, benzothienyl, benzoxazolyl, benzisoxazolyl, benzimidazolyl, benzothiazolyl, benzotriazolyl, indazolyl, indolyl, isoindolyl, indolizinyl or purinyl; or a 10-membered heteroaryl group, such as, for example, quinolinyl, quinazolinyl, isoquinolinyl, cinnolinyl, phthalazinyl, quinoxalinyl or pteridinyl.

In general, and unless otherwise mentioned, the heteroaryl or heteroarylene groups include all possible isomeric forms thereof, e.g.: tautomers and positional isomers with respect to the point of linkage to the rest of the molecule. Thus, for some illustrative non-restricting examples, the term pyridinyl includes pyridin-2-yl, pyridin-3-yl and pyridin-4-yl; or the term thienyl includes thien-2-yl and thien-3-yl.

The term “C₁-C₆”, as used in the present text, e.g. in the context of the definition of “C₁-C₆-alkyl”, “C₁-C₆-haloalkyl”, “C₁-C₆-hydroxyalkyl”, “C₁-C₆-alkoxy” or “C₁-C₆-haloalkoxy” means an alkyl group having a finite number of carbon atoms of 1 to 6, i.e. 1, 2, 3, 4, 5 or 6 carbon atoms.

Further, as used herein, the term “C₃-C₈”, as used in the present text, e.g. in the context of the definition of “C₃-C₈-cycloalkyl”, means a cycloalkyl group having a finite number of carbon atoms of 3 to 8, i.e. 3, 4, 5, 6, 7 or 8 carbon atoms.

When a range of values is given, said range encompasses each value and sub-range within said range.

For example:
“C₁-C₆” encompasses C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆;
“C₂-C₆” encompasses C₂, C₃, C₄, C₅, C₆, C₂-C₆, C₂-C₅, C₂-C₄, C₂-C₃, C₃-C₆, C₃-C₅, C₃-C₄, C₄-C₆, C₄-C₅, and C₅-C₆;
“C₃-C₁₀” encompasses C₃, C₄, C₅, C₆, C₇, C₈, C₉, C₁₀, C₃-C₁₀, C₃-C₉, C₃-C₈, C₃-C₇, C₃-C₆, C₃-C₅, C₃-C₄, C₄-C₁₀, C₄-C₉, C₄-C₈, C₄-C₇, C₄-C₆, C₄-C₅, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉-C₁₀;
“C₃-C₈” encompasses C₃, C₄, C₅, C₆, C₇, C₈, C₃-C₈, C₃-C₇, C₃-C₆, C₃-C₅, C₃-C₄, C₄-C₈, C₄-C₇, C₄-C₆, C₄-C₅, C₅-C₈, C₅-C₇, C₅-C₆, C₆-C₈, C₆-C₇ and C₇-C₈;
“C₃-C₆” encompasses C₃, C₄, C₅, C₆, C₃-C₆, C₃-C₅, C₃-C₄, C₄-C₆, C₄-C₅, and C₅-C₆;
“C₄-C₈” encompasses C₄, C₅, C₆, C₇, C₈, C₄-C₈, C₄-C₇, C₄-C₆, C₄-C₅, C₅-C₈, C₅-C₇, C₅-C₆, C₆-C₈, C₆-C₇ and C₇-C₈;
“C₄-C₇” encompasses C₄, C₅, C₆, C₇, C₄-C₇, C₄-C₆, C₄-C₅, C₅-C₇, C₅-C₆ and C₆-C₇;
“C₄-C₆” encompasses C₄, C₅, C₆, C₄-C₆, C₄-C₅ and C₅-C₆;
“C₅-C₁₀” encompasses C₅, C₆, C₇, C₈, C₉, C₁₀, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉-C₁₀;
“C₆-C₁₀” encompasses C₆, C₇, C₈, C₉, C₁₀, C₆-C₁₀, C₆-C₉, C₆-C₈, C₆-C₇, C₇-C₁₀, C₇-C₉, C₇-C₈, C₈-C₁₀, C₈-C₉ and C₉-C₁₀.

As used herein, the term “leaving group” means an atom or a group of atoms that is displaced in a chemical reaction as stable species taking with it the bonding electrons. In particular, such a leaving group is selected from the group comprising: halide, in particular fluoride, chloride, bromide or iodide, (methylsulfonyl)oxy, [(trifluoromethyl)sulfonyl]oxy, [(nonafluorobutyl)-sulfonyl]oxy, (phenylsulfonyl)oxy, [(4-methylphenyl)sulfonyl]oxy, [(4-bromophenyl)sulfonyl]oxy, [(4-nitrophenyl)sulfonyl]oxy, [(2-nitrophenyl)suifonyl]oxy, [(4-isopropylphenyl)sulfonyl]oxy, [(2,4,6-triisopropylphenyl)sulfonyl]oxy, [(2,4,6-trimethylphenyl)sulfonyl]oxy, [(4-tert-butyl-phenyl)sulfonyl]oxy and [(4-methoxyphenyl)sulfonyl]oxy.

It is possible forthe compounds of general formula (I) to exist as isotopic variants. The invention therefore includes one or more isotopic variant(s) of the compounds of general formula (I), particularly deuterium-containing compounds of general formula (I).

The term “Isotopic variant” of a compound or a reagent is defined as a compound exhibiting an unnatural proportion of one or more of the isotopes that constitute such a compound.

The term “Isotopic variant of the compound of general formula (I)” is defined as a compound of general formula (I) exhibiting an unnatural proportion of one or more of the isotopes that constitute such a compound.

The expression “unnatural proportion” means a proportion of such isotope which is higher than its natural abundance. The natural abundances of isotopes to be applied in this context are described in “Isotopic Compositions of the Elements 1997”, Pure Appl. Chem., 70(1), 217-235, 1998.

Examples of such isotopes include stable and radioactive isotopes of hydrogen, carbon, nitrogen, oxygen, phosphorus, sulfur, fluorine, chlorine, bromine and iodine, such as ²H (deuterium), ³H (tritium), ¹¹C, ¹³C, ¹⁴C, ¹⁵N , ¹⁷O, ¹⁸O, ³²P, ³³P, ³³S, ³⁴S, ³⁵S , ³⁶S , ¹⁸F , ³⁶Cl, ⁸²Br, ¹²³I, ¹²⁴I, ¹²⁵I, ¹²⁹I and ¹³¹I, respectively.

With respect to the treatment and/or prophylaxis of the disorders specified herein the isotopic variant(s) of the compounds of general formula (I) preferably contain deuterium (“deuterium-containing compounds of general formula (I)”). Isotopic variants of the compounds of genera formula (I) in which one or more radioactive isotopes, such as ³H or ¹⁴C, are incorporated are useful e.g. in drug and/or substrate tissue distribution studies. These isotopes are particularly preferred for the ease of their incorporation and detectability. Positron emitting isotopes such as ¹⁸F or ¹¹C may be incorporated into a compound of general formula (I). These isotopic variants of the compounds of general formula (I) are useful for in vivo imaging applications. Deuterium-containing and ¹³C-containing compounds of general formula (I) can be used in mass spectrometry analyses in the context of preclinical or clinical studies.

Isotopic variants of the compounds of general formula (I) can generally be prepared by methods known to a person skilled in the art, such as those described in the schemes and/or examples herein, by substituting a reagent for an isotopic variant of said reagent, preferably for a deuterium-containing reagent. Depending on the desired sites of deuteration, in some cases deuterium from D₂O can be incorporated either directly into the compounds or into reagents that are useful for synthesizing such compounds. Deuterium gas is also a useful reagent for incorporating deuterium into molecules. Catalytic deuteration of olefinic bonds and acetylenic bonds is a rapid route for incorporation of deuterium. Metal catalysts (i.e. Pd, Pt, and Rh) in the presence of deuterium gas can be used to directly exchange deuteriumforhydrogen in functional groups containing hydrocarbons. A variety of deuterated reagents and synthetic building blocks are commercially available from companies such as for example C/D/N Isotopes, Quebec, Canada; Cambridge Isotope Laboratories Inc., Andover, MA, USA; and CombiPhos Catalysts, Inc., Princeton, NJ, USA.

The term “deuterium-containing compound of general formula (I)” is defined as a compound of general formula (I), in which one or more hydrogen atom(s) is/are replaced by one or more deuterium atom(s) and in which the abundance of deuterium at each deuterated position of the compound of general formula (I) is higher than the natural abundance of deuterium, which is about 0.015%. Particularly, in a deuterium-containing compound of general formula (I) the abundance of deuterium at each deuterated position of the compound of general formula (I) is higher than 10%, 20%, 30%, 40%, 50%, 60%, 70% or 80%, preferably higher than 90%, 95%, 96% or 97%, even more preferably higher than 98% or 99% at said position(s). It is understood that the abundance of deuterium at each deuterated position is independent of the abundance of deuterium at other deuterated position(s).

The selective incorporation of one or more deuterium atom(s) into a compound of general formula (I) may alter the physicochemical properties (such as for example acidity [C. L. Perrin, et al., J. Am. Chem. Soc., 2007, 129, 4490], basicity [C. L. Perrin et al., J. Am. Chem. Soc., 2005, 127, 9641], lipophilicity [B. Testa et al., Int. J. Pharm., 1984, 19(3), 271]) and/or the metabolic profile of the molecule and may result in changes in the ratio of parent compound to metabolites or in the amounts of metabolites formed. Such changes may result in certain therapeutic advantages and hence may be preferred in some circumstances. Reduced rates of metabolism and metabolic switching, where the ratio of metabolites is changed, have been reported (A. E. Mutlib et al., Toxicol. Appl. Pharmacol., 2000, 169, 102). These changes in the exposure to parent drug and metabolites can have important consequences with respect to the pharmacodynamics, tolerability and efficacy of a deuterium-containing compound of genera formula (I). In some cases deuterium substitution reduces or eliminates the formation of an undesired or toxic metabolite and enhances the formation of a desired metabolite (e.g. Nevirapine: A. M. Sharma et al., Chem. Res. Toxicol., 2013, 26, 410; Efavirenz: A. E. Mutlib et al., Toxicol. Appl. Pharmacol., 2000, 169, 102). In other cases the major effect of deuteration is to reduce the rate of systemic clearance. As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. ML-337 (C. J. Wenthur et al., J. Med. Chem., 2013, 56, 5208) and Odanacatib (K. Kassahun et al., WO2012/112363) are examples for this deuterium effect. Still other cases have been reported in which reduced rates of metabolism result in an increase in exposure of the drug without changing the rate of systemic clearance (e.g. Rofecoxib: F. Schneider et al., Arzneim. Forsch./Drug. Res., 2006, 56, 295; Telaprevir: F. Maltais et al., J. Med. Chem., 2009, 52, 7993). Deuterated drugs showing this effect may have reduced dosing requirements (e.g. lower number of doses or lower dosage to achieve the desired effect) and/or may produce lower metabolite loads.

A compound of general formula (I) may have multiple potential sites of attack for metabolism. To optimize the above-described effects on physicochemical properties and metabolic profile, deuterium-containing compounds of general formula (I) having a certain pattern of one or more deuterium-hydrogen exchange(s) can be selected. Particularly, the deuterium atom(s) of deuterium-containing compound(s) of general formula (I) is/are attached to a carbon atom and/or is/are located at those positions of the compound of general formula (I), which are sites of attack for metabolizing enzymes such as e.g. cytochrome P₄₅₀.

Where the plural form of the word compounds, salts, polymorphs, hydrates, solvates and the like, is used herein, this is taken to mean also a single compound, salt, polymorph, isomer, hydrate, solvate or the like.

By “stable compound’ or “stable structure” is meant a compound that is sufficiently robust to survive isolation to a useful degree of purity from a reaction mixture, and formulation into an efficacious therapeutic agent.

The compounds of the present invention optionally contain one or more asymmetric centres, depending upon the location and nature of the various substituents desired. It is possible that one or more asymmetric carbon atoms are present in the (R) or (S) configuration, which can result in racemic mixtures in the case of a single asymmetric centre, and in diastereomeric mixtures in the case of multiple asymmetric centres. In certain instances, it is possible that asymmetry also be present due to restricted rotation about a given bond, for example, the central bond adjoining two substituted aromatic rings of the specified compounds.

Preferred compounds are those which producethe more desirable biological activity. Separated, pure or partially purified isomers and stereoisomers or racemic or diastereomeric mixtures of the compounds of the present invention are also included within the scope of the present invention. The purification and the separation of such materials can be accomplished by standard techniques known in the art.

Preferred isomers are those which produce the more desirable biological activity. These separated, pure or partially purified isomers or racemic mixtures of the compounds of this invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., HPLC columns using a chiral phase), with or without conventional derivatisation, optimally chosen to maximise the separation of the enantiomers. Suitable HPLC columns using a chiral phase are commercially available, such as those manufactured by Daicel, e.g., Chiracel OD and Chiracel OJ, for example, among many others, which are all routinely selectable. Enzymatic separations, with or without derivatisation, are also useful. The optically active compounds of the present invention can likewise be obtained by chiral syntheses utilizing optically active starting materials.

In order to distinguish different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. (R)- or (S)-isomers, in any ratio. Isolation of a single stereoisomer, e.g. a single enantiomer or a single diastereomer, of a compound of the present invention is achieved by any suitable state of the art method, such as chromatography, especially chiral chromatography, for example.

Further, it is possible for the compounds of the present invention to exist as tautomers. For example, any compound of the present invention which contains an imidazopyridine moiety as a heteroaryl group for example can exist as a 1H tautomer, or a 3H tautomer, or even a mixture in any amount of the two tautomers, namely:

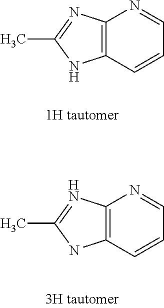

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also covers useful forms of the compounds of the present invention, such as metabolites, hydrates, solvates, prodrugs, salts, in particular pharmaceutically acceptable salts, and/or co-precipitates.

The compounds of the present invention can exist as a hydrate, or as a solvate, wherein the compounds of the present invention contain polar solvents, in particular water, methanol or ethanol for example, as structural element of the crystal lattice of the compounds. It is possible for the amount of polar solvents, in particular water, to exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, it is possible for the compounds of the present invention to exist in free form, e.g. as a free base, or as a free acid, or as a zwitterion, or to exist in the form of a salt. Said salt may be any salt, either an organicor inorganicaddition salt, particularly any pharmaceutically acceptable organic or inorganic addition salt, which is customarily used in pharmacy, or which is used, for example, for isolating or purifying the compounds of the present invention.

The term “pharmaceutically acceptable salt” refers to an inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, or “mineral acid”, such as hydrochloric, hydrobromic, hydroiodic, sulfuric, sulfamic, bisulf uric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, 3-phenylpropionic, pivalic, 2-hydroxyethanesulfonic, itaconic, trifluoromethanesulfonic, dodecylsulf uric, ethanesulfonic, benzenesulfonic, para-toluenesulfonic, methanesulfonic, 2-naphthalenesulfonic, naphthalinedisutionic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium, magnesium or strontium salt, or an aluminium ora zinc salt, or an ammonium salt derived from ammonia or from an organic primary, secondary or tertiary amine having 1 to 20 carbon atoms, such as ethylamine, diethylamine, triethylamine, ethyldiisopropylamine, monoethanolamine, diethanolamine, triethanolamine, dicyclohexylamine, dimethylaminoethanol, diethylaminoethanol, tris(hydroxymethyl)aminomethane, procaine, dibenzylamine, N-methylmorpholine, arginine, lysine, 1,2-ethylenediamine, N-methylpiperidine, N-methyl-glucamine, N,N-dimethyl-glucamine, N-ethyl-glucamine, 1,6-hexanediamine, glucosamine, sarcosine, serinol, 2-amino-1,3-propanediol, 3-amino-1,2-propanediol, 4-amino-1,2,3-butanetriol, or a salt with a quarternary ammonium ion having 1 to 20 carbon atoms, such as tetramethylammonium, tetraethylammonium, tetra(n-propyl)ammonium, tetra(n-butyl)ammonium, N-benzyl-N,N,N-trimethylammonium, choline or benzalkonium.

Those skilled in the art will further recognise that it is possible for acid salts of the claimed compounds to be prepared by reaction of the compounds with the appropriate inorganic or organic acid via any of a number of known methods. Alternatively, alkali and alkaline earth metal salts of acidic compounds of the present invention are prepared by reacting the compounds of the present invention with the appropriate base via a variety of known methods.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae relating to salts, such as “hydrochloride”, “trifluoroacetate”, “sodium salt”, or “x HCl”, “x CF₃COOH”, “x Na⁺”, for example, mean a salt form, the stoichiometry of which salt form not being specified.

This applies analogously to cases in which synthesis intermediates or example compounds or salts thereof have been obtained, by the preparation and/or purification processes described, as solvates, such as hydrates, with (if defined) unknown stoichiometric composition.

Furthermore, the present invention includes all possible crystalline forms, or polymorphs, of the compounds of the present invention, either as single polymorph, or as a mixture of more than one polymorph, in any ratio.

Moreover, the present invention also includes prodrugs of the compounds according to the invention. The term “prodrugs” here designates compounds which themselves can be biologically active or inactive, but are converted (for example metabolically or hydrolytically) into compounds according to the invention during their residence time in the body.

In accordance with an alternate embodiment of the first aspect, the present invention covers compounds of the general formula (I), supra, in which:

C is the macrocyclic chelating agent Macropa below, where the substituent R is attached to any free carbon atom at the pyridine ring:

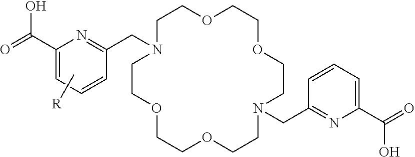

wherein R=NH₂ or CH₂CH₂COOH.

C can also be the macrocyclic chelating agent macropa below:

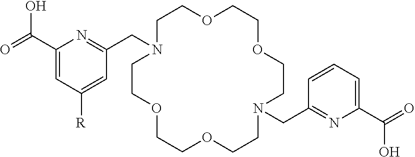

wherein R=NH₂ or CH₂CH₂COOH.

In accordance with a second embodiment of the first aspect, the present invention covers compounds of general formula (I), supra, in which:
C is the macrocyclic chelating agent macropa-NH₂ below:

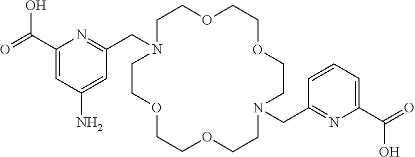 [SMILES: Nc1cc(CN2CCOCCOCCN(Cc3cccc(C(=O)O)n3)CCOCCOCC2)nc(C(=O)O)c1]

wherein either the amino substituent group or the carboxylic acid groups are used to form amide bonds with either L or V, n is 2, and V is a monoclonal antibody,
and stereoisomers, tautomers, N-oxides, hydrates, solvates, and salts thereof, and mixtures of same.

In accordance with a third embodiment of the first aspect, the present invention covers compounds of general formula (I), supra, in which:
C is the macrocyclic chelating agent macropa-NH₂ below:

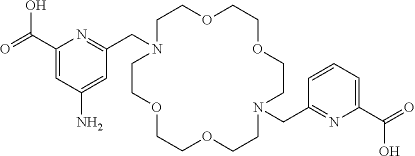 [SMILES: Nc1cc(CN2CCOCCOCCN(Cc3cccc(C(=O)O)n3)CCOCCOCC2)nc(C(=O)O)c1]

wherein either the amino substituent group or the carboxylic acid groups are used to form amide bonds with either L or V, n is 3, and V is a monoclonal antibody,
and stereoisomers, tautomers, N-oxides, hydrates, solvates, and salts thereof, and mixtures of same.

In accordance with a fourth embodiment of the first aspect, the present invention covers compounds of general formula (I), supra, in which:
C is the macrocyclic chelating agent macropa-NH₂ below:

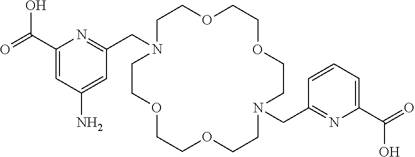 [SMILES: Nc1cc(CN2CCOCCOCCN(Cc3cccc(C(=O)O)n3)CCOCCOCC2)nc(C(=O)O)c1]

wherein either the amino substituent group or the carboxylic acid groups are used to form amide bonds with either L or V, n is 4, and V is a monoclonal antibody,
and stereoisomers, tautomers, N-oxides, hydrates, solvates, and salts thereof, and mixtures of same.

In accordance with a fifth embodiment of the first aspect, the present invention covers compounds of general formula (I), supra, in which:
C is the macrocyclic chelating agent macropa-NH₂ below:

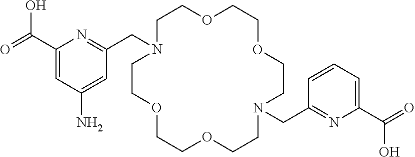 [SMILES: Nc1cc(CN2CCOCCOCCN(Cc3cccc(C(=O)O)n3)CCOCCOCC2)nc(C(=O)O)c1]

wherein either the amino substituent group or the carboxylic acid groups are used to form amide bonds with either L or V, n is greater then 4 but less than 20, and V is a monoclonal antibody, and stereoisomers, tautomers, N-oxides, hydrates, solvates, and salts thereof, and mixtures of same.

In a further embodiment of the first aspect, the present invention covers compounds of formula (I), supra, in which:
C is the macrocyclic chelating agent macropa-NH₂ below:

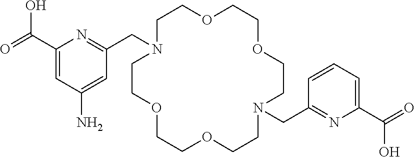

wherein n is 4, and V is a monoclonal antibody, and C is linked via a tetraamino backbone modified with a diglycolic acid spacer,
and stereoisomers, tautomers, N-oxides, hydrates, solvates, and salts thereof, and mixtures of same.

In a further embodiment of the first aspect, the present invention covers compounds of formula (I), supra, in which:
C can also be the macrocyclic chelating agent macropa below:

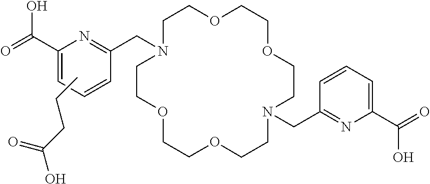

wherein n is 4, and V is a monoclonal antibody, and C is linked via a a propionic acid spacer to a tetraamino backbone,
and stereoisomers, tautomers, N-oxides, hydrates, solvates, and salts thereof, and mixtures of same.

In a further embodiment of the first aspect, the present invention covers compounds of formula (I), supra, in which:
C is the macrocyclic chelating agent macropa below:

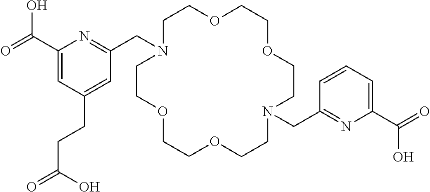

wherein n is 4, and V is a monoclonal antibody, and C is linked via a a propionic acid spacer to a tetraamino backbone,
and stereoisomers, tautomers, N-oxides, hydrates, solvates, and salts thereof, and mixtures of same.

The original priority application claimed the following:

1. A compound of general formula (I):

[(C)n-L]-(V)m (I),
in which: C represents the macrocyclic chelating agent macropa, L represents a multi-functional linker moiety comprising multiple functional groups for the covalent attachment of C, and V is a tissue-targeting moiety, and wherein n>1 and m is from 1 to 5, or a stereoisomer, a tautomer, an N-oxide, a hydrate, a solvate, or a salt thereof, or a mixture of same.

2. The compound according to claim 1, wherein the compound further comprises an alpha-emitting radioisotope or a stereoisomer, a tautomer, an N-oxide, a hydrate, a solvate, or a salt thereof, or a mixture of same.

3. The compound according to claim 2, wherein the alpha-emitting radioisotope is selected from the group consisting of radium-223, radium-224, Bi-212, Bi-213 and actinium-225 or a stereoisomer, a tautomer, an N-oxide, a hydrate, a solvate, or a salt thereof, or a mixture of same.

4. The compound according to claim 1, 2 or 3, wherein the tissue-targeting moiety is a monoclonal antibody or a stereoisomer, a tautomer, an N-oxide, a hydrate, a solvate, or a salt thereof, or a mixture of same.

5. The compound according to claim 1, 2, 3 or 4, wherein:
C is the macrocyclic chelating agent macropa below:

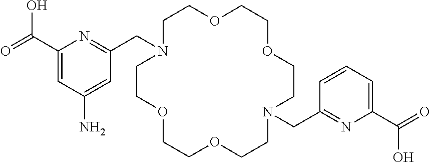 [SMILES: Nc1cc(CN2CCOCCOCCN(Cc3cccc(C(=O)O)n3)CCOCCOCC2)nc(C(=O)O)c1]

wherein either the amino substituent group or the carboxylic acid groups are used to form amide bonds with either L or V, n is 2, and V is a monoclonal antibody, or a stereoisomer, a tautomer, an N-oxide, a hydrate, a solvate, or a salt thereof, or a mixture of same.

6. The compound according to claim 1, 2, 3 or 4, wherein:
C is the macrocyclic chelating agent macropa below:

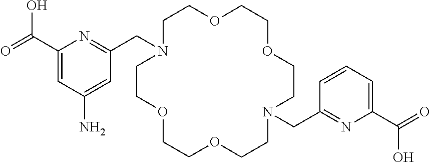 [SMILES: Nc1cc(CN2CCOCCOCCN(Cc3cccc(C(=O)O)n3)CCOCCOCC2)nc(C(=O)O)c1]

wherein either the amino substituent group or the carboxylic acid groups are used to form amide bonds with either L or V, n is 3, and V is a monoclonal antibody, or a stereoisomer, a tautomer, an N-oxide, a hydrate, a solvate, or a salt thereof, or a mixture of same.

7. The compound according to claim 1, 2, 3 or 4, wherein:
C is the macrocyclic chelating agent macropa below:

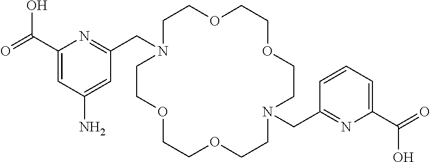 [SMILES: Nc1cc(CN2CCOCCOCCN(Cc3cccc(C(=O)O)n3)CCOCCOCC2)nc(C(=O)O)c1]

wherein either the amino substituent group orthe carboxylic acid groups are used to form amide bonds with either L or V, n is 4, and V is a monoclonal antibody, or a stereoisomer, a tautomer, an N-oxide, a hydrate, a solvate, or a salt thereof, or a mixture of same.

8. A method of preparing a compound of general formula (I) according to any one of claims 1 to 7, said method comprising the step of allowing an intermediate compound of general formula (II):

[(X)p′-C]n-L (II),
in which C, L, n and m and m are as defined for the compound of general formula (I) according to any one of claims 1 to 7,
to react with V;
in which V is as defined forthe compound of general formula (I) according to any one of claims 1 to 7,
thereby giving a compound of general formula (I):

[(C)n-L]-(V)m (I),
in which C, L, V, n and and m are as defined forthe compound of general formula (I) according to any one of claims 1 to 7.

9. A compound of general formula (I) according to any one of claims 1 to 7 for use in the treatment or prophylaxis of a disease.

10. A pharmaceutical composition comprising a compound of general formula (I) according to any one of claims 1 to 7 and one or more pharmaceutically acceptable excipients.

11. A pharmaceutical combination comprising:
one or more first active ingredients, in particular compounds of general formula (I) according to any one of claims 1 to 7, and
one or more further active ingredients, in particular anti-cancer agents.

12. Use of a compound of general formula (I) according to any one of claims 1 to 7 for the treatment or prophylaxis of a disease.

13. Use of a compound of general formula (I) according to any one of claims 1 to 7 for the preparation of a medicament for the treatment or prophylaxis of a disease.

14. Use according to claim 9, 12 or 13, wherein the disease is a hyperproliferative disorder, such as a oncological disorder, for example.

In a particular further embodiment of the first aspect, the present invention covers combinations of two or more of the above mentioned embodiments under the heading “further embodiments of the first aspect of the present invention”.

The present invention covers any sub-combination within any embodiment or aspect of the present invention of compounds of general formula (I), supra.

The present invention covers the compounds of general formula (I) which are disclosed in the Example Section of this text, infra.

The compounds according to the invention of general formula (I) can be prepared according to the following schemes 1 and 2. The schemes and procedures described below illustrate synthetic routes to the compounds of general formula (I) of the invention and are not intended to be limiting. It is clear to the person skilled in the art that the order of transformations as exemplified in schemes 1 and 2 can be modified in various ways. The order of transformations exemplified in these schemes is therefore not intendedto be limiting. In addition, interconversion of any of the substituents, can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs.

Two routes for the preparation of compounds of general formula (I) are described in schemes 1 and 2.

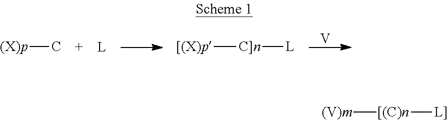

Scheme 1: Route for the preparation of compounds of general formula (I) in which C, L, V, n and and m have the meaning as given for general formula (I), supra, and X is a functional group or more preferably a reactive functional group, and p is 1-10 and p′ is 1-10, more preferably p and p′ are 1-4.

The chelators C may be activated with a reactive functional group X such as e.g. an NHS ester, a TFP ester, an HOBt ester, an HOAt ester or NSC group for further conjugation to L being e.g. an poly-amine containing backbone. The formation of resulting amide bonds or thiourea bonds between C and L can be done in aqueous or organic solvents at pH between 7 and 11 at room temperature or elevated temperatures. Isolation of intermediates and products may be carried out with e.g. preparative HPLC or other known separation techniques. Conjugation of multimeric chelators of general formula (II),

[(X)p′-C]n-L (II)

to targeting moiety V can be effectuated by X being a reactive functional group such as an NHS ester, a TFP ester or a NSC group which forms amide bonds or thiourea bonds with V, e.g. conjugation to lysine side chain amino groups of an antibody, to make a compound of genera formula (I) as defined supra.

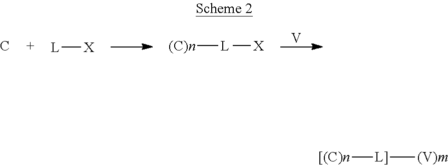

Scheme 2: Route for the preparation of compounds of general formula (I) in which C, L, V, n and and m have the meaning as given for general formula (I), supra, and X is a reactive functional group.

The chelators C may be conjugated to L being e.g. an poly-amine containing backbone containing a protected reactive functional group. The formation of resulting amide bonds or thiourea bonds between C and L can be done in aqueous or organic solvents at pH between 7 and 11 at room temperature or elevated temperatures. Isolation of intermediates and products may be carried out with e.g. preparative HPLC or other known separation techniques. Conjugation of multimeric chelators of general formula (III),

(C)n-L-X (III)

to targeting moiety V can be effectuated by X being a reactive functional group such as an NHS ester, a TFP ester or a NSC group which forms amide bonds or thiourea bonds with V, e.g. conjugation to lysine side chain amino groups of an antibody, to make a compound of genera formula (I) as defined supra. Specific examples are described in the Experimental Section.

The present invention covers the intermediate compounds defined by formula (II) and formula (III) which are disclosed in the Example Section of this text, infra.

The present invention covers any sub-combination within any embodiment or aspect of the present invention of intermediate compounds. of general formula (II) and (III), supra.

The compounds of general formula (I) of the present invention can be converted to any salt, preferably pharmaceutically acceptable salts, as described herein, by any method which is known to the person skilled in the art. Similarly, any salt of a compound of general formula (I) of the present invention can be converted into the free compound, by any method which is known to the person skilled in the art.

Compounds of general formula (I) of the present invention demonstrate a valuable pharmacological spectrum of action and pharmacokinetic profile, both of which could not have been predicted. Compounds of the present invention have surprisingly been found to effectively inhibit target and it is possible therefore that said compounds be used for the treatment or prophylaxis of diseases, preferably hyperproliferative disorders in humans and animals.

Compounds of the present invention can be utilized to inhibit, block, reduce, decrease, etc., cell proliferation and/or cell division, and/or produce apoptosis. This method comprises administering to a mammal in need thereof, including a human, an amount of a compound of general formula (I) of the present invention, or a pharmaceutically acceptable salt, isomer, polymorph, metabolite, hydrate, solvate or ester thereof, which is effective to treat the disorder.

Hyperproliferative disorders include, but are not limited to, for example : psoriasis, keloids, and other hyperplasias affecting the skin, benign prostate hyperplasia (BPH), solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias.

Examples of breast cancers include, but are not limited to, invasive ductal carcinoma, invasive lobular carcinoma, ductal carcinoma in situ, and lobular carcinoma in situ.

Examples of cancers of the respiratory tract include, but are not limited to, small-cell and non-small-cell lung carcinoma, as well as bronchial adenoma and pleuropulmonary blastoma.

Examples of brain cancers include, but are not limited to, brain stem and hypophtalmic glioma, cerebellar and cerebral astrocytoma, medulloblastoma, ependymoma, as well as neuroectodermal and pineal tumour.

Tumours of the male reproductive organs include, but are not limited to, prostate and testicula cancer.

Tumours of the female reproductive organs include, but are not limited to, endometrial, cervical, ovarian, vaginal, and vulvar cancer, as well as sarcoma of the uterus.

Tumours of the digestive tract include, but are not limited to, anal, colon, colorectal, oesophageal, gallbladder, gastric, pancreatic, rectal, small-intestine, and salivary gland cancers.

Tumours of the urinary tract include, but are not limited to, bladder, penile, kidney, renal pelvis, ureter, urethral and human papillary renal cancers.

Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to, squamous cell carcinoma, Kaposi's sarcoma, malignant melanoma, Merkel cell skin cancer, and non-melanoma skin cancer.

Head-and-neck cancers include, but are not limited to, laryngeal, hypopharyngeal, nasopharyngeal, oropharyngeal cancer, lip and oral cavity cancer and squamous cell.

Lymphomas include, but are not limited to, AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to, sarcoma of the soft tissue, osteosarcoma, malignant fibrous histiocytoma, lymphosarcoma, and rhabdomyosarcoma.

Leukemias include, but are not limited to, acute myeloid leukemia, acute lymphoblastic leukemia, chronic lymphocytic leukemia, chronic myelogenous leukemia, and hairy cell leukemia.

The present invention also provides methods of treating angiogenic disorders including diseases associated with excessive and/or abnormal angiogenesis.

Inappropriate and ectopic expression of angiogenesis can be deleterious to an organism. A number of pathological conditions are associated with the growth of extraneous blood vessels. These include, for example, diabetic retinopathy, ischemic retinal-vein occlusion, and retinopathy of prematurity [Aiello et al., New Engl. J. Med., 1994, 331, 1480 ; Peer et al., Lab. Invest., 1995, 72, 638], age-related macular degeneration (AMD) [Lopez et al., Invest Opththalmol. Vis. Sci., 1996, 37, 855], neovascular glaucoma, psoriasis, retrolental fibroplasias, angiofibroma, inflammation, rheumatoid arthritis (RA), restenosis, in-stent restenosis, vascula graft restenosis, etc. In addition, the increased blood supply associated with cancerous and neoplastic tissue, encourages growth, leading to rapid tumour enlargement and metastasis. Moreover, the growth of new blood and lymph vessels in a tumour provides an escape route for renegade cells, encouraging metastasis and the consequence spread of the cancer. Thus, compounds of general formula (I) of the present invention can be utilized to treat and/or prevent any of the aforementioned angiogenesis disorders, for example by inhibiting and/or reducing blood vessel formation; by inhibiting, blocking, reducing, decreasing, etc. endothelial cell proliferation, or other types involved in angiogenesis, as well as causing cell death or apoptosis of such cell types.

These disorders have been well characterized in humans, but also exist with a similar etiology in other mammals, and can be treated by administering pharmaceutical compositions of the present invention.

The term “treating” or “treatment” as stated throughout this document is used conventionally, for example the management or care of a subject for the purpose of combating, alleviating, reducing, relieving, improving the condition of a disease or disorder, such as a carcinoma.

Preferably, the targeted alpha therapy of the present invention is for the treatment of Non-Hodgkin's Lymphoma or B-cell neoplasms, breast, colorectal, endometrial, gastric, acute myeloid leukemia, prostate or brain, mesothelioma, ovarian, lung or pancreatic cancer. Typically, the combination therapy of the present invention will be used in the treatment of ovarian cancer, breast cancer, gastric cancer, lung cancer, colorectal cancer or Acute Myeloid Leukaemia.

Generally, the use of chemotherapeutic agents and/or anti-cancer agents in combination with a compound or pharmaceutical composition of the present invention will serve to:
1. yield better efficacy in reducing the growth of a tumour or even eliminate the tumour as compared to administration of either agent alone,
2. provide for the administration of lesser amounts of the administered chemotherapeutic agents,
3. provide for a chemotherapeutic treatment that is well tolerated in the patient with fewer deleterious pharmacological complications than observed with single agent chemotherapies and certain other combined therapies,
4. provide for treating a broader spectrum of different cancer types in mammals, especially humans,
5. provide for a higher response rate among treated patients,
6. provide for a longer survival time among treated patients compared to standard chemotherapy treatments,
7. provide a longer time for tumour progression, and/or
8. yield efficacy and tolerability results at least as good as those of the agents used alone, compared to known instances where other cancer agent combinations produce antagonistic effects.

In addition, the compounds of general formula (I) of the present invention can also be used in combination with radiotherapy and/or surgical intervention.

In a further embodiment of the present invention, the compounds of general formula (I) of the present invention may be used to sensitize a cell to radiation, i.e. treatment of a cell with a compound of the present invention priorto radiation treatment of the cell renders the cell more susceptible to DNA damage and cell death than the cell would be in the absence of any treatment with a compound of the present invention. In one aspect, the cell is treated with at least one compound of general formula (I) of the present invention.

Thus, the present invention also provides a method of killing a cell, wherein a cell is administered one or more compounds of the present invention in combination with conventional radiation therapy.

The present invention also provides a method of rendering a cell more susceptible to cell death, wherein the cell is treated with one or more compounds of general formula (I) of the present invention prior to the treatment of the cell to cause or induce cell death. In one aspect, after the cell is treated with one or more compounds of general formula (I) of the present invention, the cell is treated with at least one compound, or at least one method, or a combination thereof, in order to cause DNA damage for the purpose of inhibiting the function of the normal cell or killing the cell.

In other embodiments of the present invention, a cell is killed by treating the cell with at least one DNA damaging agent, i.e. after treating a cell with one or more compounds of genera formula (I) of the present invention to sensitize the cell to cell death, the cell is treated with at least one DNA damaging agent to kill the cell. DNA damaging agents useful in the present invention include, but are not limited to, chemotherapeutic agents (e.g. cis platin), ionizing radiation (X-rays, ultraviolet radiation), carcinogenic agents, and mutagenic agents.

In other embodiments, a cell is killed by treating the cell with at least one method to cause or induce DNA damage. Such methods include, but are not limited to, activation of a cell signalling pathway that results in DNA damage when the pathway is activated, inhibiting of a cell signalling pathway that results in DNA damage when the pathway is inhibited, and inducing a biochemical change in a cell, wherein the change results in DNA damage. By way of a non-limiting example, a DNA repair pathway in a cell can be inhibited, thereby preventing the repair of DNA damage and resulting in an abnormal accumulation of DNA damage in a cell.

In one aspect of the invention, a compound of general formula (I) of the present invention is administered to a cell prior to the radiation or other induction of DNA damage in the cell. In another aspect of the invention, a compound of general formula (I) of the present invention is administered to a cell concomitantly with the radiation or other induction of DNA damage in the cell. In yet another aspect of the invention, a compound of general formula (I) of the present invention is administered to a cell immediately after radiation or other induction of DNA damage in the cell has begun.

In another aspect, the cell is in vitro. In another embodiment, the cell is in vivo.

In accordance with a further aspect, the present invention covers compounds of general formula (I), as described supra, or stereoisomers, tautomers, N-oxides, hydrates, solvates, and salts thereof, particularly pharmaceutically acceptable salts thereof, or mixtures of same, for use in the treatment or prophylaxis of diseases, in particular hyperproliferative disorders.

The pharmaceutical activity of the compounds according to the invention can be explained by their activity as mechanism.

In accordance with a further aspect, the present invention covers the use of compounds of general formula (I), as described supra, or stereoisomers, tautomers, N-oxides, hydrates, solvates, and salts thereof, particularly pharmaceutically acceptable salts thereof, or mixtures of same, for the treatment or prophylaxis of diseases, in particular hyperproliferative disorders, particularly oncological disorders.

In accordance with a further aspect, the present invention covers the use of a compound of formula (I), described supra, ora stereoisomer, a tautomer, an N-oxide, a hydrate, a solvate, or a salt thereof, particularly a pharmaceutically acceptable salt thereof, or a mixture of same, for the prophylaxis or treatment of diseases, in particular hyperproliferative disorders, particularly oncological disorders.

In accordance with a further aspect, the present invention covers the use of compounds of general formula (I), as described supra, or stereoisomers, tautomers, N-oxides, hydrates, solvates, and salts thereof, particularly pharmaceutically acceptable salts thereof, or mixtures of same, in a method of treatment or prophylaxis of diseases, in particular hyperproliferative disorders, particularly oncological disorders.

In accordance with a further aspect, the present invention covers use of a compound of genera formula (I), as described supra, or stereoisomers, tautomers, N-oxides, hydrates, solvates, aid salts thereof, particularly pharmaceutically acceptable salts thereof, or mixtures of same, for the preparation of a pharmaceutical composition, preferably a medicament, for the prophylaxis or treatment of diseases, in particular hyperproliferative disorders, particularly oncological disorders.

In accordance with a further aspect, the present invention covers a method of treatment or prophylaxis of diseases, in particular hyperproliferative disorders, particularly oncological disorders, using an effective amount of a compound of general formula (I), as described supra, or stereoisomers, tautomers, N-oxides, hydrates, solvates, and salts thereof, particularly pharmaceutically acceptable salts thereof, or mixtures of same.

In accordance with a further aspect, the present invention covers pharmaceutical compositions, in particular a medicament, comprising a compound of general formula (I), as described supra, or a stereoisomer, a tautomer, an N-oxide, a hydrate, a solvate, a salt thereof, particularly a pharmaceutically acceptable salt, ora mixture of same, and one or more excipients), in particular one or more pharmaceutically acceptable excipient(s). Conventional procedures for preparing such pharmaceutical compositions in appropriate dosage forms can be utilized.

The present invention furthermore covers pharmaceutical compositions, in particular medicaments, which comprise at least one compound according to the invention, conventionally together with one or more pharmaceutically suitable excipients, and to their use for the above mentioned purposes.

It is possible for the compounds according to the invention to have systemic and/or local activity. For this purpose, they can be administered in a suitable manner, such as, for example, via the, parenteral.

For these administration routes, it is possible for the compounds according to the invention to be administered in suitable administration forms.

Parenteral administration can be effected with avoidance of an absorption step (for example intravenous, intraarterial, intracardial, intraspinal or intralumbal). Administration forms which are suitable for parenteral administration are, inter alia, preparations for injection and infusion in the form of solutions, suspensions, emulsions, lyophylisates or sterile powders.

The compounds according to the invention can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (forexample highly-disperse silicas),
viscosity-increasing agents, gel formers, thickeners and/or binders (for example polyvinylpyrrolidone, methylcellulose, hydroxypropylmethylcellulose, hydroxypropyl-cellulose, carboxymethylcellulose-sodium, starch, carbomers, polyacrylicacids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropyl-methylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to a pharmaceutical composition which comprise at least one compound according to the invention, conventionally together with one or more pharmaceutically suitable excipient(s), and to their use according to the present invention.

In accordance with another aspect, the present invention covers pharmaceutical combinations, in particular medicaments, comprising at least one compound of general formula (I) of the present invention and at least one or more further active ingredients, in particular for the treatment and/or prophylaxis of a hyperproliferative disorder.

Particularly, the present invention covers a pharmaceutical combination, which comprises:
one or more first active ingredients, in particular compounds of general formula (I) as defined supra, and
one or more further active ingredients, in particular for the treatment of hyperproliferative disorder.

The term “combination” in the present invention is used as known to persons skilled in the art, it being possible for said combination to be a fixed combination, a non-fixed combination or a kit-of-parts.

A “fixed combination” in the present invention is used as known to persons skilled in the art and is defined as a combination wherein, for example, a first active ingredient, such as one or more compounds of general formula (I) of the present invention, and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” in the present invention is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

The compounds of the present invention can be administered as the sole pharmaceutical agent or in combination with one or more other pharmaceutically active ingredients where the combination causes no unacceptable adverse effects. The present invention also covers such pharmaceutical combinations. For example, the compounds of the present invention can be combined with known anti-cancer agents.

Examples of anti-cancer agents include:
131 I-chTNT, abarelix, abemaciclib, abiraterone, acalabrutinib, aclarubicin, adalimumab, ado-trastuzumab emtansine, afatinib, aflibercept, aldesleukin, alectinib, alemtuzumab, alendronic acid, alitretinoin, alpharadin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, apalutamide, aprepitant, arcitumomab, arglabin, arsenic trioxide, asparaginase, atezolizumab, avelumab, axicabtagene ciloleucel, axitinib, azacitidine, basiliximab, belotecan, bendamustine, besilesomab, belinostat, bevacizumab, bexarotene, bicalutamide, bisantrene, bleomycin, blinatumomab, bortezomib, bosutinib, buserelin, brentuximab vedotin, brigatinib, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, cemiplimab, ceritinib, cetuximab, chlorambucil, chlormadinone, chlormethine, cidofovir, cinacalcet, cisplatin, cladribine, clodronic acid, clofarabine, cobimetinib, copanlisib , crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, durvalumab, eculizumab, edrecolomab, elliptinium acetate, elotuzumab, eltrombopag, enasidenib, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, inotuzumab ozogamicin, interferon alfa, interferon beta, interferon gamma, iobitridol, iobenguane (123I), iomeprol, ipilimumab, irinotecan, Itraconazole, ixabepilone, ixazomib, lanreotide, lansoprazole, lapatinib, lasocholine, lenalidomide, lenvatinib, lenograstim, lentinan, letrozole, leuprorelin, levamisole, levonorgestrel, levothyroxine sodium, lisuride, lobaplatin, lomustine, lonidamine, lutetium Lu 177 dotatate, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, midostaurin, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, mvasi, nabilone, nabiximols, nafarelin, naloxone+pentazocine, naltrexone, nartograstim, necitumumab, nedaplatin, nelarabine, neratinib, neridronic acid, netupitant/palonosetron, nivolumab, pentetreotide, nilotinib, nilutamide, nimorazole, nimotuzumab, nimustine, nintedanib, niraparib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pembrolizumab, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib , regorafenib, ribociclib, risedronic acid, rhenium-186 etidronate, rituximab, rogaratinib, rolapitant, romidepsin, romiplostim, romurtide, rucaparib, samarium (153Sm) lexidronam, sargramostim, sarilumab, satumomab, secretin, siltuximab, sipuleucel-T, sizofiren, sobuzoxane, sodium glycididazole, sonidegib, sorafenib, stanozolol, streptozocin, sunitinib, talaporf in, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporf in, temozolomide, temsirolimus, teniposide, testosterone, tetrofosmin, thalidomide, thiotepa, thymalfasin, thyrotropin alfa, tioguanine, tisagenlecleucel, tislelizumab, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib , valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

Based upon standard laboratory techniques known to evaluate compounds useful for the treatment of hyperproliferative disorders, by standard toxicity tests and by standard pharmacological assays for the determination of treatment of the conditions identified above in mammals, and by comparison of these results with the results of known active ingredients or medicaments that are used to treat these conditions, the effective dosage of the compounds of the present invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 10 mg/kg body weight per day, and preferably from about 0.01 mg/kg to about 1 mg/kg body weight per day. Clinically useful dosing schedules will range from one to four times a month dosing to once every two to eight months dosing. In addition, it is possible for “drug holidays”, in which a patient is not dosed with a drug for a certain period of time, to be beneficial to the overall balance between pharmacological effect and tolerability.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

### EXPERIMENTAL SECTION

Chemical names were generated using the ACD/Name software from ACD/Labs. In some cases generally accepted names of commercially available reagents were used in place of ACD/Name generated names.

The following table 1 lists the abbreviations used in this paragraph and in the Examples section as far as they are not explained within the text body. Other abbreviations have their meanings customary per se to the skilled person.

### Table 1: Abbreviations

The following table lists the abbreviations used herein.
²²³Ra radium-223
²²⁵Ac actinium-225
Ac-225 actinium-225
ACC antibody-chelator conjugate
ACN acetonitrile
Bn benzyl
CAR chelator-to-antibody ratio
DCC N,N′-dicyclohexylcarbodiimide
DCM dichloromethane
DIPEA N,N-diisopropylethylamine
DMA N,N-dimethylacrylamide
DMSO dimethyl sulf oxide
DOTA 1,4,7, 10-tetraazacyclododecane-1,4,7, 10-tetraacetic acid
DSS Sodium trimethylsilylpropanesulfonate
ESI electrospray ionization
EtOAc Ethyl acetate
EtOH ethanol
FA formic acid
FPLC fast protein liquid chromatography
HCl hydrochloric acid
HPGe high purity germanium
HPLC high performance liquid chromatography
iTLC instant thin layer chromatography
IRF immunoreactive fraction
Lys lysine
mAb monoclonal antibody
min minutes
MS mass spectrometry
NaCl sodium chloride
NMP N-methyl-2-pyrrolidone
nm nanometer
nmol nanomol
NMR nuclear magnetic resonance
PBS phosphate buffered saline
PEG poly(ethylene glycol)
PLT platelets
PyAOP (7-Azabenzotriazol-1-yloxy)tripyrrolidinophosphonium hexafluorophosphate
Ra-223 radium-223
RAC radioactive concentration
RCP radiochemical purity
SEC size exclusion chromatography
tBu tert-butyl
TFA trifluoroacetic acid
TFP 2,3,5,6-tetrafluorophenol
TOF time of flight
UPLC ultra performance liquid chromatography
WBC white blood cells

The various aspects of the invention described in this application are illustrated by the following examples which are not meant to limit the invention in any way.

The example testing experiments described herein serve to illustrate the present invention aid the invention is not limited to the examples given.

### Experimental Section—General Part

All reagents, for which the synthesis is not described in the experimental part, are either commercially available, or are known compounds or may be formed from known compounds by known methods by a person skilled in the art.

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using for example prepacked silica gel cartridges, e.g. Biotage SNAP cartidges KP-Sil® or KP-NH® in combination with a Biotage autopurifier system (SP4® or Isolera Four®) and eluents such as gradients of hexane/ethyl acetate or DCM/methanol. In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid or aqueous ammonia.

In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc.) of a compound of the present invention as isolated and as described herein is not necessarily the only form in which said compound can be applied to a biological assay in orderto quantify the specificbiological activity.

NMR peak forms are stated as they appear in the spectra, possible higher order effects have not been considered.

The ¹H-NMR data of selected compounds are listed in the form of ¹H-NMR peaklists. Therein, for each signal peak the δ value in ppm is given, followed by the signal intensity, reported in round brackets. The δ value-signal intensity pairs from different peaks are separated by commas. Therefore, a peaklist is described by the general form: δ₁ (intensity₁), δ₂ (intensity₂), . . . , δᵢ (intensityᵢ), . . . , δₙ (intensityₙ).

The intensity of a sharp signal correlates with the height (in cm) of the signal in a printed NMR spectrum. When compared with other signals, this data can be correlated to the real ratios of the signal intensities. In the case of broad signals, more than one peak, or the center of the signal along with their relative intensity, compared to the most intense signal displayed in the spectrum, are shown. A ¹H-NMR peaklist is similar to a classical ¹H-NMR readout, and thus usually contains all the peaks listed in a classical NMR interpretation. Moreover, similar to classical ¹H-NMR printouts, peaklists can show solvent signals, signals derived from stereoisomers of the particular target compound, peaks of impurities, ¹³C satellite peaks, and/or spinning sidebands. The peaks of stereoisomers, and/or peaks of impurities are typically displayed with a lower intensity compared to the peaks of the target compound (e.g., with a purity of >90%). Such stereoisomers and/or impurities may be typical for the particular manufacturing process, and therefore their peaks may help to identify a reproduction of the manufacturing process on the basis of “by-product fingerprints”. An expert who calculates the peaks of the target compound by known methods (MestReC, ACD simulation, or by use of empirically evaluated expectation values), can isolate the peaks of the target compound as required, optionally using additional intensity filters. Such an operation would be similar to peak-picking in classical ¹H-NMR interpretation. A detailed description of the reporting of NMR data in the form of peaklists can be found in the publication “Citation of NMR Peaklist Data within Patent Applications” (cf. http://www.researchdisclosure.com/searching-disclosures, Research Disclosure Database Number 605005, 2014, 1 Aug. 2014). In the peak picking routine, as described in the Research Disclosure Database Number 605005, the parameter “MinimumHeight” can be adjusted between 1% and 4%. However, depending on the chemical structure and/or depending on the concentration of the measured compound it may be reasonable to set the parameter “MinimumHeight” <1%.

### UPLC-MS Standard Procedures

Analytical UPLC-MS was performed as described below. The masses (m/z) are reported from the positive mode electrospray ionisation (ESI+) unless the negative mode is indicated (ESI−). In most of the cases method 1 is used. If not, it is indicated.

### Method 1

Instrument: Waters Acquity UPLC-MS XEVO; Column: Acquity UPLC BEH C18 1.7 50×2.1 mm; Eluent A: water+0.1% TFA, Eluent B: acetonitrile;; Flow rate 0.5 mL/min; Temperature: Ambient; Injection: 10 μL; DAD scan: 210-400 nm;

### Method 2

Instrument: SHIMADZU LCMS-2020 SingleQuad; Column: Chromolith@Flash RP-18E 25-2 MM; eluent A: water+0.0375 vol % trifluoroacetic acid, eluent B: acetonitrile+0.01875 vol % trifluoroacetic acid; gradient: 0-0.8 min, 5-95% B, 0.8-1.2 min 95% B; flow 1.5 ml/min; temperature: 50° C.; FDA: 220 nm & 254 nm.

### Experimental Section—Intermediates

### Intermediate 1

tert-butyl N-[(5S)-6-[2-[3-[bis[2-[[(2S)-2,6-bis(tert-butoxycarbonylamino)hexanoyl]amino]ethyl]amino]propyl-[2-[[(2S)-2,6-bis(tert-butoxycarbonylamino)hexanoyl]amino]ethyl]amino]ethylamino]-5-(tert-butoxycarbonylamino)-6-oxo-hexyl]carbamate

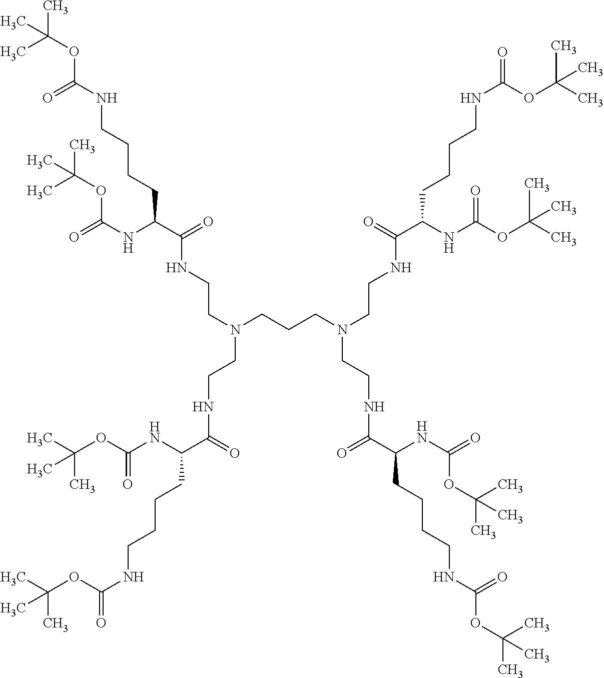 [SMILES: CC(C)(C)OC(=O)NCCCC[C@H](NC(=O)OC(C)(C)C)C(=O)NCCN(CCCN(CCNC(=O)[C@H](CCCCNC(=O)OC(C)(C)C)NC(=O)OC(C)(C)C)CCNC(=O)[C@H](CCCCNC(=O)OC(C)(C)C)NC(=O)OC(C)(C)C)CCNC(=O)[C@H](CCCCNC(=O)OC(C)(C)C)NC(=O)OC(C)(C)C]

A solution of L-lysine (1.47 g) in water/THF (50 mL was cooled in an ice-water bath and NaHCO₃ (2.52 g) and Boc anhydride (10.52 g) was added. The cooling bath was removed afterwards aid solution stirred at room temperature for 24 hrs. THF was evaporated under reduced pressure, 10% citric acid (aq) was added to obtain pH 3 and the mixture was extracted with DCM (2×100 mL), washed with water (50 mL) and brine (50 mL), dried (Na₂SO₄), filtered and concentrated under reduced pressure. Flash chromatography on silica gel eluting with DCM:MeOH (90:10) afforded 3.0 g (86%) of Boc-L-Lys(Boc)-OH as a colorless sticky solid.

To a mixture of N,N,N′,N′-tetrakis(2-aminoethyl)propane-1,3-diamine (92.9 mg, [142745-40-2]) and Boc-L-Lys(Boc)-OH (652.3 mg) in dry DMF (5 mL) was added HBTU (714 mg) and triethylamine (530 mL). The reaction mixture was stirred at room temperature for 7 days. The reaction mixture was concentrated underreduced pressure. The residuewas dissolved in EtOAc (100 mL), washed with 1M HCl (aq) (25 mL) and Na₂CO₃ (sat) (aq) (25 mL), dried (Na₂SO₄), filtered and concentrated under reduced pressure. Flash chromatography on silica gel eluting with CH₂Cl₂:MeOH (95:5)-(90:10) afforded 393 mg of the target compound.

### Intermediate 2

(2S)-2,6-diamino-N-[2-[3-[bis[2-[[(2S)-2,6-diaminohexanoyl]amino]ethyl]amino]propyl-[2-[[(2S)-2,6-diaminohexanoyl]amino]ethyl]amino]ethyl]hexanamide

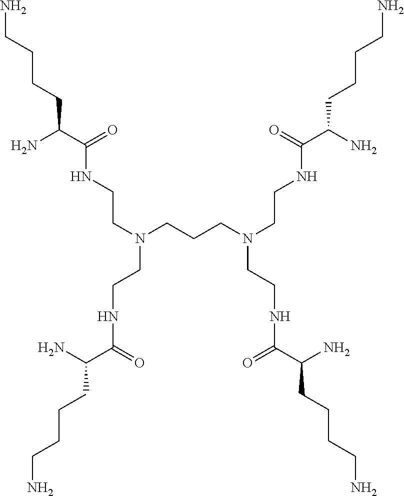 [SMILES: NCCCC[C@H](N)C(=O)NCCN(CCCN(CCNC(=O)[C@@H](N)CCCCN)CCNC(=O)[C@@H](N)CCCCN)CCNC(=O)[C@@H](N)CCCCN]

tert-butyl N-[(5S)-6-[2-[3-[bis[2-[[(2S)-2,6-bis(tert-butoxycarbonylamino)hexanoyl]amino]ethyl]amino]propyl-[2-[[(2S)-2,6-bis(tert-butoxycarbonylamino)hexanoyl]amino]ethyl]amino]ethylamino]-5-(tert-butoxycarbonylamino)-6-oxo-hexyl]carbamate (139 mg) is treated with 90% TFA/waterf or 30 min. Water (15 mL) is added and product lyophilised affording 219 mg of target compouns as TFA salt. The pure product was analyzed by analytical HPLC (gradient: 0-30% B over 2.5 min where A=water/0.1% TFA and B=ACN, flow rate: 0.5 mL/min, column: Waters Acquity BEH C18, 1.7 μm, 2.1×50 mm, detection: UV diode array, product retention time: 1.13 min). Further product characterization was carried out using electrospray mass spectrometry (MH⁺ 759.6, found m/z: 759.7).

### Intermediate 3 (M2)

2-[2-[[2-methoxycarbonyl-6-[[16-[(6-methoxycarbonyl-2-pyridyl)methyl]-1,4,10,13-tetraoxa-7,16-diazacyclooctadec-7-yl]methyl]-4-pyridyl]amino]-2-oxo-ethoxy]acetic acid

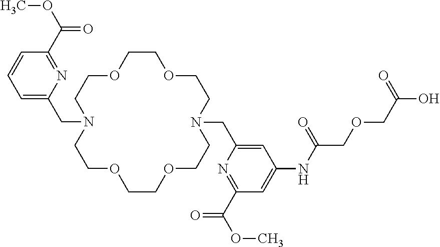 [SMILES: COC(=O)c1cccc(CN2CCOCCOCCN(Cc3cc(NC(=O)COCC(=O)O)cc(C(=O)OC)n3)CCOCCOCC2)n1]

Methyl 4-amino-6-[[16-[(6-methoxycarbonyl-2-pyridyl)methyl]-1,4,10,13-tetraoxa-7,16-diazacyclooctadec-7-yl]methyl]pyridine-2-carboxylate (81 mg, [2146091-22-5]) and diglycolic anhydride (163 mg) were dissolved in NMP (1 mL). DIPEA (245 μL) was added and solution kept at 40° C. over night. Solution was diluted with water/0.1% TFA (8 mL), adjusted to pH 3 with TFA (50 μL) and the product purified by preparative HPLC (column: Phenomenex Luna 5 μm C18(2) 100 Å, 250×50 mm; gradient: 10-50% B over 40 min where A=water/0.1% TFA and B=ACN; flow: 10 mL/min; detection: UV 214/254 nm) affording 67 mg (69% yield) of the target compound after freeze-drying. The pure product was analyzed by analytical HPLC (gradient: 10-50% B over 2.5 min where A=water/0.1% TFA and B=ACN, flow rate: 0.5 mL/min, column: Waters Acquity BEH C18, 1.7 μm, 2.1×50 mm, detection: UV diode array, product retention time: 1.19 min). Further product characterization was carried out using electrospray mass spectrometry (MH⁺692.3, found m/z: 692.3).

### Intermediate 4

Methyl 4-[(1E)-3-tert-butoxy-3-oxoprop-1-en-1-yl]-6-(hydroxymethyl)pyridine-2-carboxylate

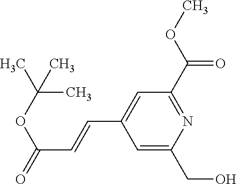 [SMILES: COC(=O)c1cc(/C=C/C(=O)OC(C)(C)C)cc(CO)n1]

To a mixture of methyl 4-bromo-6-(hydroxymethyl)pyridine-2-carboxylate (3.08 g, 12.5 mmol, [1842336-50-8]), tert-butyl prop-2-enoate (2.41 g, 18.8 mmol), tris-(o-tolyl)phosphine (381 mg, 1.25 mmol) and triethylamine (14 ml, 100 mmol) in acetonitrile (150 ml) was added palladium(II) acetate (141 mg, 0.626 mmol) at 25° C. under nitrogen atmosphere. After stirring at 80° C. for 16 hours under nitrogen atmosphere, the mixture was concentrated to give a residue. The residue was purified by flash column chromatography (petroleum ether/EtOAc=4:1 to 2:3) to give to give the target compound (3.37 g, 92% yield) as yellow oil.

¹H NMR (400 MHz, DMSO-d₆): δ[ppm]=8.15 (d, J=1.2 Hz, 1H), 7.92 (d, J=0.8 Hz, 1H), 7.65 (d, J=16.0 Hz, 1H), 6.81 (d, J=16.0 Hz, 1H), 5.58 (t, J=6.4 Hz, 2H), 4.62 (d, J=6.0 Hz, 1H), 3.89 (s, 3H), 1.49 (s, 9H).

### Intermediate 5

Methyl 4-(3-tert-butoxy-3-oxopropyl)-6-(hydroxymethyl)pyridine-2-carboxylate

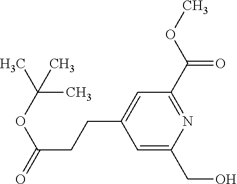 [SMILES: COC(=O)c1cc(CCC(=O)OC(C)(C)C)cc(CO)n1]

A mixture of methyl 4-[(1E)-3-tert-butoxy-3-oxoprop-1-en-1-yl]-6-(hydroxymethyppyridine-2-carboxylate (3.37 g, 11.5 mmol, Intermediate 4), palladium on activated carbon (337 mg, 10% purity, wet) in methanol (50 ml) was stirred at room temperature for 16 hours under hydrogen (15 psi). The mixture was filtered through a pad of celite and the filter cake was washed with methanol for three times. The filtrate was concentrated to give to give the target compound (3.00 g, 88% yield) as yellow oil.

¹H NMR (400 MHz, DMSO-d₆): δ[ppm]=7.79 (s, 1H), 7.58 (s, 1H), 5.54 (s, 1H), 4.58 (s, 2H), 3.86 (s, 3H), 2.93 (t, J=7.2 Hz, 2H), 2.60 (t, J=7.2 Hz, 2H), 1.35 (s, 9H).

### Intermediate 6

Methyl 4-(3-tert-butoxy-3-oxopropyl)-6-{[(methanesulfonyl)oxy]methyl}pyridine-2-carboxylate

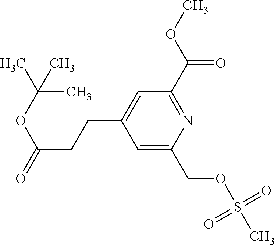 [SMILES: COC(=O)c1cc(CCC(=O)OC(C)(C)C)cc(COS(C)(=O)=O)n1]

To a mixture of methyl 4-(3-tert-butoxy-3-oxopropyl)-6-(hydroxymethyppyridine-2-carboxylate (3.60 g, 12.2 mmol, Intermediate 5) and triethylamine (5.1 ml, 37 mmol) in DCM (50 ml) was added methanesulfonyl chloride (1.68 g, 14.6 mmol) in drop-wise at 0° C. After stirring at 0° C. for 1 hour, the reaction mixture was quenched with water and extracted with dichloromethane. The combined organic phase was washed with brine, dried over anhydrous sodium sulfate, filtered and concentrated to give a residue. The residue was purified by flash column chromatography (petroleum ether/EtOAc=4:1 to 1:1) to give to give the target compound (3.10 g, 68% purity) as yellow oil.

¹H NMR (400 MHz, DMSO-d₆): δ[ppm]=7.94 (d, J=1.2 Hz, 1H), 7.65 (d, J=1.2 Hz, 1H), 5.34 (s, 2H), 3.88 (s, 3H), 3.32 (s, 3H), 2.96 (t, J=7.2 Hz, 2H), 2.63 (t, J=7.2 Hz, 2H), 1.35 (s, 9H).

### Intermediate 7

Methyl 6-(1,4,10,13-tetraoxa-7,16-diazacyclooctadec-7-ylmethyl)pyridine-2-carboxylate

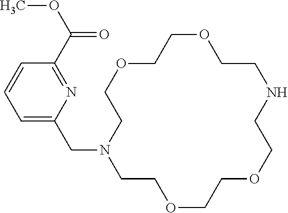 [SMILES: COC(=O)c1cccc(CN2CCOCCOCCNCCOCCOCC2)n1]

A mixture of 1,4,10,13-tetraoxa-7,16-diazacyclooctadecane (4.50 g, 17.2 mmol, [23978-55-4]), methyl 6-{[(methanesulfonyl)oxy]methyl}pyridine-2-carboxylate (3.79 g, 15.4 mmol, [871235-14-2]) and potassium carbonate (4.74 g, 34.3 mmol) in acetonitrile (150 ml) was stirred at room temperature for 16 hours. The mixture was filtered and the filter cake was washed with acetonitrile three times. The filtrate was concentrated to give a residue. The residue was purified by silica gel column chromatography (100-200 mesh, petroleum ether/EtOAc=1:1, then 1:2, then 0:1, then EtOAc/methanol=10:1) to give to give the target compound (3.00 g, 42% yield) as yellow oil.

¹H NMR (400 MHz, DMSO-d₆): δ[ppm]=7.94-7.89 (m, 2H), 7.84 (dd, J=2.4, 6.4Hz, 1 H), 3.87 (s, 3H), 3.80 (s, 2H), 3.49-3.44 (m, 16H), 2.73 (t, J=5.6 Hz, 4H), 2.67 (t, J=4.8 Hz, 4H).

### Intermediate 8

Methyl 4-(3-tert-butoxy-3-oxo-propyl)-6-[[16-[(6-methoxycarbonyl-2-pyridyl)methyl]-1,4,10,13-tetraoxa-7,16-diazacyclooctadec-7-yl]methyl]pyridine-2-carboxylate

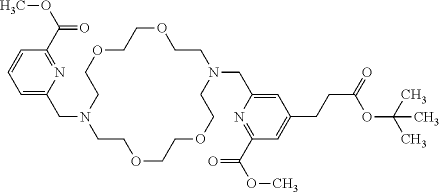 [SMILES: COC(=O)c1cccc(CN2CCOCCOCCN(Cc3cc(CCC(=O)OC(C)(C)C)cc(C(=O)OC)n3)CCOCCOCC2)n1]

A mixture of methyl 6-[(1,4,10,13-tetraoxa-7,16-diazacyclooctadecan-7-yl)methyl]pyridine-2-carboxylate (1.50 g, 3.65 mmol, Intermediate 7), methyl 4-(3-tert-butoxy-3-oxopropyI)-6-{[(methanesulfonyl)oxy]methyl}pyridine-2-carboxylate (1.09 g, 2.92 mmol, Intermediate 6), potassium carbonate (1.01 g, 7.29 mmol) and sodium iodide (50.0 mg) in acetonitrile (30 mL) was stirred at 50° C. for 16 hours. The mixture was filtered, and the filter cake was washed with acetonitrile three times. The filtrate was concentrated to give a residue. The residue was purified by reverse-phase preparative HPLC (Instrument: Agela HP1000; Column: Welch Ultimate XB_C18 150×400 mm 20/40 μm; eluent A: water/0.1% FA), eluent B: ACN; gradient: 0-30% B over 30 min; flow 100 mL/min; Detector: UV 220/254 nm) to give to give the target compound (830 mg, 33% yield) as yellow oil.

¹H NMR (400 MHz, DMSO-d₆): δ[ppm]=7.93-7.86 (m, 2H), 7.81 (dd, J=7.2, J=1.6 Hz, 1H), 7.77 (s, 1H), 7.64 (s, 1H), 3.86 (s, 3H), 3.86 (s, 3H), 3.83 (s, 2H), 3.79 (s, 2H), 3.55-3.53 (m, 8H), 3.50 (s, 8H), 2.88 (t, J=7.2 Hz, 2H), 2.76-2.74 (m, 8H), 2.57 (t, J=7.2 Hz, 2H), 1.33 (s, 9H).

### Intermediate 9 (M3)

3-[2-methoxycarbonyl-6-[[16-[(6-methoxycarbonyl-2-pyridyl)methyl]-1,4,10,13-tetraoxa-7,16-diazacyclooctadec-7-yl]methyl]-4-pyridyl]propanoic acid

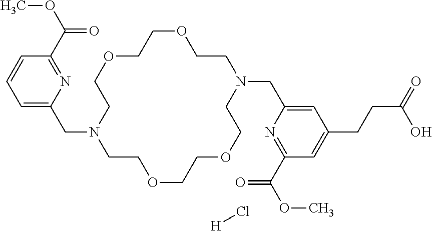

To a solution of methyl 4-(3-tert-butoxy-3-oxopropyl)-6-[(16-{[6-(methoxycarbonyl)pyridin-2-yl]methyl}-1,4,10,13-tetraoxa-7,16-diazacyclooctadecan-7-yl)methyl]pyridine-2-carboxylate (780 mg, 1.13 mmol, Intermediate 8) in 1,4-dioxane (20 mL) was added hydrochloric acid (10 mL, 4.0 M in 1,4-dioxane, 40 mmol) at 25° C. After stirring at room temperature for 16 hours, the mixture was concentrated to give a residue. The residue was dissolved in water and lyophilized to give the target compound (640 mg, 88% purity, 74% yield) as a yellow solid. Product was analyzed by analytical HPLC (gradient: 5-95% B over 0.8 min where A=water/0.0375% TFA and B=ACN//0.01875% TFA, flow rate: 1.5 mL/min, column: Chromolith@Flash RP-18E 25×2 mm, detection: UV diode array, temperature: 50° C. product retention time: 0.57 min). Further product characterization was carried out using electrospray mass spectrometry (MH+: 633.3, found m/z: 633.2).

### Intermediate 10 and 11

ethyl 3-bromo-6-(hydroxymethyl)pyridine-2-carboxylate and ethyl 5-bromo-6-(hydroxymethyl)pyridine-2-carboxylate

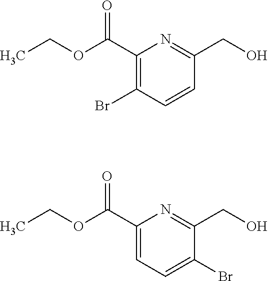

To a solution of diethyl 3-bromopyridine-2,6-dicarboxylate (50.0 g, 165 mmol, [2021236-26-8]) in ethanol (500 ml) and dichloromethane (100 ml) was addded sodium tetrahydroborate (6.26 g, 165 mmol) in portions at 0° C. After stirring at 25° C. for 12 hours, the reaction mixture was quenched by addition of saturated ammonium chloride. The resulting solution was extracted with dichloromethane. The combined organic layers were dried over anhydrous sodium sulfate, filtered and concentrated to give a residue. The residue was purified by flash silica gel column chromatography (petroleum ether: ethyl acetate=2:1) to give ethyl 3-bromo-6-(hydroxymethyl)pyridine-2-carboxylate (16 g, 37% yield, Intermediate 10) and ethyl 5-bromo-6-(hydroxymethyl)pyridine-2-carboxylate (13 g, 30% yield, Intermediate 11) as yellow oil.

### Intermediate 10

¹H NMR (400 MHz, DMSO-d₆): δ[ppm]=8.20 (d, J=8.4 Hz, 1H), 8.54 (d, J=8.4 Hz, 1H), 5.64 (t, J=6.0 Hz, 1H), 4.53 (d, J=6.0 Hz, 2H), 4.36 (q, J=7.2 Hz, 2H), 1.32 (t, J=7.2 Hz, 3H).

### Intermediate 11

¹H NMR (400 MHz, DMSO-d₆): δ[ppm]=8.25 (d, J=8.0 Hz, 1H), 7.88 (d, J=8.0 Hz, 1H), 5.38 (t, J=6.0 Hz, 1H), 4.67 (d, J=6.0 Hz, 2H), 4.35 (q, J=7.2 Hz, 2H), 1.33 (t, J=7.2 Hz, 3H).

### Intermediate 12

ethyl 3-(3-tert-butoxy-3-oxoprop-1 -en-1 -yl)-6-(hydroxymethyl)pyridine-2-carboxylate

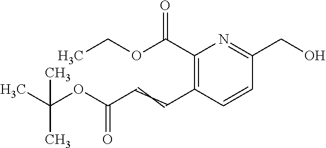 [SMILES: CCOC(=O)c1nc(CO)ccc1C=CC(=O)OC(C)(C)C]

To a solution of ethyl 3-bromo-6-(hydroxymethyl)pyridine-2-carboxylate (16.0 g, 61.5 mmol, Intermediate 10) in acetonitrile (160 ml) were added tert-butyl prop-2-enoate (11.8 g, 92.3 mmol), triethylamine (34 ml, 250 mmol), palladium(II) acetate (691 mg, 3.08 mmol) and tri-2-tolylphosphine (1.87 g, 6.15 mmol) at 25° C. After stirring at 100° C. for 16 hours under nitrogen atmosphere, the mixture was poured into water and extracted with ethyl acetate. The combined organic phase was washed with brine, dried over anhydrous sodium sulfate, filtered and concentrated to give a residue. The residue was purified by flash silica gel column chromatography (petroleum ether: ethyl acetate=1:1) to give ethyl 3-(3-tert-butoxy-3-oxoprop-1-en-1-yl)-6-(hydroxymethyl)pyridine-2-carboxylate (17.3 g, 92% yield) as yellow oil.

¹H NMR (400 MHz, DMSO-d₆): δ[ppm]=8.36 (d, J=8.0 Hz, 1H), 7.86 (d, J=16.0 Hz, 1H), 7.66 (d, J=8.0 Hz, 1H), 6.57 (d, J=16.0 Hz, 1H), 5.61 (t, J=6.0 Hz, 1H), 4.59 (d, J=6.0 Hz, 2H), 4.37 (q, J=7.2 Hz, 2H), 1.48 (s, 9H), 1.33 (t, J=7.2 Hz, 3H).

### Intermediate 13

ethyl 3-(3-tert-butoxy-3-oxopropyl)-6-(hydroxymethyl)pyridine-2-carboxylate

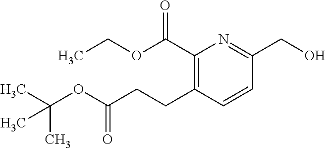 [SMILES: CCOC(=O)c1nc(CO)ccc1CCC(=O)OC(C)(C)C]

To a solution of ethyl 3-(3-tert-butoxy-3-oxoprop-1-en-1-yl)-6-(hydroxymethyppyridine-2-carboxylate (17.3 g, 56.3 mmol, Intermediate 12) in ethanol (200 ml) was added palladium on activated carbon (1.7 g, contained 50% water, 10% purity) at 20° C. After stirring at 20° C. for 16 hours under hydrogen (15 psi), the mixture was filtered through a pad of celite. The filtrate was concentrated to give ethyl 3-(3-tert-butoxy-3-oxopropyl)-6-(hydroxymethyl)pyridine-2-carboxylate product as yellow oil.

The product was combined with the material from a previous experiment (2.30 g), dissolved in ethanol and concentrated to give ethyl 3-(3-tert-butoxy-3-oxopropyl)-6-(hydroxymethyppyridine-2-carboxylate (16.5 g, 75%) as yellow oil.

LC-MS (Method 2): Rₜ=0.817 min; MS (ESIpos): m/z=310.2 [M+H]⁺.

¹H NMR (CHLOROFORM-d, 400 MHz): δ (ppm) 7.68 (d, J=8.1 Hz, 1H), 7.36 (d, J=8.1 Hz, 1H), 4.77 (s, 2H), 4.44 (q, J=7.1 Hz, 2H), 3.13 (t, J=7.6 Hz, 2H), 2.57 (t, J=7.6 Hz, 2H), 1.42 (t, J=7.1 Hz, 3H), 1.40 (s, 9H). OH is not observed.

¹³C NMR (CHLOROFORM-d, 101 MHz): δ (ppm) 171.8, 166.1, 157.4, 146.9, 140.0, 135.7, 122.7, 80.6, 64.0, 61.8, 36.4, 28.0, 27.9 (3C), 14.2.

### Intermediate 14

ethyl 5-bromo-6-({[tert-butyl(dimethyl)silyl]oxy}methyl)pyridine-2-carboxylate

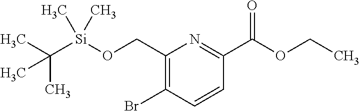 [SMILES: CCOC(=O)c1ccc(Br)c(CO[Si](C)(C)C(C)(C)C)n1]

To a mixture of ethyl 5-bromo-6-(hydroxymethyl)pyridine-2-carboxylate (13.0 g, 50.0 mmol, Intermediate 11) and imidazole (6.81 g, 100 mmol) in dichloromethane (130 ml) was added tert-butyl(chloro)dimethylsilane (9.04 g, 60.0 mmol) in portions at 0° C. After stirring at 25° C. for 16 hours, the mixture was poured into water and extracted with dichloromethane. The combined organic phase was washed with brine, dried over anhydrous sodium sulfate, filtered and concentrated to give a residue. The residue was purified by flash silica gel column chromatography (petroleum ether: ethyl acetate=20:1) to give ethyl 5-bromo-6-({[tert-butyl(dimethypsilyl]oxy}methyppyridine-2-carboxylate (18.0 g, 96% yield) as yellow oil.

¹H NMR (400 MHz, DMSO-d₆): δ[ppm]=8.25 (d, J=8.4 Hz, 1H), 7.88 (d, J=8.4 Hz, 1H), 4.87 (s, 2H), 4.34 (q, J=7.2 Hz, 2H), 1.32 (t, J=7.2 Hz, 3H), 0.87 (s, 9H), 0.09 (s, 6H).

### Intermediate 15

ethyl 5-(3-tert-butoxy-3-oxoprop-1-en-1-yl)-6-({[tert-butyl(dimethyl)silyl]oxy}methyl)pyridine-2-carboxylate

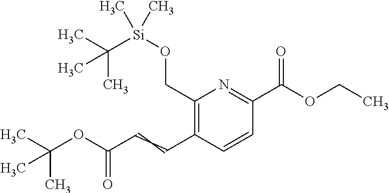 [SMILES: CCOC(=O)c1ccc(C=CC(=O)OC(C)(C)C)c(CO[Si](C)(C)C(C)(C)C)n1]

To a solution of ethyl 5-bromo-6-({[tert-butyl(dimethypsilyl]oxy}methyl)pyridine-2-carboxylate (18.0 g, 48.1 mmol, Intermediate 14) in acetonitrile (200 ml) was added tert-butyl prop-2-enoate (9.24 g, 72.1 mmol), triethylamine (27 ml, 190 mmol), palladium(II) acetate (540 mg, 2.40 mmol) and tri-2-tolylphosphine (1.46 g, 4.81 mmol) at 25° C. After stirring at 100° C. for 16 hours under nitrogen atmosphere, the mixture was poured into water and extracted with ethyl acetate. The combined organic phase was washed with brine, dried over anhydrous sodium sulfate, filtered and concentrated to give a residue. The residue was purified by flash silica gel column chromatography (petroleum ether: ethyl acetate=20:1) to give ethyl 5-(3-tert-butoxy-3-oxoprop-1-en-1-yl)-6-({[tert-butyl(dimethypsilyl]oxy}methyl)pyridine-2-carboxylate (19.0 g, 94% yield) as yellow oil.

¹H NMR (400 MHz, DMSO-d₆): δ[ppm]=8.37 (d, J=8.4 Hz, 1H), 7.99 (d, J=8.4 Hz, 1H), 7.90 (d, J=16.0 Hz, 1H), 6.62 (d, J=16.0 Hz, 1H), 4.91 (s, 2H), 4.35 (q, J=6.8 Hz, 2H), 1.48 (s, 9H), 1.33 (t, J=6.8 Hz, 3H), 0.83 (s, 9H), 0.08 (5, 6H).

### Intermediate 16

ethyl 5-(3-tert-butoxy-3-oxopropyl)-6-({[tert-butyl(dimethyl)silyl]oxy}methyl)pyridine-2-carboxylate

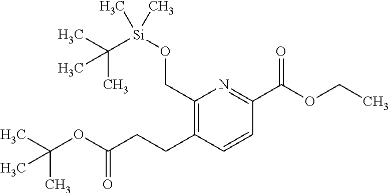 [SMILES: CCOC(=O)c1ccc(CCC(=O)OC(C)(C)C)c(CO[Si](C)(C)C(C)(C)C)n1]

To a solution of ethyl 5-(3-tert-butoxy-3-oxoprop-1-en-1-yl)-6-({[tert-butyl(dimethypsilyl]-oxy}methyl)pyridine-2-carboxylate (19.0 g, 45.1 mmol, Intermediate 15) in ethanol (200 ml) was added palladium on activated carbon (1.77 g, contained 50% water, 10% purity) at 20° C. After stirring at 50° C. for 16 hours under hydrogen (15 psi), the mixture was filtered through a pad of celite. The filtrate was concentrated to give ethyl 5-(3-tert-butoxy-3-oxopropyl)-6-({[tert-butyl(dimethypsilyl]oxy}methyppyridine-2-carboxylate (19.0 g, 99% yield) as yellow oil.

¹H NMR (400 MHz, DMSO-d₆): δ[ppm]=7.92 (d, J=8.4 Hz, 1H), 7.83 (d, J=8.0 Hz, 1H), 4.84 (s, 2H), 4.33 (q, J=6.8 Hz, 2H), 3.00 (t, J=8.0 Hz, 2H), 2.60 (t, J=8.0 Hz, 2H), 1.37 (s, 9H), 1.32 (t, J=6.8 Hz, 3H), 0.86 (s, 9H), 0.08 (s, 6H).

### Intermediate 17

ethyl 5-(3-tert-butoxy-3-oxopropyI)-6-(hydroxymethyl)pyridine-2-carboxylate

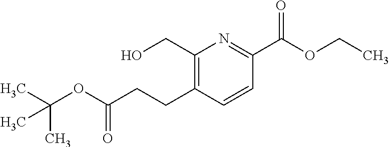 [SMILES: CCOC(=O)c1ccc(CCC(=O)OC(C)(C)C)c(CO)n1]

To a mixture of ethyl 5-(3-tert-butoxy-3-oxopropyl)-6-({[tert-butyl(dimethypsilyl]oxy}methyl)-pyridine-2-carboxylate (19.0 g, 44.9 mmol, Intermediate 16) in tetrahydrofuran (200 ml) was added tetra-N-butylammonium fluoride (54 ml, 1.0 M in tetrahydrofuran, 54 mmol) at room temperature. After stirring at room temperature for 0.5 hour, the mixture was concentrated. The residue was combined with the material from an earlier experiment (4.30 g), dissolved in water and extracted with ethyl acetate. The combined organic phase was washed with brine, dried over anhydrous sodium sulfate, filtered and concentrated to give a residue. The residue was purified by flash column chromatography (petroleum ether: ethyl acetate=3: 2) to give ethyl 5-(3-tert-butoxy-3-oxopropyl)-6-(hydroxmethyppyridine-2-carboxylate (13.5 g, 74%) as yellow oil.

LC-MS (Method 2): Rₜ=0.867 min; MS (ESIpos): m/z=310.2 [M+H]⁺.

¹H NMR (DMSO-d₆, 600 MHz): δ (ppm) 7.92 (d, J=8.0 Hz, 1H, H-3), 7.82 (d, J=8.0 Hz, 1H, H-4), 5.31 (t, J=5.7 Hz, 1H, OH), 4.66 (d, J=5.7 Hz, 2H, 6-CH₂), 4.34 (q, J=7.0 Hz, 2H, 2-OCH₂), 3.00 (t, J=7.6 Hz, 2H, 5-CH₂), 2.61 (t, J=7.6 Hz, 2H, 5-CH₂CO), 1.37 (s, 9H, t-Bu), 1.33 (t, J=7.1 Hz, 3H, 2-CH₃). The assignment given is consistent with NOESY and COSY experiments.

¹³C NMR (CHLOROFORM-d, 101 MHz): δ (ppm) 171.2, 164.9, 156.5, 144.6, 137.1, 136.6, 123.8, 81.2, 61.7, 61.5, 34.4, 28.0 (3C), 25.3, 14.3.

### EXPERIMENTAL SECTION—EXAMPLES

### Dimeric Chelators

### Example 1 (Dim1)

Dimethyl 4,4′-{[9,13-bis(2-aminoethyl)-1,5,17,21-tetraoxo-3,19-dioxa-6,9,13,16-tetraazahenicosane-1,21-diyl]diimino}bis{6-[(16-{[6-(methoxycarbonyl)pyridin-2-yl]methyl}-1,4,10,13-tetraoxa-7,16-diazacyclooctadecan-7-yl)methyl]pyridine-2-carboxylate}

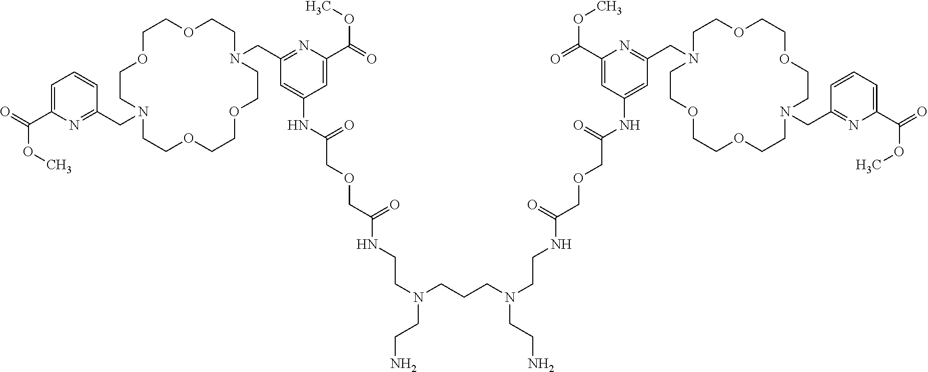 [SMILES: COC(=O)c1cccc(CN2CCOCCOCCN(Cc3cc(NC(=O)COCC(=O)NCCN(CCN)CCCN(CCN)CCNC(=O)COCC(=O)Nc4cc(CN5CCOCCOCCN(Cc6cccc(C(=O)OC)n6)CCOCCOCC5)nc(C(=O)OC)c4)cc(C(=O)OC)n3)CCOCCOCC2)n1]

N,N,N′,N′-tetrakis(2-aminoethyl)propane-1,3-diamine (4.5 mg, [871235-14-2]), [2-({2-(methoxycarbonyl)-6-[(16-{[6-(methoxycarbonyl)pyridin-2-yl]nethyl}-1,4,10,13-tetraoxa-7,16-diazacyclooctadecan-7-yl)methyl]pyridin-4-yl}amino)-2-oxoethoxy]acetic acid (13.3 mg, Intermediate 3) and PyAOP (10 mg) were dissolved in NMP (1 mL). DIPEA (11.2 μL) was added and reaction left for 24 hrs. Reaction mixture was diluted with water/0.1% TFA (8 mL) and product purified by preparative HPLC (column: Phenomenex Luna 5 μm C18(2) 100 Å, 250×50 mm; gradient: 10-40% B over 40 min where A=water/0.1% TFA and B=ACN; flow: 10 mL/min; detection: UV 214/254 nm) affording 7.6 mg (74% yield) of the target compound after freeze-drying. Purified product was analyzed by analytical HPLC (gradient: 10-40% B over 2.5 min where A=water/0.1% TFA and B=ACN, flow rate: 0.5 mL/min, column: Waters Acquity BEH C18, 1.7 μm, 2.1×50 mm, detection: UV diode array, product retention time: 1.43 min). Further product characterization was carried out using electrospray mass spectrometry (MH⁺: 1593.8, found m/z: 1593.9).

### Example 2 (Dim2)

6,6′-[pyridine-2,6-diylbis(methylene-1,4,10,13-tetraoxa-7,16-diazacyclooctadecane-16,7-diylmethylene)]dipyridine-2-carboxylic acid

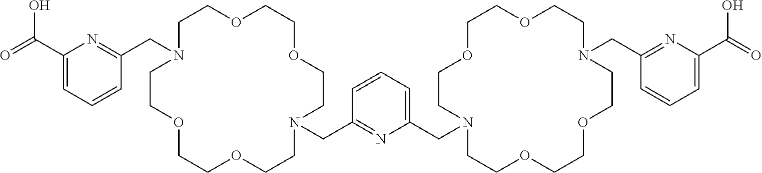 [SMILES: O=C(O)c1cccc(CN2CCOCCOCCN(Cc3cccc(CN4CCOCCOCCN(Cc5cccc(C(=O)O)n5)CCOCCOCC4)n3)CCOCCOCC2)n1]

6-(1,4,10,13-tetraoxa-7,16-diazacyclooctadec-7-ylmethyl)pyridine-2-carboxylic acid (238 mg, 0.507 mmol, prepared as described in Angewandte Chemie, Nikki et al, 2017) was mixed with Na₂CO₃ (70 mg, 0.660 mmol) in ACN (10 mL). DIPEA (0.44 mL, 2.538 mmol) was added. The solution was heated to reflux and stirred for 10 min, then 2,6-bis-(bromomethyl)pyridine (40 mg, 0.152 mmol) in ACN (5 mL) was added and stirred for 3 days under nitrogen. The solution was filtered and evaporated in vacuo. Residue was diluted with water/0.1% TFA (8 mL) and product purified by preparative HPLC (column: Phenomenex Luna 5 μm C18(2) 100 Å, 250×50 mm; gradient: 5-30% B over 40 min where A=water/0.1% TFA and B=ACN; flow: 10 mL/min; detection: UV 214/254 nm) affording 36.7 mg (27% yield) of the target compound after freeze-drying. Purified product was analyzed by analytical HPLC (gradient: 5-30% B over 2.5 min where A=water/0.1% TFA and B=ACN, flow rate: 0.5 mL/min, column: Waters Acquity BEH C18, 1.7 μm, 2.1×50 mm, detection: UV diode array, product retention time: 1.42 min). Further product characterization was carried out using electrospray mass spectrometry (MH⁺: 898.5, found m/z: 898.5).

### Example 3 (Dim3)

6-[[16-[[6-[[(5R)-5-carboxy-5-[[6-[[16-[(6-carboxy-2-pyridyl)methyl]-1,4,10,13-tetraoxa-7,16-diazacyclooctadec-7-yl]methyl]pyridine-2-carbonyl]amino]pentyl]carbamoyl]-2-pyridyl]methyl]-1,4,10,13-tetraoxa-7,16-diazacyclooctadec-7-yl]methyl]pyridine-2-carboxylic acid

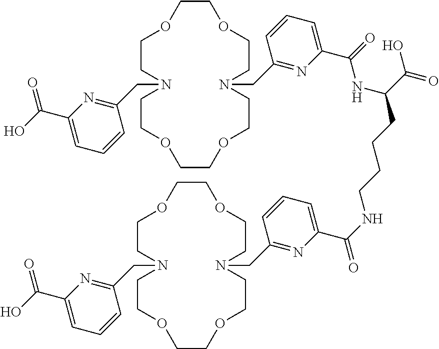 [SMILES: O=C(O)c1cccc(CN2CCOCCOCCN(Cc3cccc(C(=O)NCCCC[C@@H](NC(=O)c4cccc(CN5CCOCCOCCN(Cc6cccc(C(=O)O)n6)CCOCCOCC5)n4)C(=O)O)n3)CCOCCOCC2)n1]

D-lysine (0.5 mg) and bis(2,3,5,6-tetrafluorophenyl) 6,6′-[1,4,10,13-tetraoxa-7,16-diazacyclooctadecane-7,16-diyIbis(methylene)]dipyridine-2-carboxylate (Example 15; 5.7 mg) were dissolved in PBS (1 mL) and NMP (0.4 mL) and solution heated at 40-60° C. for 5 hours. Solution was diluted with water/0.1% TFA (8 mL) and product isolated by preparative HPLC purification (column: Phenomenex Luna 5 μm C18(2) 100 Å, 250×50 mm; gradient: 5-30% B over 40 min where A=water/0.1% TFA and B=ACN; flow: 10 mL/min; detection: UV214/254 nm) affording 7.6 mg (74% yield) of the target compound after freeze-drying. Purified product was analyzed by analytical HPLC (gradient: 10-50% B over 2.5 min where A=water/0.1% TFA and B=ACN, flow rate: 0.5 mL/min, column: Waters Acquity BEH C18, 1.7 μm, 2.1×50 mm, detection: UV diode array, product retention time: 1.02 min). Further product characterization was carried out using electrospray mass spectrometry (MH⁺: 1175.6, found m/z: 1175.6).

### Trimeric Chelators

### Example 4 (Tri1)

Dimethyl 4,4′-{[13-(2-aminoethyl)-9-(2-{2-[2-({2-(methoxycarbonyl)-6-[(16-{[6-(methoxycarbonyl)pyridin-2-yl]methyl}-1,4,10,13-tetraoxa-7,16-diazacyclooctadecan-7-yl)methyl]pyridin-4-yl}amino)-2-oxoethoxy]acetamido}ethyl)-1,5,17,21-tetraoxo-3,19-dioxa-6,9,13,16-tetraazahenicosane-1,21-diyl]diimino}bis{6-[(16-{[6-(methoxycarbonyl)pyridin-2-yl]methyl}-1,4,10,13-tetraoxa-7,16-diazacyclooctadecan-7-yl)methyl]pyridine-2-carboxylate}

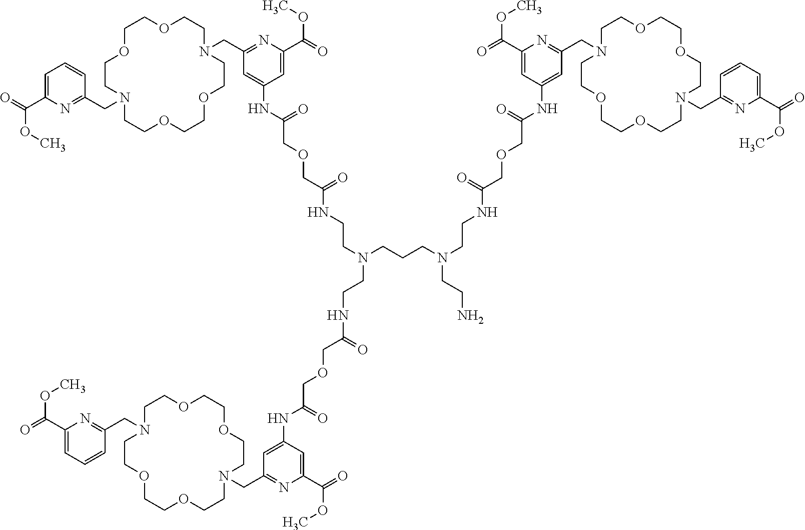 [SMILES: COC(=O)c1cccc(CN2CCOCCOCCN(Cc3cc(NC(=O)COCC(=O)NCCN(CCN)CCCN(CCNC(=O)COCC(=O)Nc4cc(CN5CCOCCOCCN(Cc6cccc(C(=O)OC)n6)CCOCCOCC5)nc(C(=O)OC)c4)CCNC(=O)COCC(=O)Nc4cc(CN5CCOCCOCCN(Cc6cccc(C(=O)OC)n6)CCOCCOCC5)nc(C(=O)OC)c4)cc(C(=O)OC)n3)CCOCCOCC2)n1]

N,N,N′,N′-tetrakis(2-aminoethyl)propane-1,3-diamine (8 mg, [871235-14-2]), [2-({2-(methoxycarbonyl)-6-[(16-{[6-(methoxycarbonyl)pyridin-2-yl]nethyl}-1,4,10,13-tetraoxa-7,16-diazacyclooctadecan-7-yl)methyl]pyridin-4-yl}amino)-2-oxoethoxy]acetic acid (23.6 mg, Intermediate 3) and PyAOP (17.8 mg) were dissolved in NMP (1 mL). DIPEA (23.8 μL) was added and reaction left for 24 hours. Reaction mixture was diluted with water/0.1% TFA (8 mL) and product purified by preparative HPLC (column: Phenomenex Luna 5 μm C18(2) 100 Å, 250×50 mm; gradient: 10-40% B over 40 min where A=water/0.1% TFA and B=ACN; flow: 10 mL/min; detection: UV 214/254 nm) affording 8.8 mg (34% yield) of the target compound after freeze-drying. Purified product was analyzed by analytical HPLC (gradient: 10-50% B over 2.5 min where A=water/0.1% TFA and B=ACN, flow rate: 0.5 mL/min, column: Waters Acquity BEH C18, 1.7 μm, 2.1×50 mm, detection: UV diode array, product retention time: 1.30 min). Further product characterization was carried out using electrospray mass spectrometry (MH₂²⁺: 1134.1, found m/z: 1134.1).

### Example 5

2-[2-[2-[3-[bis[2-[[2-[2-[[2-methoxycarbonyl-6-[[16-[(6-methoxycarbonyl-2-pyridyl)methyl]-1,4,10,13-tetraoxa-7,16-diazacyclooctadec-7-yl]methyl]-4-pyridyl]amino]-2-oxo-ethoxy]acetyl]amino]ethyl]amino]propyl-[2-[[2-[2-[[2-methoxycarbonyl-6-[[16-[(6-methoxycarbonyl-2-pyridyl)methyl]-1,4,10,13-tetraoxa-7,16-diazacycloociadec-7-yl]methyl]-4-pyridyl]amino]-2-oxo-ethoxy]acetyl]amino]ethyl]amino]ethylamino]-2-oxo-

[symbol]

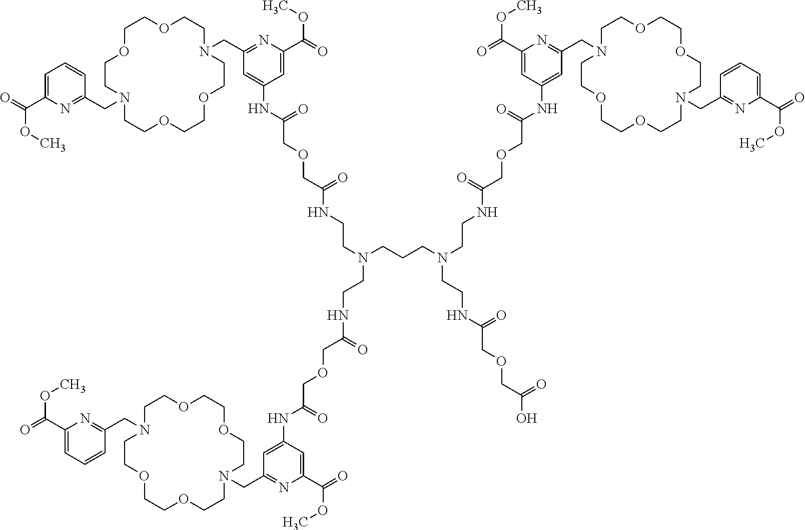 [SMILES: COC(=O)c1cccc(CN2CCOCCOCCN(Cc3cc(NC(=O)COCC(=O)NCCN(CCCN(CCNC(=O)COCC(=O)Nc4cc(CN5CCOCCOCCN(Cc6cccc(C(=O)OC)n6)CCOCCOCC5)nc(C(=O)OC)c4)CCNC(=O)COCC(=O)Nc4cc(CN5CCOCCOCCN(Cc6cccc(C(=O)OC)n6)CCOCCOCC5)nc(C(=O)OC)c4)CCNC(=O)COCC(=O)O)cc(C(=O)OC)n3)CCOCCOCC2)n1]

dimethyl 4,4′-{[13-(2-aminoethyl)-9-(2-{2-[2-({2-(methoxycarbonyl)-6-[(16-{[6-(methoxycarbonyl)pyridin-2-yl]methyl}-1,4,10,13-tetraoxa-7,16-diazacyclooctadecan-7-yl)methyl]pyridin-4-yl}amino)-2-oxoethoxy]acetamido}ethyl)-1,5,17,21-tetraoxo-3,19-dioxa-6,9,13,16-tetraazahenicosane-1,21-diyl]diimino}bis{6-[(16-{[6-(methoxycarbonyl)pyridin-2-yl]methyl}-1,4,10,13-tetraoxa-7,16-diazacyclooctadecan-7-yl)methyl]pyridine-2-carboxylate} (11.4 mg, example 4), diglycolic anhydride (2.9 mg) and DPEA (4.4 μL) were dissolved in NMP (1 mL) and solution left for 24 hours. Solution was diluted with water/0.1% TFA (8 mL) and product purified by preparative HPLC (column: Phenomenex Luna 5 μm C18(2) 100 Å, 250×50 mm; gradient: 10-50% B over 40 min where A=water/0.1% TFA and B=ACN; flow: 10 mL/min; detection: UV 214/254 nm) to afford 6.2 mg (52% yield) of the target compound after freeze-drying. Purified product was analyzed by analytical HPLC (gradient: 10-50% B over 2.5 min where A=water/0.1% TFAand B=ACN, flow rate: 0.5 mL/min, column: Waters Acquity BEH C18, 1.7 μm, 2.1×50 mm, detection: UV diode array, product retention time: 1.31 min). Further product characterization was carried out using electrospray mass spectrometry (MH⁺: 2383.3, found m/z: 2383.2).

### Tetrameric Chelators

### Example 6 (Tet1)

Dimethyl 4,4′-{[9,13-bis(2-{2-[2-({2-(methoxycarbonyl)-6-[(16{[6-(methoxycarbonyl)pyridin-2-yl]methyl}-1,4,10,13-tetraoxa-7,16-diazacyclooctadecan-7-yl)methyl]pyridin-4-yl}amino)-2-oxoethoxy]acetamido}ethyl)-1,5,17,21-tetraoxo-3,19-dioxa-6,9,13,16-tetraazahenicosane-1,21-diyl]diimino}bis{6-[(16-{[6-(methoxycarbonyl)pyridin-2-yl]methyl}-1,4,10,13-tetraoxa-7,16-diazacyclooctadecan-7-yl)methyl]pyridine-2-carboxylate}

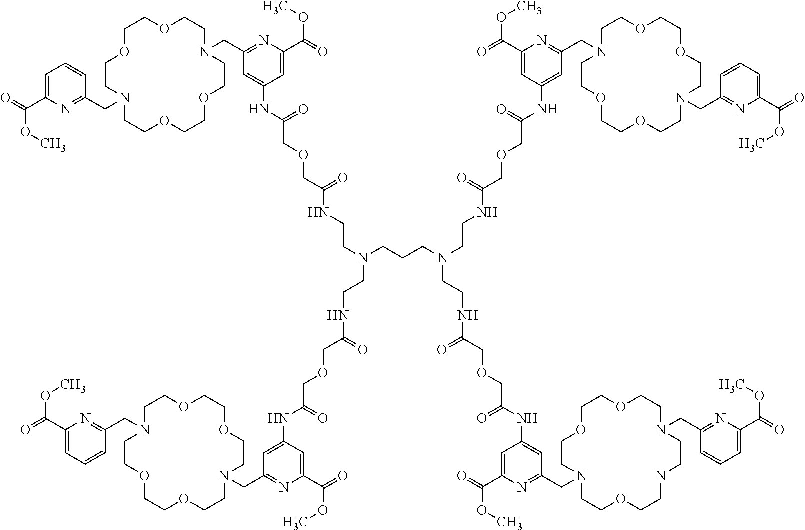 [SMILES: COC(=O)c1cccc(CN2CCNCCOCCN(Cc3cc(NC(=O)COCC(=O)NCCN(CCCN(CCNC(=O)COCC(=O)Nc4cc(CN5CCOCCOCCN(Cc6cccc(C(=O)OC)n6)CCOCCOCC5)nc(C(=O)OC)c4)CCNC(=O)COCC(=O)Nc4cc(CN5CCOCCOCCN(Cc6cccc(C(=O)OC)n6)CCOCCOCC5)nc(C(=O)OC)c4)CCNC(=O)COCC(=O)Nc4cc(CN5CCOCCOCCN(Cc6cccc(C(=O)OC)n6)CCOCCOCC5)nc(C(=O)OC)c4)cc(C(=O)OC)n3)CCOCCOCC2)n1]

N,N,N′,N′-tetrakis(2-aminoethyl)propane-1,3-diamine (2 mg, [871235-14-2]), [2-({2-(methoxycarbonyl)-6-[(16-{[6-(methoxycarbonyl)pyridin-2-yl]nethyl}-1,4,10,13-tetraoxa-7,16-diazacyclooctadecan-7-yl)methyl]pyridin-4-yl}amino)-2-oxoethoxy]acetic acid (16.7 mg, intermediate 3) and PyAOP (7.4 mg) were dissolved in NMP (1 mL). DI PEA (9.9 μL) was added and reaction left for 1 hour. Reaction mixture was diluted with water/0.1% TFA (8 mL) and the product purified by preparative HPLC (column: Phenomenex Luna 5 μm C18(2) 100 Å, 250×50 mm; gradient: 10-50% B over 40 min where A=water/0.1% TFA and B=ACN; flow: 10 mL/min; detection: UV 214/254 nm) affording 8 mg (96% yield) of the target compound after freeze-drying. Purified product was analyzed by analytical HPLC (gradient: 10-50% B over 2.5 min where A=water/0.1% TFA and B=ACN, flow rate: 0.5 mL/min, column: Waters Acquity BEH C18, 1.7 μm, 2.1×50 mm, detection: UV diode array, product retention time: 1.47 min). Further product characterization was carried out using electrospray mass spectrometry (MH⁺: 2940.4, found m/z: 2940.4).

### Example 7 (Tet2)

4,4′-[(9,13-bis{2-[2-(2-{[2-carboxy-6-({16-[(6-carboxypyridin-2-yl)methyl]-1,4,10,13-tetraoxa-7,16-diazacyclooctadecan-7-yl}methyl)pyridin-4-yl]amino}-2-oxoethoxy)acetamido]ethyl}-1,5,17,21-tetraoxo-3,19-dioxa-6,9,13,16-tetraazahenicosane-1,21-diyl)diimino]bis[6-({16-[(6-carboxypyridin-2-yl)methyl]-1,4,10,13-tetraoxa-7,16-diazacyclooctadecan-7-yl}methyl)pyridine-2-carboxylic acid]

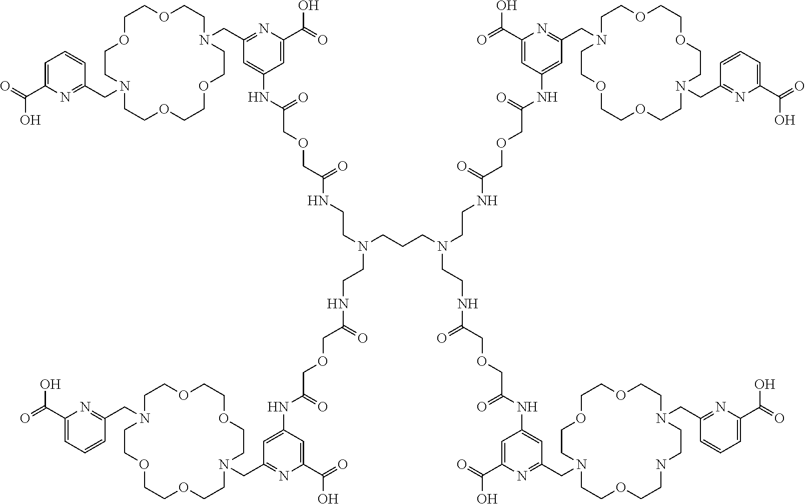 [SMILES: O=C(COCC(=O)Nc1cc(CN2CCOCCNCCN(Cc3cccc(C(=O)O)n3)CCOCCOCC2)nc(C(=O)O)c1)NCCN(CCCN(CCNC(=O)COCC(=O)Nc1cc(CN2CCOCCOCCN(Cc3cccc(C(=O)O)n3)CCOCCOCC2)nc(C(=O)O)c1)CCNC(=O)COCC(=O)Nc1cc(CN2CCOCCOCCN(Cc3cccc(C(=O)O)n3)CCOCCOCC2)nc(C(=O)O)c1)CCNC(=O)COCC(=O)Nc1cc(CN2CCOCCOCCN(Cc3cccc(C(=O)O)n3)CCOCCOCC2)nc(C(=O)O)c1]

Dimethyl 4,4′-{[9,13-bis(2-{2-[2-({2-(methoxycarbonyl)-6-[(16-{[6-(methoxycarbonyl)pyridin-2-yl]nethyl}-1,4,10,13-tetraoxa-7,16-diazacyclooctadecan-7-yl)methyl]pyridin-4-yl}amino)-2-oxoethoxy]acetamido}ethyl)-1,5,17,21-tetraoxo-3,19-dioxa-6,9,13,16-tetraazahenicosane-1,21-diyl]diimino}bis{6-[(16-{[6-(methoxycarbonyl)pyridin-2-yl]methyl}-1,4, 10,13-tetraoxa-7,16-diazacyclooctadecan-7-yl)methyl]pyridine-2-carboxylate} (2.7 mg, Example 6) was dissolved in 2.5% ammonia/10% ACN (1 mL) and solution left for one day. Solution was diluted with water/0.1% TFA (8 mL), adjusted to pH 2 with TFA (20 μL) and the product purified by preparative HPLC (column: Phenomenex Luna 5 μm C18(2) 100 Å, 250×50 mm; gradient: 10-50% B over 40 min where A=water/0.1% TFA and B=ACN; flow: 10 mL/min; detection: UV 214/254 nm) affording 1.7 mg (65% yield) of the target compound after freeze-drying. Purified product was analyzed by analytical HPLC (gradient: 10-50% B over 2.5 min where A=water/0.1% TFA and B=ACN, flow rate: 0.5 mL/min, column: Waters Acquity BEH C18, 1.7 μm, 2.1×50 mm, detection: UV diode array, product retention time: 1.9 min). Further product characterization was carried out using electrospray mass (MH₃³⁺: 943.8, found m/z: 943.8).

### Example 8 (Tet3)

Dimethyl 4,4′-{[8,8-bis({2-[2-({2-(methoxycarbonyl)-6-[(16-[6-(methoxycarbonyl)pyridin-2-yl]methyl)-1,4,10,13-tetraoxa-7,16-diazacyclooctadecan-7-yl)methyl]pyridin-4-yl}amino)-2-oxoethoxy]acetamido}methyl)-1,5,11,15-tetraoxo-3,13-dioxa-6,10-diazapentadecane-1,15-diyl]diimino}bis{6-[(16-{[6-(methoxycarbonyl)pyridin-2-yl]methyl}-1,4,10,13-tetraoxa-7,16-diazacyclooctadecan-7-yl)methyl]pyridine-2-carboxylate}

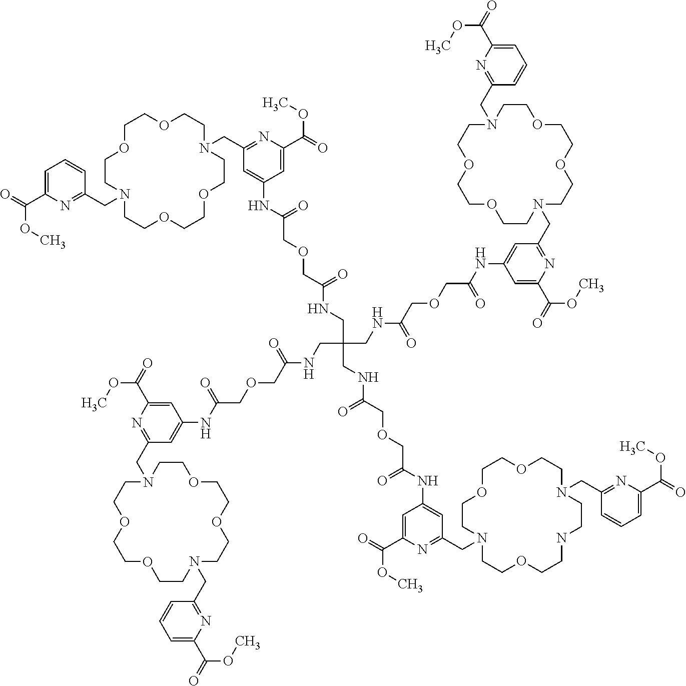 [SMILES: COC(=O)c1cccc(CN2CCNCCOCCN(Cc3cc(NC(=O)COCC(=O)NCC(CNC(=O)COCC(=O)Nc4cc(CN5CCOCCOCCN(Cc6cccc(C(=O)OC)n6)CCOCCOCC5)nc(C(=O)OC)c4)(CNC(=O)COCC(=O)Nc4cc(CN5CCOCCOCCN(Cc6cccc(C(=O)OC)n6)CCOCCOCC5)nc(C(=O)OC)c4)CNC(=O)COCC(=O)Nc4cc(CN5CCOCCOCCN(Cc6cccc(C(=O)OC)n6)CCOCCOCC5)nc(C(=O)OC)c4)cc(C(=O)OC)n3)CCOCCOCC2)n1]

2,2-bis(aminomethyl)propane-1,3-diamine tetrahydrochloride (1 mg, [14302-75-1]), [2-({2-(methoxycarbonyl)-6-[(16-{[6-(methoxycarbonyl)pyridin-2-yl]methyl}-1,4,10,13-tetraoxa-7,16-diazacyclooctadecan-7-yl)methyl]pyridin-4-yl}amino)-2-oxoethoxy]acetic acid (14.2 mg, Intermediate 3) and PyAOP (25.3 mg) were dissolved in NMP (1 mL). DIPEA (18.8 μL) was added and reaction heated at 60° C. for 2 days. Reaction mixture was diluted with water/0.1% TFA (8 mL) and the product purified by preparative HPLC (column: Phenomenex Luna 5 μm C18(2) 100 Å, 250×50 mm; gradient: 10-50% B over 40 min where A=water/0.1% TFA and B=ACN; flow: 10 mL/min; detection: UV 214/254 nm) affording 6.6 mg (65% yield) of the target compound after freeze-drying. Purified product was analyzed by analytical HPLC (gradient: 10-50% B over 2.5 min where A=water/0.1% TFA and B=ACN, flow rate: 0.5 mL/min, column: Waters Acquity BEH C18, 1.7 μm, 2.1×50 mm, detection: UV diode array, product retention time: 1.53 min). Further product characterization was carried out using electrospray mass spectrometry (MH₂²⁺: 1413.7, found m/z: 1413.7).

### Example 9 (Tet4)

Dimethyl 4,4′-{7,11-bis[2-(3-{2-(methoxycarbonyl)-6-[(16-{[6-(methoxycarbonyl)pyridin-2-yl]methyl}-1,4,10,13-tetraoxa-7,16-diazacyclooctadecan-7-yl)methylipyridin-4-yl}propanamido)ethyl]-3,15-dioxo-4,7,11,14-tetraazaheptadecane-1,17-diyl}bis{6-[(16-{[6-(methoxycarbonyl)pyridin-2-yl]methyl}-1,4,10,13-tetraoxa-7,16-diazacyclooctadecan-7-yl]methyl]pyridine-2-carboxylate}

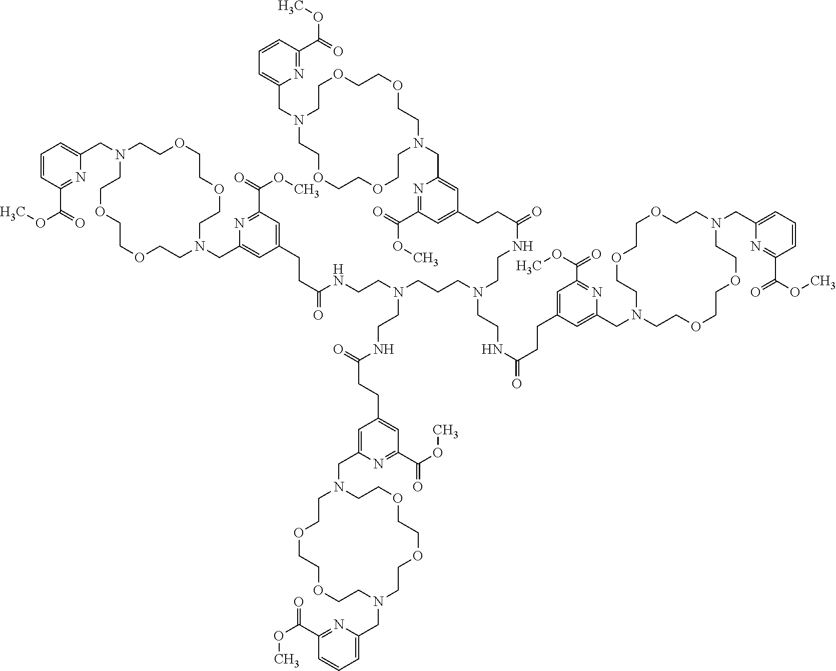

N,N,N′,N′-tetrakis(2-aminoethyl)propane-1,3-diamine (15 mg, [871235-14-2]), 3-[2-methoxycarbonyl-6-[[16-[(6-methoxycarbonyl-2-pyridyl) methyl]-1,4,10,13-tetraoxa-7,16-diazacyclooctadec-7-yl]methyl]-4-pyridyl]propanoic acid (81 mg, Intermediate 9) and PyAOP (94.6 mg) were dissolved in NMP (1 mL). DIPEA (149 μL) was added and reaction left for 20 min. Two more portions (20 mg and 8 mg) of PyAOP were added and reaction left for 20 min after each addition. Reaction mixture was diluted with water/0.1% TFA (8 mL) and the product purified by preparative HPLC (column: Phenomenex Luna 5 μm C18(2) 100 Å, 250×50 mm; gradient: 10-40% B over 40 min where A=water/0.1% TFA and B=ACN; flow: 10 mL/min; detection: UV 214/254 nm) affording 47 mg (81% yield) of the target compound after freeze-drying. Purified product was analyzed by analytical HPLC (gradient: 10-40% B over 2.5 min where A=water/0.1% TFA and B=ACN, flow rate: 0.5 mL/min, column: Waters Acquity BEH C18, 1.7 μm, 2.1×50 mm, detection: UV diode array, product retention time: 1.69 min). Further product characterization was carried out using electrospray mass spectrometry (MH⁺: 2704.4, found m/z: 2704.5).

### Example 10 (Tet5)

4,4′-[7,11-bis(2-{3-[2-carboxy-6-({16-[(6-carboxypyridin-2-yl)methyl]-1,4,10,13-tetraoxa-7,16-diazacyclooctadecan-7-yl}methyl)pyridin-4-yl]propanamido}ethyl)-3,15-dioxo-4,7,11,14-tetraazaheptadecane-1,17-diyl]bis[6-({16-[(6-carboxypyridin-2-yl)methyl]-1,4,10,13-tetraoxa-7,16-diazacyclooctadecan-7-yl}methyl)pyridine-2-carboxylic acid]

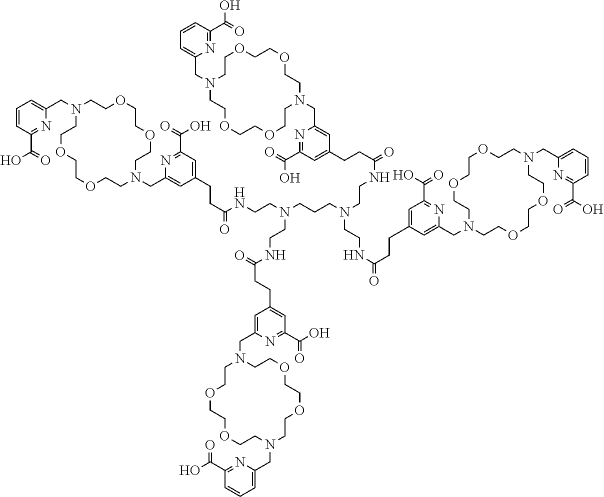 [SMILES: O=C(CCc1cc(CN2CCOCCOCCN(Cc3cccc(C(=O)O)n3)CCOCCOCC2)nc(C(=O)O)c1)NCCN(CCCN(CCNC(=O)CCc1cc(CN2CCOCCOCCN(Cc3cccc(C(=O)O)n3)CCOCCOCC2)nc(C(=O)O)c1)CCNC(=O)CCc1cc(CN2CCOCCOCCN(Cc3cccc(C(=O)O)n3)CCOCCOCC2)nc(C(=O)O)c1)CCNC(=O)CCc1cc(CN2CCOCCOCCN(Cc3cccc(C(=O)O)n3)CCOCCOCC2)nc(C(=O)O)c1]

Dimethyl 4,4′-{7,11-bis[2-(3-{2-(methoxycarbonyl)-6-[(16-{[6-(methoxycarbonyl)pyridin-2-yl]methyl}-1,4,10,13-tetraoxa-7,16-diazacyclooctadecan-7-yl)methyl]pyridin-4-yl}propanamido)ethyl]-3,15-dioxo-4,7,11,14-tetraazaheptadecane-1,17-diyl}bis{6-[(16-{[6-(methoxycarbonyl)pyridin-2-yl]methyl}-1,4,10,13-tetraoxa-7,16-diazacyclooctadecan-7-yl)methyl]pyridine-2-carboxylate} (47 mg, Example 9) was dissolved in 20% ACN/water (2 mL). 5 M NaOH (100 μL) was added and solution left for 1 hour, then adjusted to pH 2 with TFA (50 μL), diluted with water/0.1% TFA (7 mL) and product purified by preparative HPLC (column: Phenomenex Luna 5 μm C18(2) 100 Å, 250×50 mm; gradient: 10-40% B over 40 min where A=water/0.1% TFA and B=ACN; flow: 10 mL/min; detection: UV 214/254 nm) affording 33 mg (70% yield) of the target compound after freeze-drying. Purified product was analyzed by analytical HPLC (gradient: 10-40% B over 2.5 min where A=water/0.1% TFA and B=ACN, flow rate: 0.5 mL/min, column: Waters Acquity BEH C18, 1.7 μm, 2.1×50 mm, detection: UV diode array, product retention time: 1.03 min). Further product characterization was carried out using electrospray mass spectrometry (MH⁺: 2592.3, found m/z: 2592.4).

### Example 11 (Tet6)

5,5′-[7,11-bis(2-{3-[6-carboxy-2-({16-[(6-carboxypyridin-2-yl)methyl]-1,4,10,13-tetraoxa-7,16-diazacyclooctadecan-7-yl}methyl)pyridin-3-yl]propanamido}ethyl)-3,15-dioxo-4,7,11,14-tetraazaheptadecane-1,17-diyl]bis(6-{[16-(3-carboxybenzyl)-1,4,10,13-tetraoxa-7,16-diazacyclooctadecan-7-yl]methyl}pyridine-2-carboxylic acid)

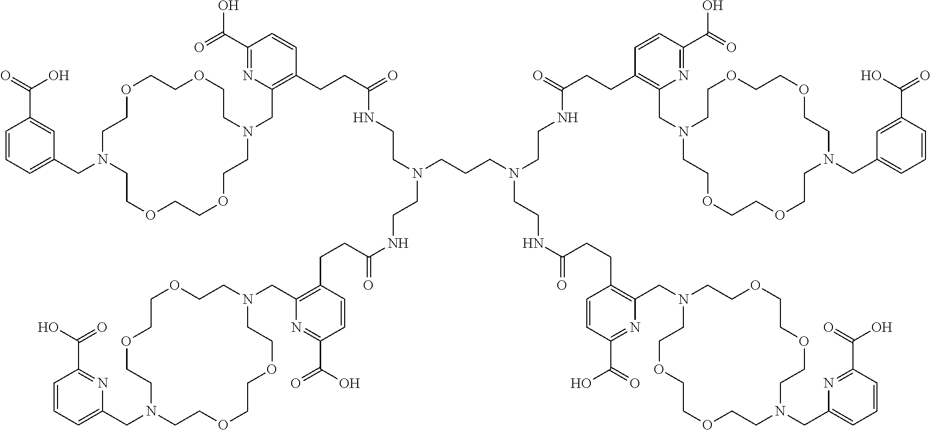 [SMILES: O=C(CCc1ccc(C(=O)O)nc1CN1CCOCCOCCN(Cc2cccc(C(=O)O)c2)CCOCCOCC1)NCCN(CCCN(CCNC(=O)CCc1ccc(C(=O)O)nc1CN1CCOCCOCCN(Cc2cccc(C(=O)O)c2)CCOCCOCC1)CCNC(=O)CCc1ccc(C(=O)O)nc1CN1CCOCCOCCN(Cc2cccc(C(=O)O)n2)CCOCCOCC1)CCNC(=O)CCc1ccc(C(=O)O)nc1CN1CCOCCOCCN(Cc2cccc(C(=O)O)n2)CCOCCOCC1]

The title compound can be obtained by using the methods described for Examples 8 and 9 above.

### Example 12 (Tet 7)

3,3′-[7,11-bis(2-{3-[2-carboxy-6-({16-[(6-carboxypyridin-2-yl)methyl]-1,4,10,13-tetraoxa-7,16-diazacyclooctadecan-7-yl}methyl)pyridin-3-yl]propanamido}ethyl)-3,15-dioxo-4,7,11,14-tetraazaheptadecane-1,17-diyl]bis[6-({16-[(6-carboxypyridin-2-yl)methyl]-1,4,10,13-tetraoxa-7,16-diazacyclooctadecan-7-yl}methyl)pyridine-2-carboxylic acid]

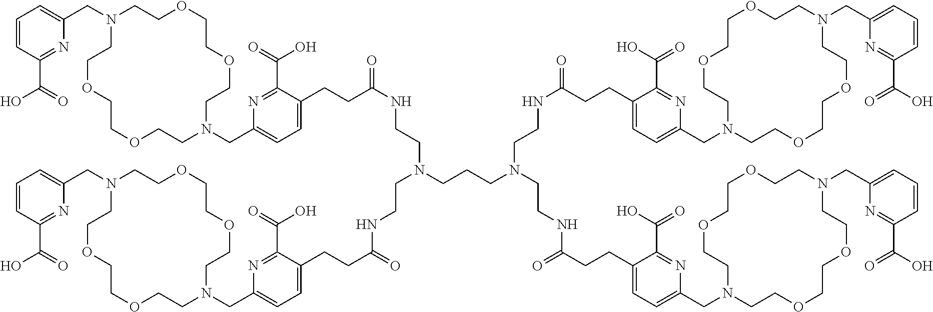 [SMILES: O=C(CCc1ccc(CN2CCOCCOCCN(Cc3cccc(C(=O)O)n3)CCOCCOCC2)nc1C(=O)O)NCCN(CCCN(CCNC(=O)CCc1ccc(CN2CCOCCOCCN(Cc3cccc(C(=O)O)n3)CCOCCOCC2)nc1C(=O)O)CCNC(=O)CCc1ccc(CN2CCOCCOCCN(Cc3cccc(C(=O)O)n3)CCOCCOCC2)nc1C(=O)O)CCNC(=O)CCc1ccc(CN2CCOCCOCCN(Cc3cccc(C(=O)O)n3)CCOCCOCC2)nc1C(=O)O]

The title compound can be obtained by using the methods described for Examples 8 and 9 above.

### Octameric Chelators

### Example 13 (Oct1)

methyl 4-[3-[[6-[2-[3-[bis[2-[2,6-bis[3-[2-methoxycarbonyl-6-[[16-[(6-methoxycarbonyl-2-pyridyl)methyl]-1,4,10,13-tetraoxa-7,16-diazacyclooctadec-7-yl]methyl]-4-pyridyl]propanoylamino]hexanoylamino]ethyl]amino]propyl-[2-[2,6-bis[3-[2-methoxycarbonyl-6-[[16-[(6-methoxycarbonyl-2-pyridyl)methyl]-1,4,10,13-tetraoxa-7,16-diazacyclooctadec-7-yl]methyl]-4-pyridyl]propanoylamino]hexanoylamino]ethyl]amino]ethylamino]-5-[3-[2-methoxycarbonyl-6-[[16-[(6-methoxycarbonyl-2-pyridyl)methyl]-1,4,10,13-tetraoxa-7,16-diazacyclooctadec-7-yl]methyl]-4-pyridyl]propanoylamino]-6-oxo-hexyl]amino]-3-oxo-propyl]-6-[[16-[(6-methoxycarbonyl-2-pyridyl)methyl]-1,4,10,13-tetraoxa-7,16-diazacyclooctadec-7-yl]methyl]pyridine-2-carboxylate

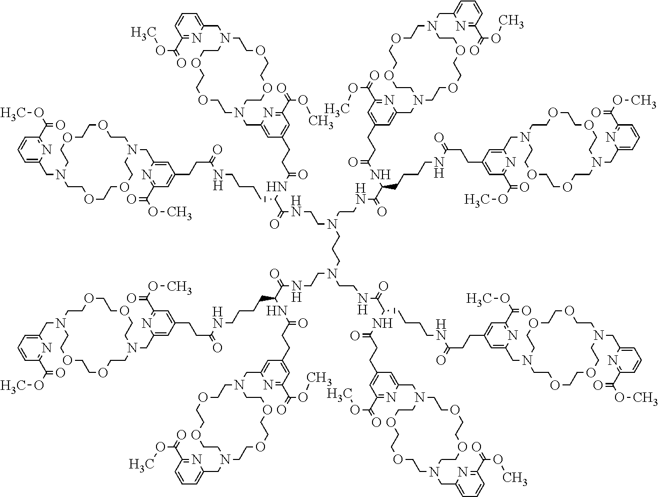

(2S)-2,6-diamino-N-[2-[3-[bis[2-[[(2S)-2,6-diaminohexanoyl]amino]ethyl]amino]propyl-[2-[[(2S)-2,6-diaminohexanoyl]amino]ethyl]amino]ethyl]hexanamide (5 mg, Intermediate 2), [2-({2-(methoxycarbonyl)-6-[(16-{[6-(methoxycarbonyl)pyridin-2-yl]methyl}-1,4,10,13-tetraoxa-7,16-diazacyclooctadecan-7-yl)methyl]pyridin-4-yl}amino)-2-oxoethoxy]acetic acid (15 mg, Intermediate 9) and PyAOP (12.4 mg) were dissolved in NMP (1 mL). DIPEA (16.6 μL) was added and reaction left for 1 hour. Reaction mixture was diluted with water/0.1% TFA (8 mL) and the product purified by preparative HPLC (column: Phenomenex Luna 5 μm C18(2) 100Å, 250×50 mm; gradient: 10-40% B over 40 min where A=water/0.1% TFA and B=ACN; flow: 10 mL/min; detection: UV 214/254 nm) affording 12.2 mg (78% yield) of the target compound after freeze-drying. Purified product was analyzed by analytical HPLC (gradient: 10-40% B over 2.5 min where A=water/0.1(% TFA and B=ACN, flow rate: 0.5 mL/min, column: Waters Acquity BEH C18, 1.7 μm, 2.1×50 mm, detection: UV diode array, product retention time: 1.77 min). Further product characterization was carried out using electrospray mass spectrometry (MH₂²⁺: 2837.5, found m/z: 2837.5).

### Example 14 (Oct2)

4-[3-[[6-[2-[3-[bis[2-[2,6-bis[3-[2-carboxy-6-[[16-[(6-carboxy-2-pyridyl)methyl]-1,4,10,13-tetraoxa-7,16-diazacyclooctadec-7-yl]methyl]-4-pyridyl]propanoylamino]hexanoylamino]ethyl]amino]propyl-[2-[2,6-bis[3-[2-carboxy-6-[[16-[(6-carboxy-2-pyridyl)methyl]-1,4,10,13-tetraoxa-7,16-diazacyclooctadec-7-yl]methyl]-4-pyridyl]propanoylamino]hexanoylamino]ethyl]amino]ethylamino]-5-[3-[2-carboxy-6-[[16-[(6-carboxy-2-pyridyl)methyl]-1,4,10,13-tetraoxa-7,16-diazacyclooctadec-7-yl]methyl]-4-pyridyl]propanoylamino]-6-oxo-hexyl]amino]-3-oxo-propyl]-6-[[16-[(6-carboxy-2-pyridyl)methyl]-1,4,10,13-tetraoxa-7,16-diazacyclooctadec-7-yl]methyl]pyridine-2-carboxylic acid

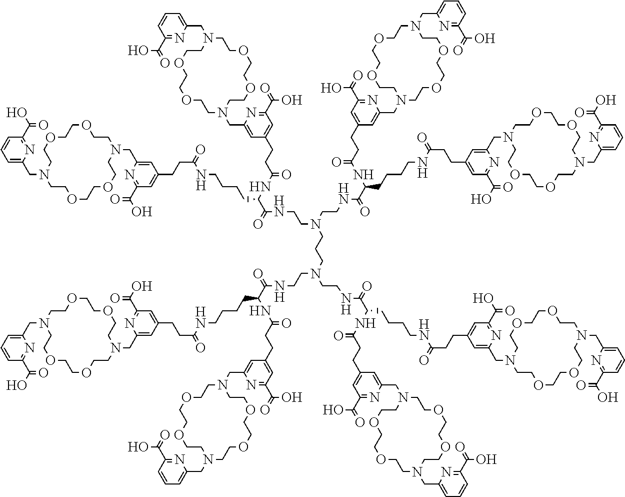

methyl 4-[3-[[6-[2-[3-[bis[2-[2,6-bis[3-[2-methoxycarbonyl-6-[[16-[(6-methoxycarbonyl-2-pyridyl)methyl]-1,4,10,13-tetraoxa-7,16-diazacyclooctadec-7-yl]methyl]-4-pyridyl]propanoylamino]hexanoylamino]ethyl]amino]propyl-[2-[2,6-bis[3-[2-methoxycarbonyl-6-[[16-[(6-methoxycarbonyl-2- pyridyl)methyl]-1,4,10,13-tetraoxa-7,16-diazacyclooctadec-7-yl]methyl]-4-pyridyl]propanoylamino]hexanoylamino]ethyl]amino]ethylamino]-5-[3-[2-methoxycarbonyl-6-[[16-[(6-methoxycarbonyl-2-pyridyl)methyl]-1,4,10,13-tetraoxa-7,16-diazacyclooctadec-7-yl]methyl]-4-pyridyl]propanoylamino]-6-oxo-hexyl]amino]-3-oxo-propyl]-6-[[16-[(6-methoxycarbonyl-2-pyridyl)methyl]-1,4,10,13-tetraoxa-7,16-diazacyclooctadec-7-yl]methyl]pyridine-2-carboxylate (12.2 mg, Example 13) was dissolved in water (2 mL). 5 M NaOH (100 μL) was added and reaction left for 1 hour. Reaction mixture was diluted with 10% ACN/water/0.1% TFA (8.5 mL) and the product purified by preparative HPLC (column: Phenomenex Luna 5 μm C18(2) 100Å, 250×50 mm; gradient: 10-30% B over 40 min where A=water/0.1% TFA and B=ACN; flow: 10 mL/min; detection: UV 214/254 nm) affording 7.5 mg (61% yield) of the target compound after freeze-drying. Purified product was analyzed by analytical HPLC (gradient: 10-30% B over 2.5 min where A=water/0.1% TFA and B=ACN, flow rate: 0.5 mL/min, column: Waters Acquity BEH C18, 1.7 μm, 2.1×50 mm, detection: UV diode array, product retention time: 1.65 min). Further product characterization was carried out using electrospray mass spectrometry (MH₂²⁺: 2725.4, found m/z: 2725.4).

### Chelator Active Esters

### Example 15 (AE1)

Bis(2,3,5,6-tetrafluorophenyl) 6,6′-[1,4,10,13-tetraoxa-7,16-diazacyclooctadecane-7,16-diylbis(methylene)]dipyridine-2-carboxylate

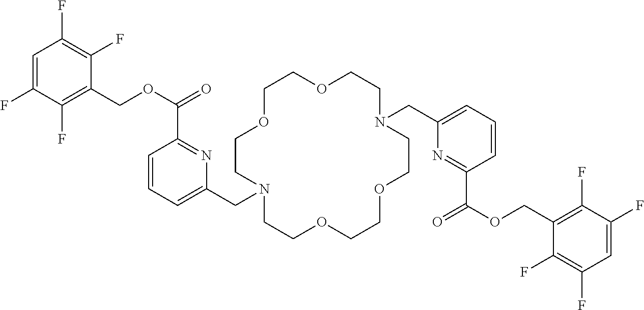 [SMILES: O=C(OCc1c(F)c(F)cc(F)c1F)c1cccc(CN2CCOCCOCCN(Cc3cccc(C(=O)OCc4c(F)c(F)cc(F)c4F)n3)CCOCCOCC2)n1]

6,6′-[1,4,10,13-tetraoxa-7,16-diazacyclooctadecane-7,16-diylbis(methylene)]dipyridine-2-carboxylic acid (30 mg, prepared as described in Angewandte Chemie, Nikki et al, 2017), TFP (47 mg) and DCC (35 mg) were dissolved in DCM (1 mL) and solution left for 20 hours. DCM was removed by a stream of air and the residue dissolved in ACN (2 mL), diluted with water/0.1% TFA (7 mL), filtered and product purified by preparative HPLC (column: Phenomenex Luna 5 μm C18(2) 100 Å, 250×50 mm; gradient: 20-70% B over 40 min where A=water/0.1% TFA and B=ACN; flow: 10 mL/min; detection: UV 214/254 nm) to afford 41 mg (88% yield) of the target compound after freeze-drying. Purified product was analyzed by analytical HPLC (gradient: 20-70% B over 2.5 min where A=water/0.1% TFA and B=ACN, flow rate: 0.5 mL/min, column: Waters Acquity BEH C18, 1.7 μm, 2.1×50 mm, detection: UV diode array, product retention time: 1.63 min). Further product characterization was carried out using electrospray mass spectrometry (MH⁺: 829.2, found m/z: 829.2).

### Example 16 (AE2)

6-({16-[(6-([16-({6-[(2,3,5,6-tetrafluorophenoxy)carbonyl]pyridin-2-yl}methyl)-1,4,10,13-tetraoxa-7,16-diazacyclooctadecan-7-yl]methyl}pyridin-2-yl)methyl]-1,4,10,13-tetraoxa-

[symbol]

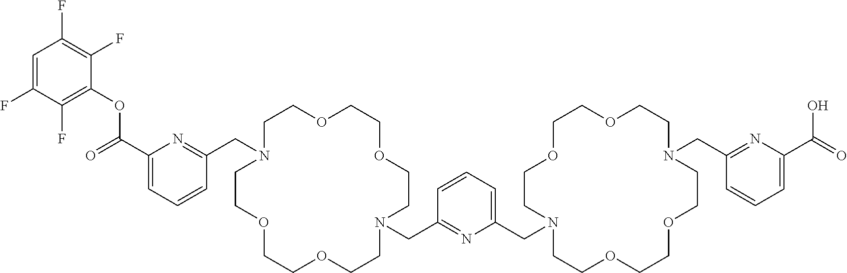 [SMILES: O=C(O)c1cccc(CN2CCOCCOCCN(Cc3cccc(CN4CCOCCOCCN(Cc5cccc(C(=O)Oc6c(F)c(F)cc(F)c6F)n5)CCOCCOCC4)n3)CCOCCOCC2)n1]

6,6′-[pyridine-2,6-diylbis(methylene-1,4,10,13-tetraoxa-7,16-diazacyclooctadecane-16,7-diylmethylene)]dipyridine-2-carboxylic acid (10 mg, Example 2), TFP (9.2 mg) and DCC (5.7 mg) were dissolved in DCM (1 mL) and solution left for20 hours. DCM was removed by a stream of air and the residue dissolved in ACN (1 mL), diluted with water/0.1% TFA (7.5 mL), filtered aid product purified by preparative HPLC (column: Phenomenex Luna 5 μm C18(2) 100 Å, 250×50 mm; gradient: 10-50% B over 40 min where A=water/0.1% TFA and B=ACN; flow: 10 mL/min; detection: UV 214/254 nm) to afford 1.2 mg (10% yield) of the target compound after freeze-drying. Purified product was analyzed by analytical HPLC (gradient: 10-50% B over 2.5 min where A=water/0.1% TFA and B=ACN, flow rate: 0.5 mL/min, column: Waters Acquity BEH C18, 1.7 μm, 2.1×50 mm, detection: UV diode array, product retention time: 1.52 min). Further product characterization was carried out using electrospray mass spectrometry (MH⁺: 1046.5, found m/z: 1046.5).

### Example 17 (AE3)

Methyl 4-[[2-[2-[2-[2-[[2-[2-[[2-methoxycarbonyl-6-[[16-[(6-methoxycarbonyl-2-pyridyl)methyl]-1,4,10,13-tetraoxa-7,16-diazacyclooctadec-7-yl]methyl]-4-pyridyl]amino]- 2-oxo-ethoxy]acetyl]amino]ethyl-[3-[2-[[2-[2-[[2-methoxycarbonyl-6-[[16-[(6-methoxycarbonyl-2-pyridyl)methyl]-1,4,10,13-tetraoxa-7,16-diazacyclooctadec-7-yl]methyl]-4-pyridyl]amino]-2-oxo-ethoxy]acetyl]amino]ethyl-[2-[[2-[2-oxo-2-(2,3,5,6-tetrafluorophenoxy)ethoxy]acetyl]amino]ethyl]amino]propyl]amino]ethylamino]-2-oxo-ethoxy]acetyl]amino]-6-[[16-[(6-methoxycarbonyl-2-pyridyl)methyl]-1,4,10,13-tetraoxa-7,16-diazacyclooctadec-7-yl]methyl]pyridine-2-carboxylate

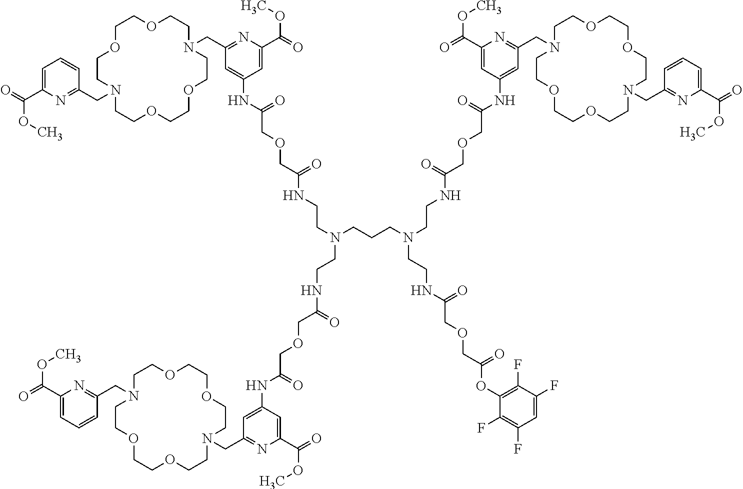 [SMILES: COC(=O)c1cccc(CN2CCOCCOCCN(Cc3cc(NC(=O)COCC(=O)NCCN(CCCN(CCNC(=O)COCC(=O)Nc4cc(CN5CCOCCOCCN(Cc6cccc(C(=O)OC)n6)CCOCCOCC5)nc(C(=O)OC)c4)CCNC(=O)COCC(=O)Oc4c(F)c(F)cc(F)c4F)CCNC(=O)COCC(=O)Nc4cc(CN5CCOCCOCCN(Cc6cccc(C(=O)OC)n6)CCOCCOCC5)nc(C(=O)OC)c4)cc(C(=O)OC)n3)CCOCCOCC2)n1]

21-({2-(methoxycarbonyl)-6-[(16-{[6-(methoxycarbonyl)pyridin-2-yl]methyl}-1,4,10,13-tetraoxa-7,16-diazacyclooctadecan-7-yl)methyl]pyridin-4-yl}amino)-9,13-bis(2-{2-[2-({2-(methoxycarbonyl)-6-[(16-{[6-(methoxycarbonyl)pyridin-2-yl]methyl}-1,4,10,13-tetraoxa-7,16-diazacyclooctadecan-7-yl)methyl]pyridin-4-yl}amino)-2-oxoethoxy]acetamido}ethyl)-5,17,21-trioxo-3,19-dioxa-6,9,13,16-tetraazahenicosan-1-oic acid (6.2 mg, Example 5), TFP (2.2 mg) and DCC (5.4 mg) were dissolved in DCM (1 mL) and solution left for 19 hours. DCM was removed by a stream of air and the residue dissolved in ACN (1 mL), diluted with water/0.1% TFA (7.5 mL), filtered and product purified by preparative HPLC (column: Phenomenex Luna 5 μm C18(2) 100 Å, 250×50 mm; gradient: 10-50% B over 40 min where A=water/0.1% TFA and B=ACN; flow: 10 mL/min; detection: UV 214/254 nm) to afford 2.2 mg (33% yield) of the target compound after freeze-drying. Purified product was analyzed by analytical HPLC (gradient: 10-50% B over 2.5 min where A=water/0.1% TFA and B=ACN, flow rate: 0.5 mL/min, column: Waters Acquity BEH C18, 1.7 μm, 2.1×50 mm, detection: UV diode array, product retention time: 1.58 min). Further product characterization was carried out using electrospray mass spectrometry (MH⁺: 2531.1, found m/z: 2531.2).

### Example 18 (AE4)

4-[3-[2-[2-[3-[2-carboxy-6-[[16-[(6-carboxy-2-pyridyl)methyl]-1,4,10,13-tetraoxa-7,16-diazacyclooctadec-7-yl]methyl]-4-pyridyl]propanoylamino]ethyl-[3-[2-[3-[2-carboxy-6-[[16-[(6-carboxy-2-pyridyl)methyl]-1,4,10,13-tetraoxa-7,16-diazacyclooctadec-7-yl]methyl]-4-pyridyl]propanoylamino]ethyl-[2-[3-[2-carboxy-6-[[16-[(6-(2,3,5,6-

[symbol]

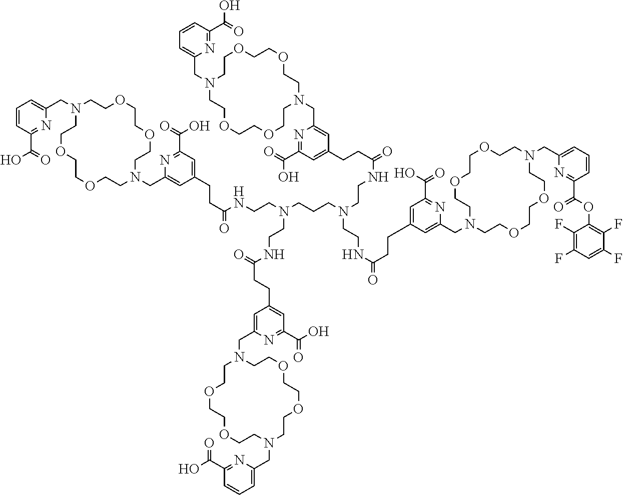 [SMILES: O=C(CCc1cc(CN2CCOCCOCCN(Cc3cccc(C(=O)O)n3)CCOCCOCC2)nc(C(=O)O)c1)NCCN(CCCN(CCNC(=O)CCc1cc(CN2CCOCCOCCN(Cc3cccc(C(=O)O)n3)CCOCCOCC2)nc(C(=O)O)c1)CCNC(=O)CCc1cc(CN2CCOCCOCCN(Cc3cccc(C(=O)Oc4c(F)c(F)cc(F)c4F)n3)CCOCCOCC2)nc(C(=O)O)c1)CCNC(=O)CCc1cc(CN2CCOCCOCCN(Cc3cccc(C(=O)O)n3)CCOCCOCC2)nc(C(=O)O)c1]

4,4′-[7,11-bis(2-{3-[2-carboxy-6-({16-[(6-carboxypyridin-2-yl)methyl]-1,4,10,13-tetraoxa-7,16-diazacyclooctadecan-7-yl}methyl)pyridin-4-yl]propanamido}ethyl)-3,15-dioxo-4,7,11,14-tetraazaheptadecane-1,17-diyl]bis[6-({16-[(6-carboxypyridin-2-yl)methyl]-1,4,10,13-tetraoxa-7,16-diazacyclooctadecan-7-yl}methyl)pyridine-2-carboxylic acid] (19.2 mg, Example 10), TFP (24.6 mg) and DCC (12.7 mg) were dissolved in ACN (1 mL) and solution leftfor 30 min. Solution was diluted with water/0.1% TFA (9 mL), filtered and product purified by preparative HPLC (column: Phenomenex Luna 5 μm C18(2) 100 Å, 250×50 mm; gradient: 10-60% B over 40 min where A=water/0.1% TFA and B=ACN; flow: 10 mL/min; detection: UV 214/254 nm) to afford 6 mg (28% yield) of the target compound after freeze-drying. Purified product was analyzed by analytical HPLC (gradient: 10-70% B over 2.5 min where A=water/0.1% TFA and B=ACN, flow rate: 0.5 mL/min, column: Waters Acquity BEH C18, 1.7 μm, 2.1×50 mm, detection: UV diode array, product retention time: 1.04 and 1.13 min (mixture of two regioisomers)). Further product characterization was carried out using electrospray mass spectrometry (MH⁺: 2740.3, found m/z: 2740.2).

### Example 19 (AE5)

4-[3-[[6-[2-[2-[2,6-bis[3-[2-carboxy-6-[[16-[(6-carboxy-2-pyridyl)methyl]-1,4,10,13-tetraoxa -7,16-diazacyclooctadec-7-yl]methyl]-4-pyridyl]propanoylamino]hexanoylamino]ethyl-[3-[2-[2,6-bis[3-[2-carboxy-6-[[16-[(6-carboxy-2-pyridyl)methyl]-1,4,10,13-tetraoxa-7,16-diazacyclooctadec-7-yl]methyl]-4-pyridyl]propanoylamino]hexanoylamino]ethyl-[2-[[2-[3-[2-carboxy-6-[[16-[(6-carboxy-2-pyridyl)methyl]-1,4,10,13-tetraoxa-7,16-diazacyclooctadec-7-yl]methyl]-4-pyridyl]propanoylamino]-6-[3-[2-carboxy-6-[[16-[[6-(2,3,5,6-tetrafluorophenoxy)carbonyl-2-pyridyl]methyl]-1,4,10,13-tetraoxa-7,16-diazacyclooctadec-7-yl]methyl]-4-pyridyl]propanoylamino]hexanoyl]amino]ethyl]amino]propyl]amino]ethylamino]-5-[3-[2-carboxy-6-[[16-[(6-carboxy-2-pyridyl)methyl]-1,4,10,13-tetraoxa-7,16-diazacyclooctadec-7-yl]methyl]-4-pyridyl]propanoylamino]-6-oxo-hexyl]amino]-3-oxo-propyl]-6-[[16-[(6-carboxy-2-pyridyl)methyl]-1,4,10,13-tetraoxa-7,16-diazacyclooctadec-7-yl]methyl]pyridine-2-carboxylic acid

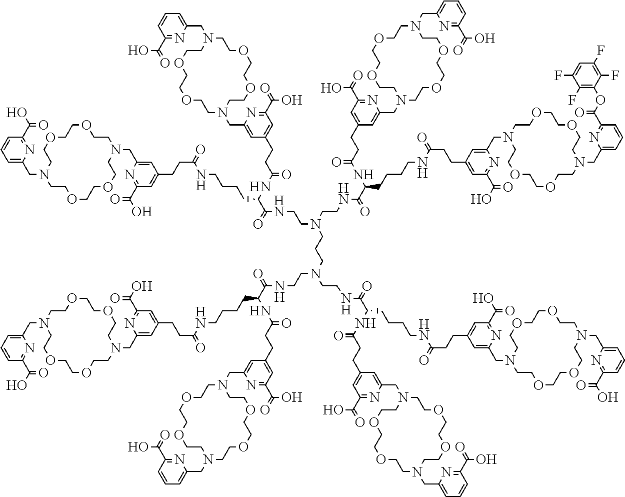

4-[3-[[6-[2-[3-[bis[2-[2,6-bis[3-[2-carboxy-6-[[16-[(6-carboxy-2-pyridyl)methyl]-1,4,10,13-tetraoxa-7,16-diazacyclooctadec-7-yl]methyl]-4-pyridyl]propanoylamino]hexanoylamino]ethyl]amino]propyl-[2-[2,6-bis[3-[2-carboxy-6-[[16-[(6-carboxy-2-pyridyl)methyl]-1,4,10,13-tetraoxa-7,16-diazacyclooctadec-7-yl]methyl]-4-pyridyl]propanoylamino]hexanoylamino]ethyl]amino]ethylamino]-5-[3-[2-carboxy-6-[[16-[(6-carboxy-2-pyridyl)methyl]-1,4,10,13-tetraoxa-7,16-diazacyclooctadec-7-yl]methyl]-4-pyridyl]propanoylamino]-6-oxo-hexyl]amino]-3-oxo-propyl]-6-[[16-[(6-carboxy-2-pyridyl)methyl]-1,4,10,13-tetraoxa-7,16-diazacyclooctadec-7-yl]methyl]pyridine-2-carboxylic acid (3.8 mg, Example 14), TFP (6.3 mg) and DCC (2.3 mg) were dissolved in ACN (1 mL) and solution left for 30 min. Solution was diluted with water/0.1% TFA (9 mL), filtered and product purified by preparative HPLC (column: Phenomenex Luna 5 μm C18(2) 100 Å, 250×50 mm; gradient: 10-60% B over 40 min where A=water/0.1% TFA and B=ACN; flow: 10 mL/min; detection: UV 214/254 nm) to afford 0.9 mg (23% yield) of the target compound after freeze-drying. Purified product was analyzed by analytical HPLC (gradient: 10-60% B over 2.5 min where A=water/0.1% TFA and B=ACN, flow rate: 0.5 mL/min, column: Waters Acquity BEH C18, 1.7 μm, 2.1×50 mm, detection: UV diode array, product retention time: 1.20, 1.23 and 1.33 min (mixture of three regioisomers)). Further product characterization was carried out using electrospray mass spectrometry (MH₄⁴⁺: 1400.2, found m/z: 1400.4).

### Antibody-Chelator Conjugates

### Example 20 (ACC1)

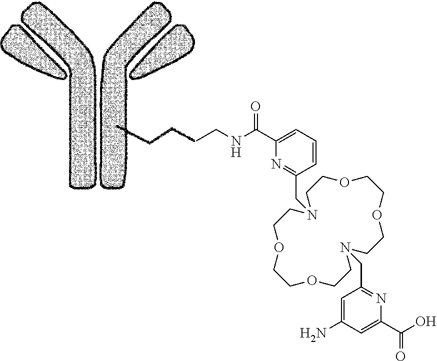 [SMILES: CCNC(=O)c1cccc(CN2CCOCCOCCN(Cc3cc(N)cc(C(=O)O)n3)CCOCCOCC2)n1]

Bis(2,3,5,6-tetrafluorophenyl) 6,6′-[1,4,10,13-tetraoxa-7,16-diazacyclooctadecane-7,16-diyIbis(methylene)]dipyridine-2-carboxylate (1.67 mg, Example 15) dissolved in DMA (84 μL) was added to mAb no. 1 (50.5 mg) in PBS (4 mL) and solution shaken for 4 hours. Solution was diluted with 100 mM acetate/100 mM NaCl 1:1 (1 mL) product purified by FPLC (column: HiLoad 16/600 Superdex 200 pg column; running buffer: 100 mM acetate/100 mM NaCl 1:1, pH 5; flow: 1 mL/min; detection: UV 214/254 nm) to afford 35.6 mg (71% yield) of ACC1 in 100 mM acetate/100 mM NaCl 1:1 (2.7 mg/mL).

### Example 21 (ACC2)

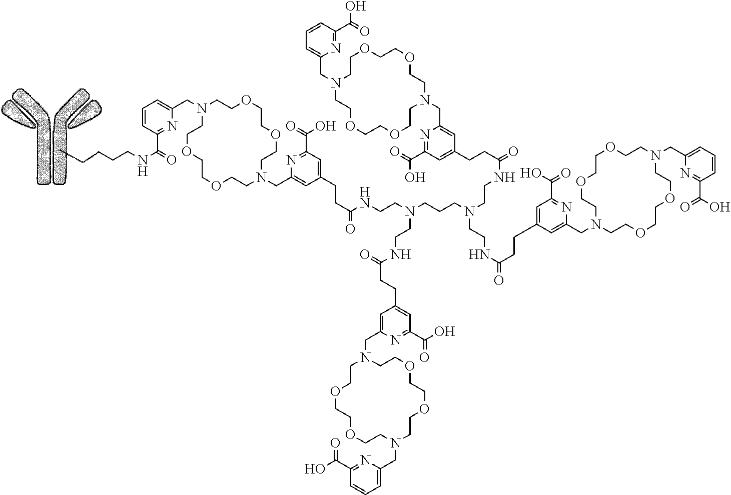 [SMILES: CCCNC(=O)c1cccc(CN2CCOCCOCCN(Cc3cc(CCC(=O)NCCN(CCCN(CCNC(=O)CCc4cc(CN5CCOCCOCCN(Cc6cccc(C(=O)O)n6)CCOCCOCC5)nc(C(=O)O)c4)CCNC(=O)CCc4cc(CN5CCOCCOCCN(Cc6cccc(C(=O)O)n6)CCOCCOCC5)nc(C(=O)O)c4)CCNC(=O)CCc4cc(CN5CCOCCOCCN(Cc6cccc(C(=O)O)n6)CCOCCOCC5)nc(C(=O)O)c4)cc(C(=O)O)n3)CCOCCOCC2)n1]

4-[3-[2-[2-[3-[2-carboxy-6-[[16-[(6-carboxy-2-pyridyl)methyl]-1,4,10,13-tetraoxa-7,16-diazacyclooctadec-7-yl]methyl]-4-pyridyl]propanoylamino]ethyl-[3-[2-[3-[2-carboxy-6-[[16-[(6-carboxy-2-pyridyl)methyl]-1,4,10,13-tetraoxa-7,16-diazacyclooctadec-7-yl]methyl]-4-pyridyl]propanoylamino]ethyl-[2-[3-[2-carboxy-6-[[16-[[6-(2,3,5,6-tetrafluorophenoxy)carbonyl-2-pyridyl]methyl]-1,4,10,13-tetraoxa-7,16-diazacyclooctadec-7-yl]methyl]-4-pyridyl]propanoylamino]ethyl]amino]propyl]amino]ethylamino]-3-oxo-propyl]-6-[[16-[(6-carboxy-2-pyridyl)methyl]-1,4,10,13-tetraoxa-7,16-diazacyclooctadec-7-yl]methyl]pyridine-2-carboxylic acid (1.79 mg, Example 18) was added to mAb no. 1 (20 mg) in PBS (1.79 mL) and solution shaken for 3 hours. Solution was diluted with 100 mM acetate/100 mM NaCl 1:1 (3.2 mL) product purified by FPLC (column: HiLoad 16/600 Superdex 200 pg column; running buffer 100 mM acetate/100 mM NaCl 1:1, pH 5; flow: 1 mL/min; detection: UV 214/254 nm) to afford 14 mg (70% yield) of ACC2 in 100 mM acetate/100 mM NaCl 1:1 (1.0 mg/mL).

Other ACCs were prepared using same procedure as for ACC1 and ACC2 starting from compounds as described in examples 15, 16, 17, 18, and 19.

Purity and concentration of the ACCs were determined by SEC-UV (Agilent 1260 Infinity HPLC system, running buffer: 10% DMSO/PBS; flow rate: 0.3 mL/min, column: Waters Acquity BEH SEC, 1.7 μm, 4.6×300 mm, detection: UV at 280 nm).

CAR for each of the ACCs was determined by SEC-MS (Water Acquity HPLC connected to Waters XEVO TOF; running buffer: 50% ACN/water/0.1% TFA; flow rate: 0.06 mL/min, column: Waters Acquity BEH SEC, 1.7 μm, 2.1×150 mm) by using MS peak heights in percentage of major peak height for the components mAb, mAb+1 chelator, mAb+2 chelator, mAb+3 chelator etc. and using the formula CAR=Sum(n*An)/Sum An, where n equals the number of chelators and An equals the intensity of the antibody conjugate with n chelators

TABLE 1
ACCs prepared

| | | | | | ACC | |
|---|---|---|---|---|---|---|
| | | | | | purity | Concentration |
| | | Batch | | | (% area at | of purified |
| mAb | Chelator | size | | CAR | 280 nM) | ACC (mg/mL) |
| mAb no. 2 | Macropa | 36 | mg | 1.4 | 99 | 2.4 |
| mAb no. 2 | Dim2 | 25 | mg | 0.9 | 99 | 2.0 |
| mAb no. 2 | Tri1 | 25 | mg | 0.2 | 99 | 1.8 |
| mAb no. 2 | Tet5 | 25 | mg | 0.5 | 99 | 1.9 |
| mAb no. 3 | Macropa | 20 | mg | 1.5 | 99 | 1.6 |
| mAb no. 3 | Tet5 | 25 | mg | 2.1 | 99 | 1.0 |
| mAb no. 3 | Tet5 | 21.5 | mg | 0.7 | 99 | 1.9 |
| Trastuzumab | Macropa | 50 | mg | 0.9 | 99 | 3.0 |
| mAb no. 1 | Macropa | 50 | mg | 1.4 | 99 | 2.7 |
| mAb no. 1 | Macropa | 50 | mg | 5.3 | 99 | 2.9 |
| mAb no. 1 | Dim2 | 20 | mg | 0.9 | 99 | 1.2 |
| mAb no. 1 | Tet5 | 25 | mg | 1.4 | 99 | 1.0 |
| mAb no. 1 | Tet5 | 25 | mg | 0.7 | 99 | 1.5 |
| mAb no. 4 | Oct2 | 20 | mg | 0.5 | 99 | 1.1 |
| Isotype | Macropa | 30 | mg | 1.1 | 99 | 2.6 |

### Radiolabeling

Aliquots of Ac-225 in 0.04 M HCl or Ra-223 in 0.05 M HCl were withdrawn into Eppendorf tubes. The radioactivity in each tube was measured by HPGe detector. Solutions of compounds in 0.1 M sodium acetate, pH 5-5.5 (with additional 0.1 M NaCl for ACC solutions) were added to the tubes. RAC was in the range of 1-5 M Bq/mL and specific activity in the range of 2-200 kBq/nmol. The labelling solutions were left for 60-90 min at room temperature.

### Radiochemical Purity

Radiochemical purity (RCP) of the labeled compounds was determined by iTLC. iTLC strips were cut from silica impregnated chromatography paper, approx. 1 cm wide and 11 cm long. The strips were marked with a pen at 1 cm (application point), 4 cm (cut line for ACCs) or 5 cm (cut line for chelators) and 8 cm (front line). A beaker was filled up to 0.5 cm with 0.1 M citrate in 0.9% NaCl, pH 5.5. 1-10 μL of the radiolabeled compound was added to the application point and the strips immediately placed vertically in the beaker. The strips were removed when the solvent front reached the front line and then cut in two sections at the cut line. Each section was measured using a HPGe detector (ORTEC) to determine the radioactivity origin from the nuclide of interest. The RCP, in percentage, forthe nuclide of interest was calculated using the following equation:

$$\% R C P = \frac{Radioactivity of a pplication section}{Total radioactivity ( application section + front section )} * 100$$

TABLE 2
RCP results by iTLC of radiolabeled compounds

| | | |
|---|---|---|
| | RCP Ra-223 | RCP Ac-225 |
| | (labelling | (labelling |
| Compound | concentration) | concentration) |

| | | | | |
|---|---|---|---|---|
| Macropa | 8% | (0.005 mM) | 100% | (0.02 mM) |
| Macropa-NH₂ | 12% | (0.27 mM) | 99% | (0.02 mM) |
| | 2% | (0.1 mM) | | |
| Dim1 | 80% | (0.02 mM) | 100% | (0.02 mM) |
| Dim2 | 11% | (0.2 mM) | 100% | (0.02 mM) |
| Dim3 | 32% | (0.2 mM) | 100% | (0.02 mM) |
| Tri1 | 89% | (0.02 mM) | | |
| Tet1 | 95% | (0.02 mM) | 99% | (0.02 mM) |
| Tet2 | 74% | (0.02 mM) | | |
| Tet3 | 95% | (0.02 mM) | 91% | (0.02 mM) |
| Tet5 | 95% | (0.02 mM) | | |
| | 36% | (0.005 mM) | | |
| Oct2 | 64% | (0.005 mM) | | |
| mAb no. 1-macropa | 33% | (0.02 mM) | 99% | (0.02 mM) |
| mAb no. 1-Tet5 | 37% | (0.02 mM) | 99% | (0.02 mM) |
| mAb no. 3-macropa | 8% | (0.02 mM) | 100% | (0.02 mM) |
| mAb no. 3-Tet5 | 65% | (0.02 mM) | 96% | (0.02 mM) |
| mAb no. 3-Oct2 | 51% | (0.02 mM) | | |
| mAb no. 2-macropa | 38% | (0.02 mM) | 100% | (0.02 mM) |
| mAb no. 2-Tri1 | 64% | (0.02 mM) | 81% | (0.02 mM) |
| mAb no. 2-Tet5 | | | 100% | (0.02 mM) |

Multimeric compounds Dim1, Tri1, Tet1, Tet2, Tet3, Tet5 and Oct2 demonstrated high labelling efficiency compared to monomeric macropa, at 0.1 and 0.02 mM concentrations, and even as low as 0.005 mM for Oct2 At these concentrations no complexation of radium-223 was observed to monomeric macropa and even at 0.27 mM only 12% radiochemical purity was obtained as measured by iTLC (table 2).

### Radio-HPLC

Radiolabeled compounds were analyzed by radio-HPLC using either a) Vanquish HPLC system (Thermo) equipped with a diode array detector and a Flowstar LB 514 radio detector (Berthold technologies); or b) an 1290 Infinity-II HPLC system (Agilent) equipped with a diode array detector and flow-count radio detector (Eckert & Ziegler).

Labelled chelator compounds were eluted using A=40 mM TRIS/6 mM citrate/2 mM EDTA and B=ACN/MeOH (8:2); aKinetexC18 (30×2.1 mm), 1.7 μm, 100 Å, Phenomenex); gradient 5-50% B for 10 min; flow rate of 0.3 mL/min or a Discovery RP amide c16 (150×2.1mm), 5 um, 100 Å, Gradient: 5-50% B for 12.5 min; flow rate of 0.6 mL/min.

Labelled ACCs were eluted using a Acquity Protein BEH SEC-column (300×4.6mm, 200 Å, Waters), and running buffer of 170 mM ammonium acetate/300 mM NaCl/5% DMSO, pH 5, using an isocratic flow of 0.3 mL/min for 20 min.

Chromeleon chromatography data system (CDS) was used for recording, integration and visualization of chromatograms.

Radio-HPLC peak fractioning was performed to determine the radionuclide(s) associated to each radio peak. The collected peak fractions were analysed using an HPGe detector.

Radio HPLC analysis of compound Teti demonstrated almost no wash through of free radioactivity in the void volume and a large radioactive peak with a retention time of 6-8 min corresponding to a complexes of Ra-223, Pb211 and Bi-211 (Figure). This very surprising observation points at the fact that the radium and daughters are captured in a far superior way by introducing multiple chelating agents most likely through contributions from donor atoms on adjacent chelators and/or avididy effect. Most interestingly the efficient labeling of a 0.02 mM solution of compound Tet1 is at the required level for enabling targeted alpha therapy at relevant ligand concentrations and doses.

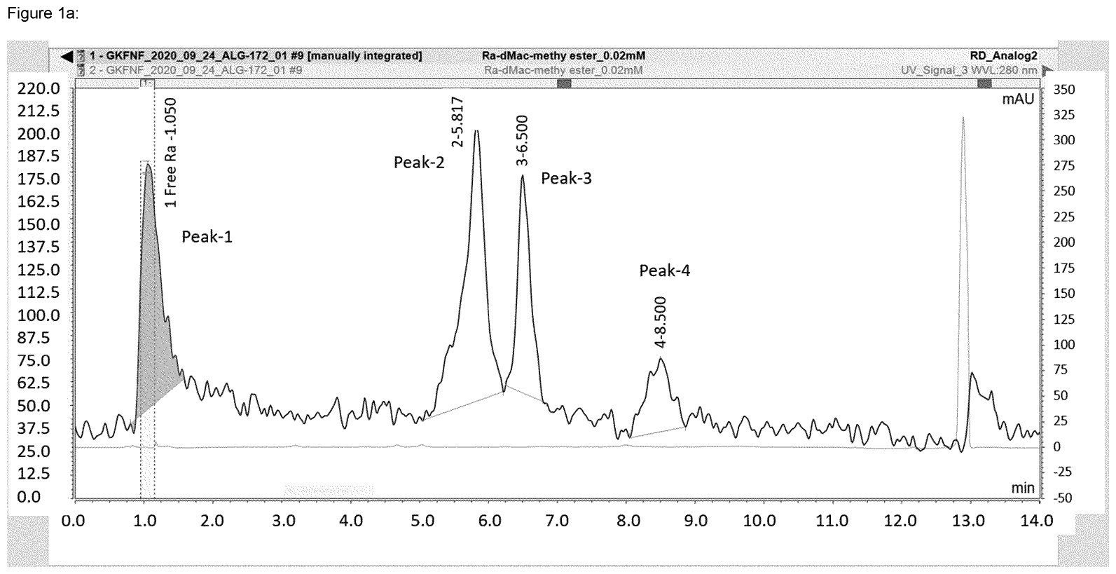

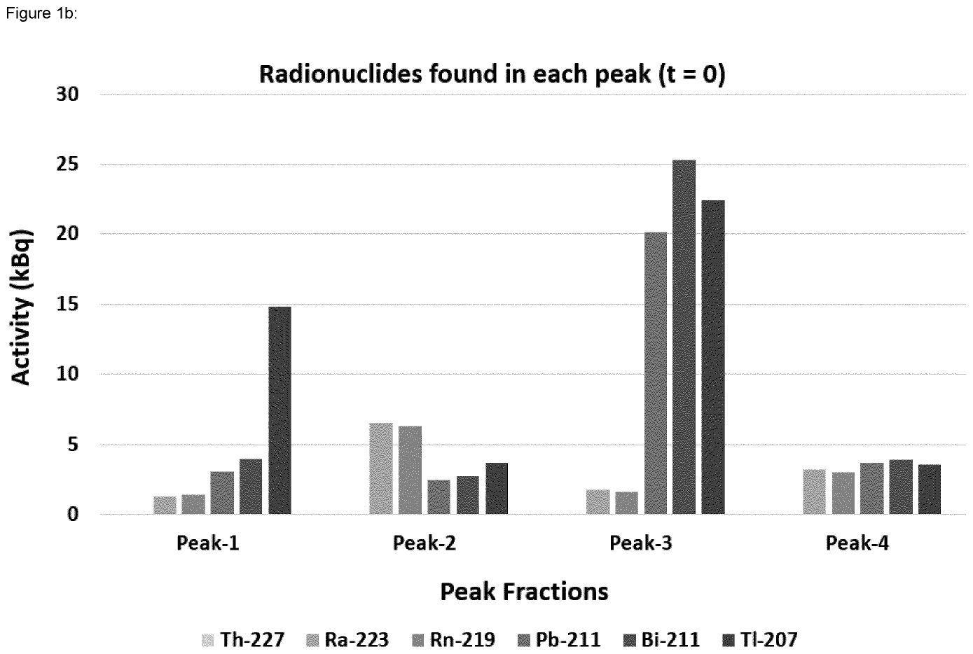

FIG. 1a illustrates radio HPLC chromatogram of ²²³Ra-Dim1 labeled at 0.02 mM concentration.

FIG. 1b illustrates peak fractioning data of ²²³Ra-Dim1 labeled at 0.02 mM concentration

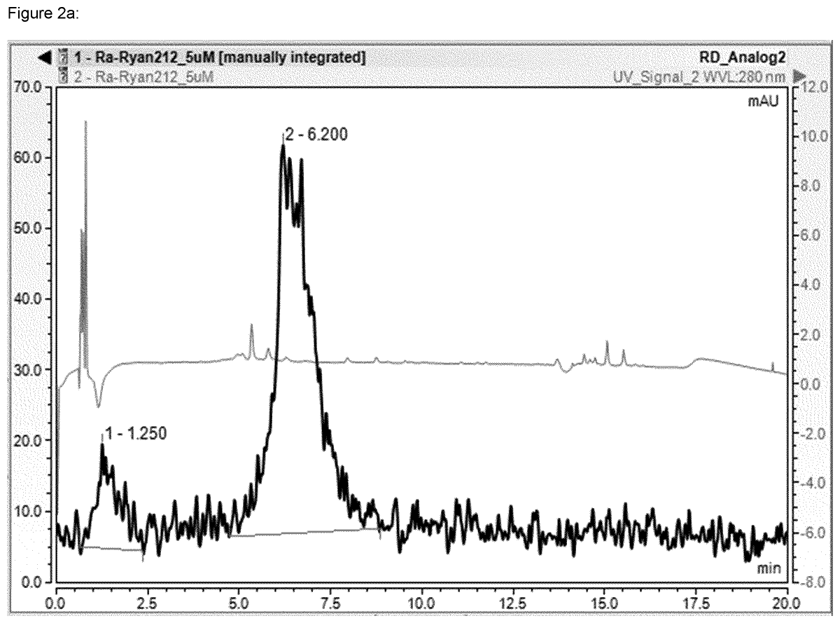

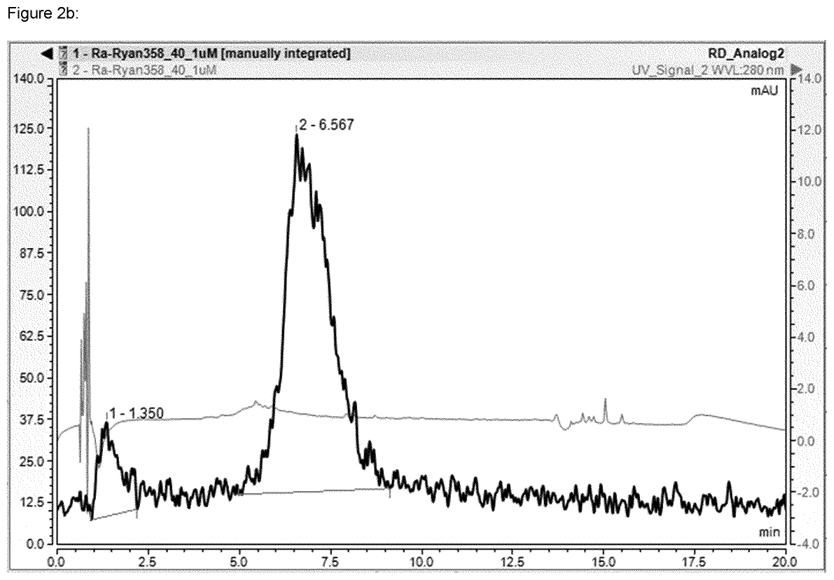

FIG. 2a illustrates radio HPLC chromatogram of ²²³Ra-Tet5 labeled at 0.005 mM concentration.

FIG. 2b illustrates radio HPLC chromatogram of ²²³Ra-Oct2 labeled at 0.001 mM concentration.

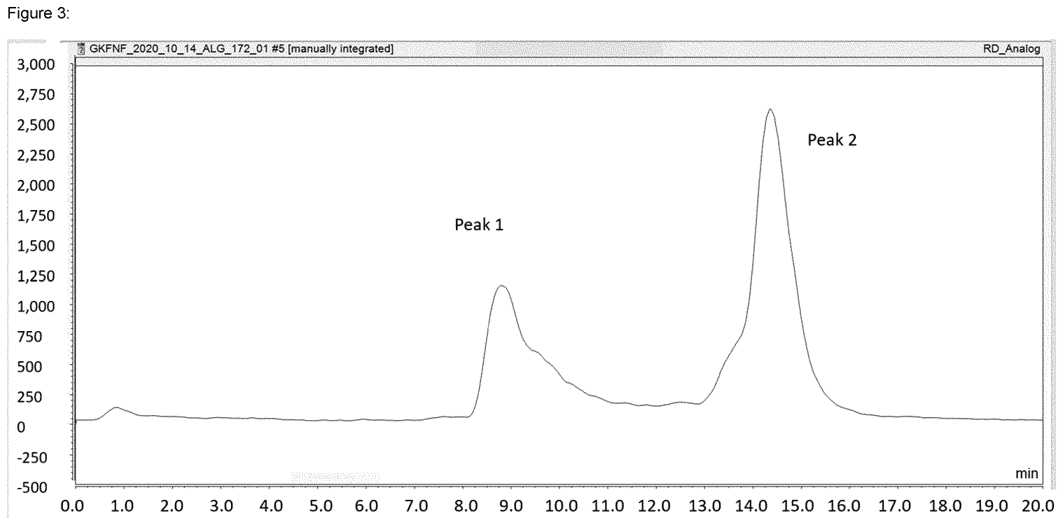

FIG. 3 illustrates radio HPLC chromatogram of ²²⁵Ac-mAb no. 1-macropa labeled at 0.02 mM concentration.

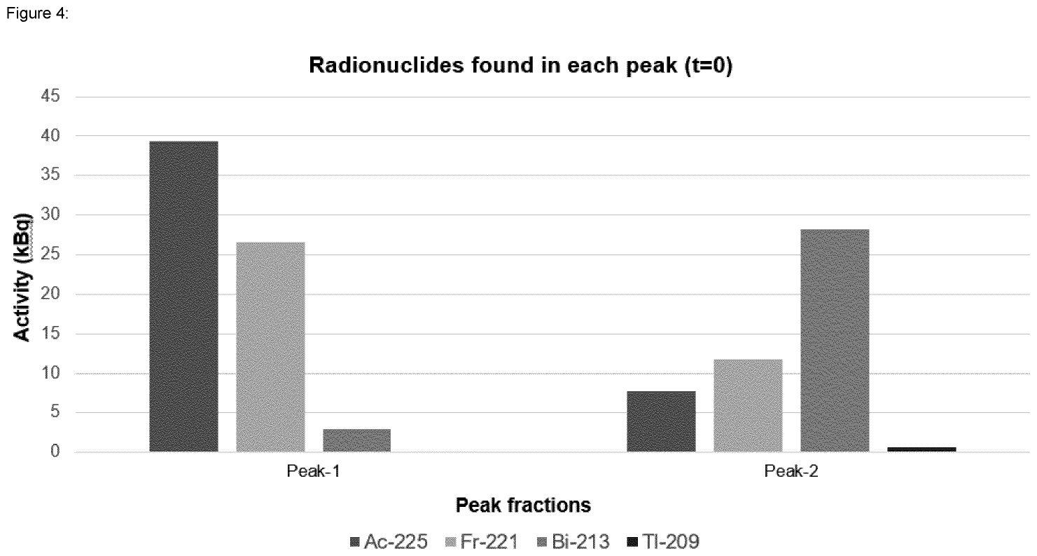

FIG. 4 illustrates peak fractioning data for ²²⁵Ac-mAb no. 1-macropa labeled at 0.02 mM.

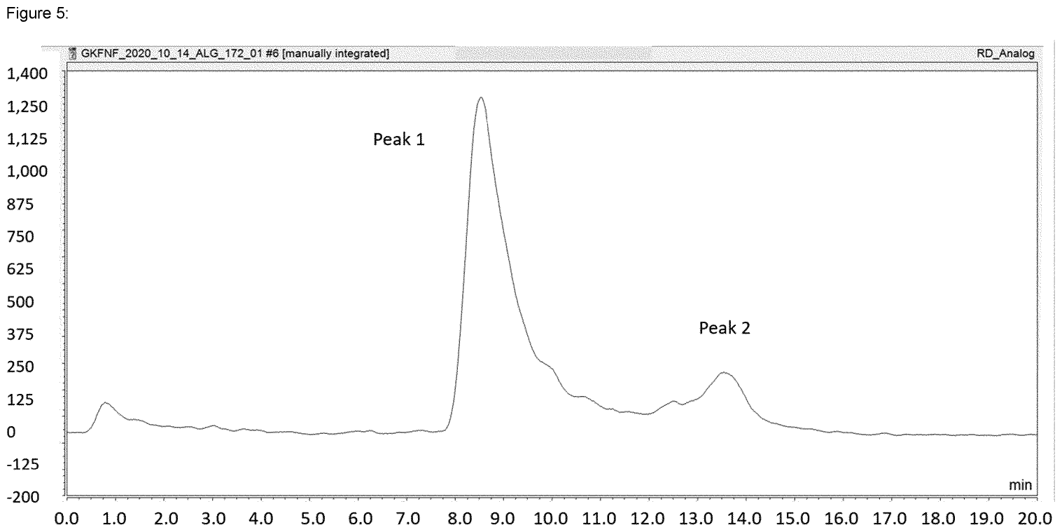

FIG. 5 illustrates radio HPLC chromatogram for ²²⁵Ac-mAb no. 1-Tet5 labeled at 0.02 mM.

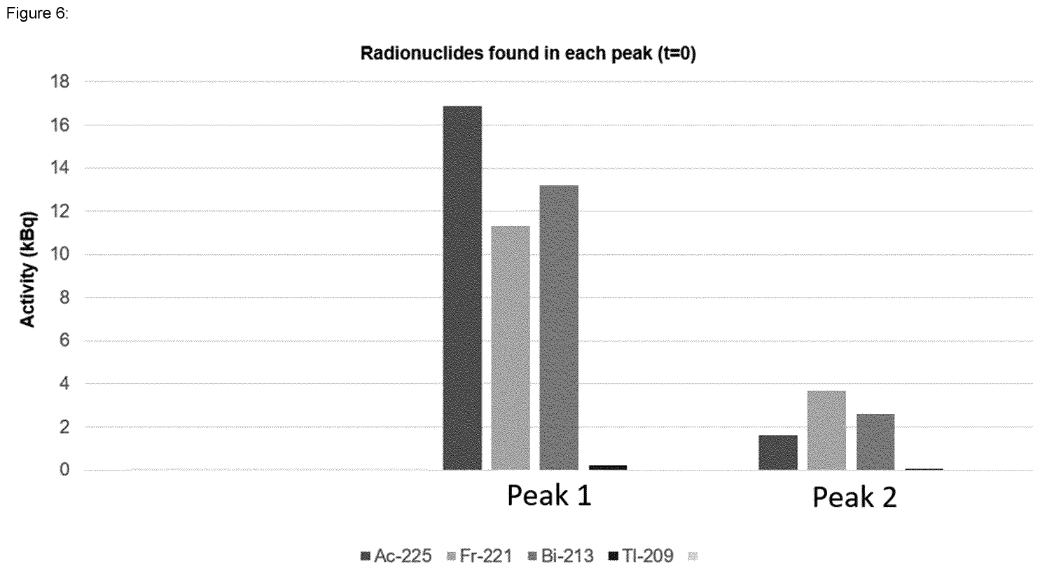

FIG. 6 illustrates peak fractioning data for ²²⁵Ac-mAb no. 1-Tet5 labeled at 0.02 mM.

### Experimental Section—Biological Assays

Examples were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values or median values calculated utilizing data sets obtained from testing of one or more synthetic batch.

### In Vitro

Antigen binding properties of Ac-225 labelled mAb no. 1-macropa (CAR 5.3) and mAb no. 1-Tet5 (CAR 1.4) was conducted using an IRF assay, whereby magnetic beads coated with the specific antigen were incubated with the radiolabelled compounds, allowing the bound fraction to be easily separated from the unbound supernatant fraction by magnetism. The unbound fraction was determined by sampling a representative 50% of the supernatant. Identical replicates pre-incubated with a target antigen specific binding site blocking agent, such as the non-radiolabelled naked mAb, was utilised to determine any non-specific binding of the radiolabelled product in the assay. The radioactivity in each sample was determined using a HPGe detector. Together these values provided the specific binding value and thus the IRF (specifically bound radiolabelled product expressed as a percentage of the total radiolabelled product applied).

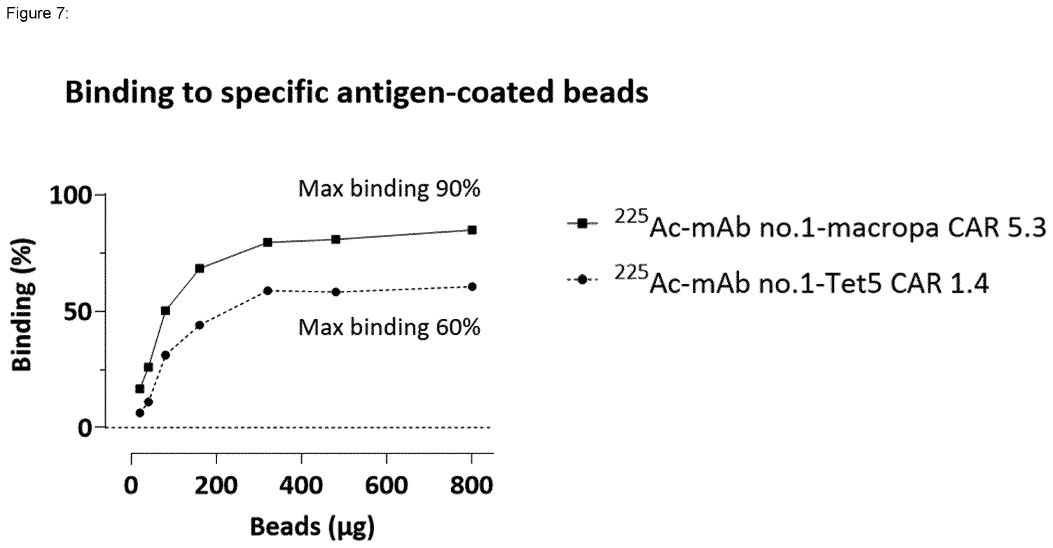

FIG. 7 illustrates binding curves and max binding IRF values for Ac-225 labelled mAb no. 1-macropa (CAR 5.3) and mAb no. 1-Tet5 (CAR 1.4).

Serum stability of Ac-225 labelled mAb no. 2-macropa, mAb no. 2-Tri1 and mAb no. 2-Tet5 was investigated by adding 25 kBq/mL of the labelled compounds to mouse serum and incubating at 37° C. with gentle shaking. The RCP of the labelled compounds was measured by iTLC after 1 hour, 96 hours, 120 hours and 144 hours. Percentage of the RCP at labelling (1 hour time point) was displayed for each time point.

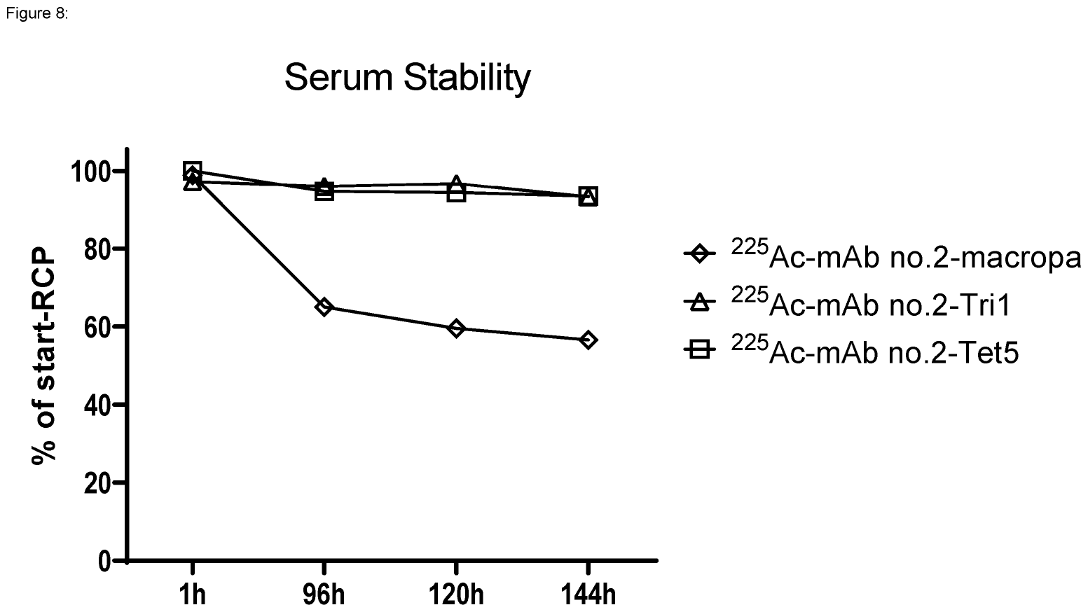

FIG. 8 illustrates serum stability of Ac-225 labelled mAb no. 2-macropa, mAb no. 2-Tri1 and mAb no. 2-Tet5.

### In Vivo

A biodistribution study of Ra-223 labelled Macropa-NH₂ and Tet1 was conducted. The compounds were labeled with Ra-223 in 0.1 M acetate, pH 5, at 125 kBq/nmol and injected respectively in mice at 500 kBq/kg. Ra-223 acetate was injected separately as control. Animals were sacrificed after 5 min, 30 min, 4 hours and 24 hours, with three animals for each time point. Liver, blood and femur were collected for all animals and the samples counted using HPGe detector to determine the amount of Ra-223.

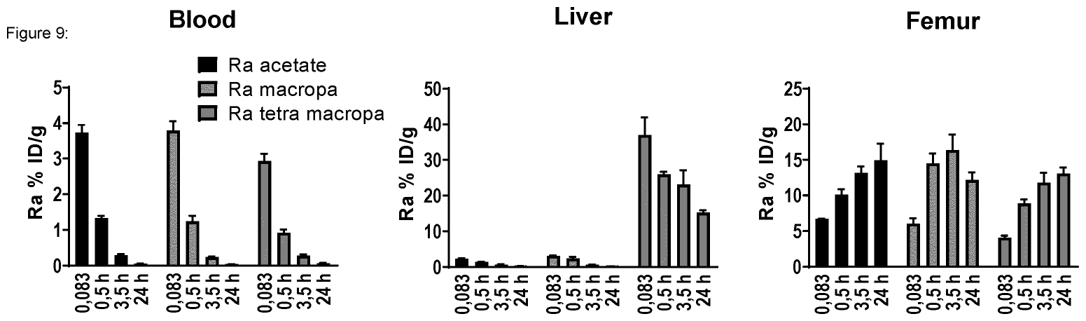

FIG. 9 illustrates percentage injected dose of ²²³Ra acetate, ²²³Ra-macropa-NH₂ and ²²³Ra-Tet1 per gram sample.

A biodistribution study of Ac-225 labelled mAb no. 3-macropa and mAb no. 3-Tet5 was conducted. The compounds were labeled with Ac-225 in 0.1 M acetate, pH 5, at 125 kBq/nmol and injected respectively in HEP-3B treated mice three times at 500 kBq/kg. Ac-225 acetate was injected separately as control. Animals were sacrificed after 24 hours, 72 hours, 168 hours aid 336 hours, three animals at each time point. Liver, blood and femur were collected for all animals.

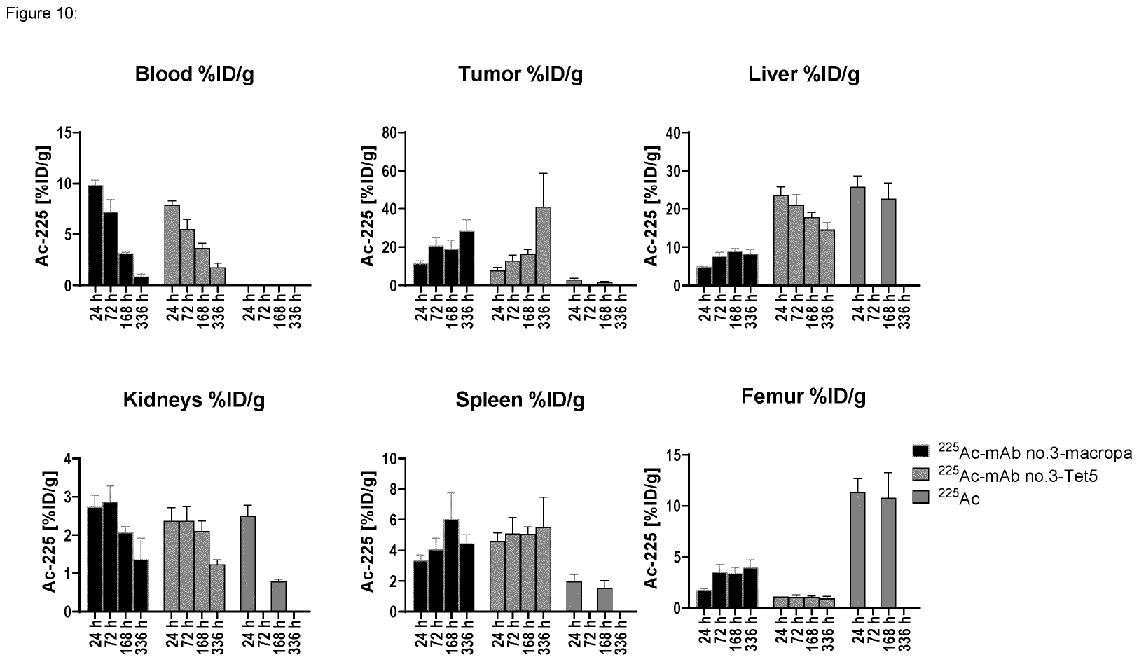

FIG. 10 illustrates percentage injected dose of ²²⁵AC- mAb no. 3-macropa, ²²⁵AC- mAb no. 3-Tet5 and ²²⁵AC acetate per gram sample ororgan.

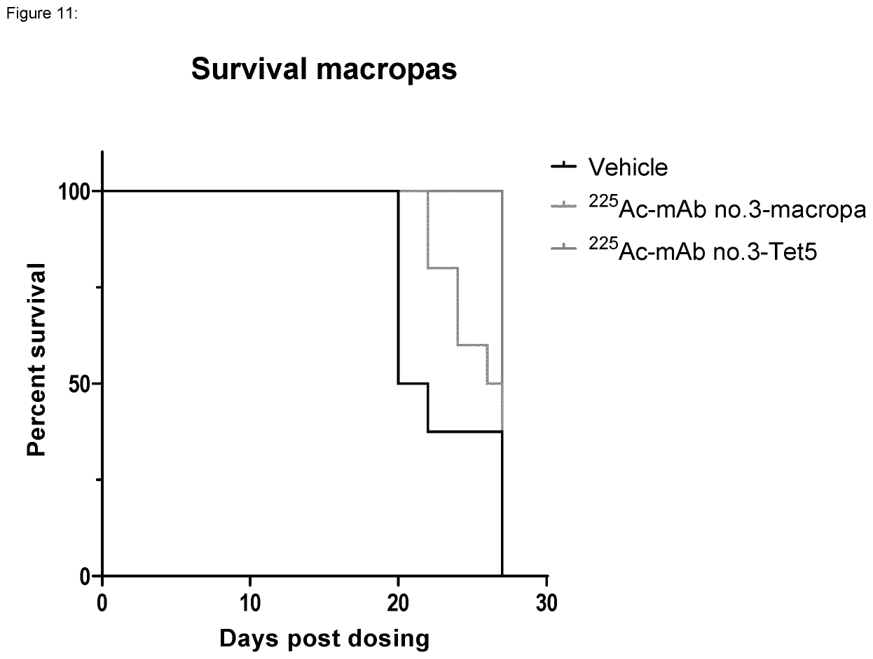

FIG. 11 illustrates survival plot HEP-3B treated mice after injection of ²²⁵Ac-mAb no. 3-macropa and ²²⁵Ac-mAb no. 3-Tet5

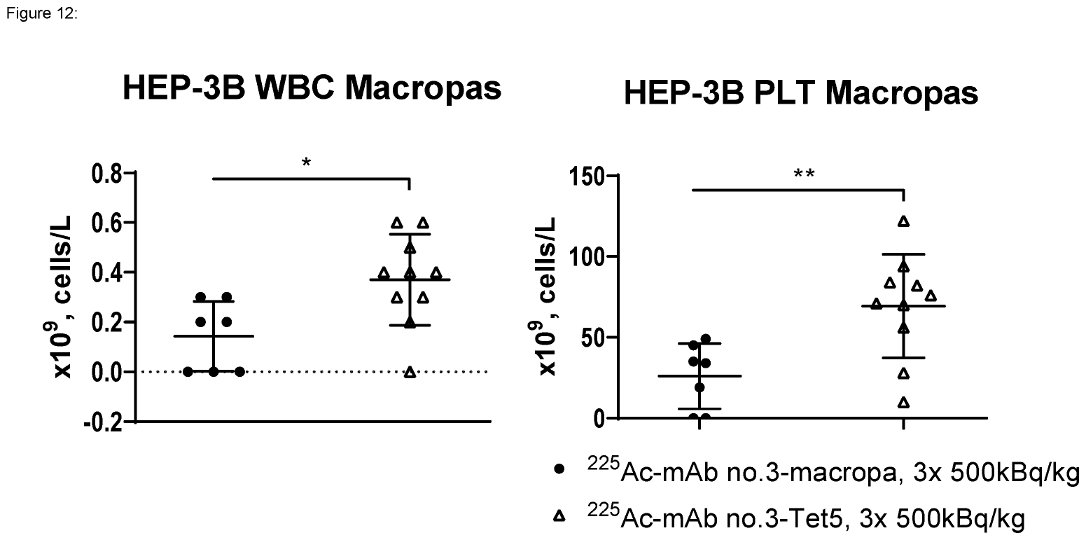

FIG. 12 illustrates white blood cell and platelets count for ²²⁵Ac-mAb no. 3-macropa and ²²⁵AC--mAb no. 3Tet5

An efficacy study of Ac-225 labelled mAb no. 3-macropa and mAb no. 3-Tet5 was conducted. The compounds were labeled with Ac-225 in 0.1 M acetate, pH 5, and injected respectively in HEP-3B treated mice three times at 500 kBq/kg at 7 days intervals. Saline was injected separately as vehicle control. Tumor sizes were measured at time points up to 28 days.

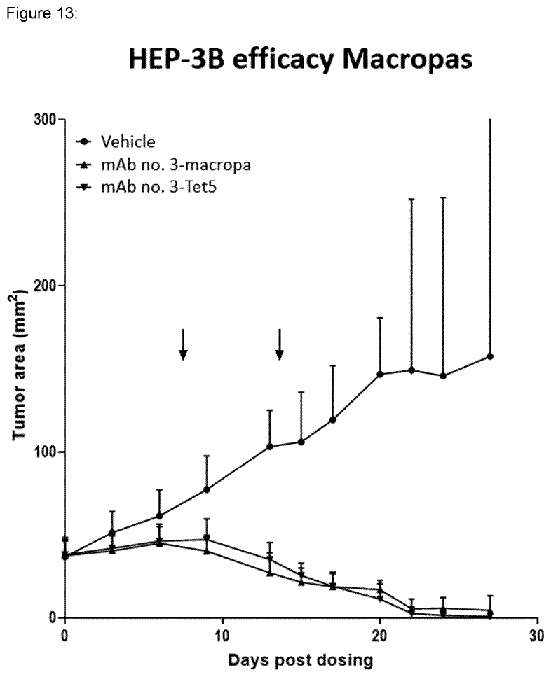

FIG. 13 illustrates tumor area for HEP-3B mice after treatment with ²²⁵Ac-mAb no. 3-macropa and ²²⁵Ac-mAb no. 3-Tet5

1. A compound of general formula (I):
[(C)n-L]-(V)m (I),
in which: C represents the macrocyclic chelating agent macropa, L represents a multi-functional linker moiety comprising multiple functional groups for the covalent attachment of C, and V is a tissue-targeting moiety, and wherein n is a natural number selected from 2 to 32 and m is from 1 to 5, or a stereoisomer, a tautomer, an N-oxide, a hydrate, a solvate, or a salt thereof, or a mixture of same.

2. The compound of claim 1, wherein the compound further comprises an alpha-emitting radioisotope or a stereoisomer, a tautomer, an N-oxide, a hydrate, a solvate, or a salt thereof, or a mixture of same.

3. The compound of claim 2, wherein the alpha-emitting radioisotope is selected from the group consisting of radium-223, radium-224, bismuth-212, bismuth-213 and actinium-225 or a stereoisomer, a tautomer, an N-oxide, a hydrate, a solvate, or a salt thereof, or a mixture of same.

4. The compound of claim 1, wherein the tissue-targeting moiety is a monoclonal antibody or a stereoisomer, a tautomer, an N-oxide, a hydrate, a solvate, or a salt thereof, or a mixture of same.

5. The compound of claim 1, wherein L is a multi-functional linker moiety comprising multiple functional groups for the covalent attachment of a chelator such as a polyamine or polyacid-containing backbone or amino acid containing polymer comprising side-chains with amino, thiol or carboxylic acid moieties such as lysine, cysteine or glutamic acid.

6. The compound of claim 1, wherein L is

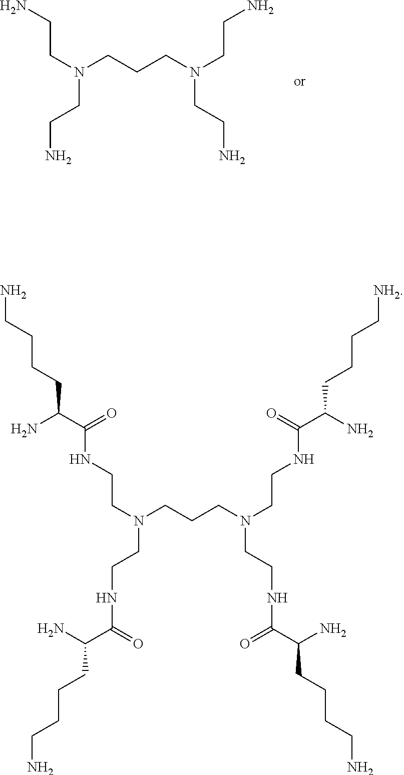 [SMILES: NCCN(CCN)CCCN(CCN)CCN ; NCCCC[C@H](N)C(=O)NCCN(CCCN(CCNC(=O)[C@@H](N)CCCCN)CCNC(=O)[C@@H](N)CCCCN)CCNC(=O)[C@@H](N)CCCCN]

7. The compound of claim 1, wherein C is the macrocyclic chelating agent macropa of formula (A) below: 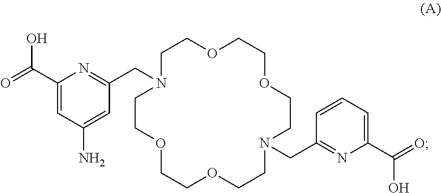 [SMILES: Nc1cc(CN2CCOCCOCCN(Cc3cccc(C(=O)O)n3)CCOCCOCC2)nc(C(=O)O)c1]
and
wherein either the amino substituent group or the carboxylic acid groups are used to form amide bonds with either L or V, n is 2, and V is a monoclonal antibody, or a stereoisomer, a tautomer, an N-oxide, a hydrate, a solvate, or a salt thereof, or a mixture of same.

8. The compound of claim 1, wherein C is the macrocyclic chelating agent macropa of formula (A) and wherein either the amino substituent group or the carboxylic acid groups are used to form amide bonds with either L or V, n is 3, and V is a monoclonal antibody, or a stereoisomer, a tautomer, an N-oxide, a hydrate, a solvate, or a salt thereof, or a mixture of same.

9. The compound of claim 1, wherein C is the macrocyclic chelating agent macropa of formula (A) and wherein either the amino substituent group or the carboxylic acid groups are used to form amide bonds with either L or V, n is 4, and V is a monoclonal antibody, or a stereoisomer, a tautomer, an N-oxide, a hydrate, a solvate, or a salt thereof, or a mixture of same.

10. The compound of claim 1, wherein C is the macrocyclic chelating agent macropa of formula (A) and wherein either the amino substituent group or the carboxylic acid groups are used to form amide bonds with either L or V, n is 8 and V is a monoclonal antibody, or a stereoisomer, a tautomer, an N-oxide, a hydrate, a solvate, or a salt thereof, or a mixture of same.

11. The compound of claim 1, wherein C is the macrocyclic chelating agent macropa of formula (B) below: 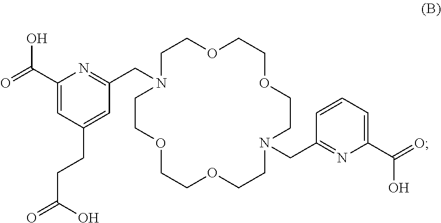 [SMILES: O=C(O)CCc1cc(CN2CCOCCOCCN(Cc3cccc(C(=O)O)n3)CCOCCOCC2)nc(C(=O)O)c1]
and
wherein the carboxylic acid groups are used to form amide bonds with either L or V, n is 2, and V is a monoclonal antibody, or a stereoisomer, a tautomer, an N-oxide, a hydrate, a solvate, or a salt thereof, or a mixture of same.

12. The compound of claim 1, wherein C is the macrocyclic chelating agent macropa of formula (B) and wherein the carboxylic acid groups are used to form amide bonds with either L or V, n is 3, and V is a monoclonal antibody, or a stereoisomer, a tautomer, an N-oxide, a hydrate, a solvate, or a salt thereof, or a mixture of same.

13. The compound of claim 1, wherein C is the macrocyclic chelating agent macropa of formula (B) and wherein the carboxylic acid groups are used to form amide bonds with either L or V, n is 4, and V is a monoclonal antibody, or a stereoisomer, a tautomer, an N-oxide, a hydrate, a solvate, or a salt thereof, or a mixture of same.

14. The compound of claim 1, wherein C is the macrocyclic chelating agent macropa of formula (B) and wherein the carboxylic acid groups are used to form amide bonds with either L or V, n is 8, and V is a monoclonal antibody, or a stereoisomer, a tautomer, an N-oxide, a hydrate, a solvate, or a salt thereof, or a mixture of same.

15. The compound of claim 1 wherein the compound is selected from the group consisting of:
4,4′-[(9,13-bis{2-[2-(2-{[2-carboxy-6-({16-[(6-carboxypyridin-2-yl)methyl]-1,4,10,13 -tetraoxa-7,16-diazacyclooctadecan-7-yl}methyl)pyridin-4-yl]amino}-2-oxoethoxy)acetamido]ethyl}-1,5,17,21-tetraoxo-3,19-dioxa-6,9,13,16-tetraazahenicosane-1,21-diyl)diimino]bis[6-({16-[(6-carboxypyridin-2-yl)methyl]-1,4,10,13-tetraoxa-7,16-diazacyclooctadecan-7-yl}methyl)pyridine-2-carboxylic acid] (Example 7; Tet2);
4,4′-[7,11-bis(2-{3-[2-carboxy-6-({16-[(6-carboxypyridin-2-yl)methyl]-1,4,10,13 -tetraoxa-7,16-diazacyclooctadecan-7-yl}methyl)pyridin-4-yl]propanamido}ethyl)-3,15-dioxo-4,7,11,14-tetraazaheptadecane-1,17-diyl]bis[6-({16-[(6-carboxypyridin-2-yl)methyl]-1,4,10,13-tetraoxa-7,16-diazacyclooctadecan-7-yl}methyl)pyridine-2-carboxylic acid] (Example 10, Tet5); and
4-[3-[[6-[2-[3-[bis[2-[2,6-bis[3-[2-carboxy-6-[[16-[(6-carboxy-2-pyridyl)methyl]-1,4,10,13-tetraoxa-7,16-diazacyclooctadec-7-yl]methyl]-4-pyridyl]propanoylamino]hexanoylamino]ethyl]amino]propyl-[2-[2,6-bis[3-[2-carboxy-6-[[16-[(6-carboxy-2-pyridyl)methyl]-1,4,10,13-tetraoxa-7,16-diazacyclooctadec-7-yl]methyl]-4-pyridyl]propanoylamino]hexanoylamino]ethyl]amino]ethylamino]-5-[3-[2-carboxy-6-[[16-[(6-carboxy-2-pyridyl)methyl]-1,4,10,13-tetraoxa-7,16-diazacyclooctadec-7-yl]methyl]-4-pyridyl]propanoylamino]-6-oxo-hexyl]amino]-3-oxo-propyl]-6-[[16-[(6-carboxy-2-pyridyl)methyl]-1,4,10,13-tetraoxa-7,16-diazacyclooctadec-7-yl]methyl]pyridine-2-carboxylic acid (Example 14, Oct2).

16. A method of preparing a compound of claim 1, or a stereoisomer, a tautomer, an N-oxide, a hydrate, a solvate, or a salt thereof, or a mixture of same, said method comprising: reacting an intermediate compound of general formula (II):
[(X)p′-C]n-L (II),
in which C, L, n and m and m are as defined for the compound of general formula (I) according to claim 1,
with V;
in which V is as defined for the compound of general formula (I) according to claim 1,
thereby giving a compound of general formula (I):
[(C)n-L]-(V)m (I),
in which C, L, V, n and and m are as defined for the compound of general formula (I) according to claim 1.

17. (canceled)

18. A pharmaceutical composition comprising a compound of claim 1, or a stereoisomer, a tautomer, an N-oxide, a hydrate, a solvate, or a salt thereof, or a mixture of same, and one or more pharmaceutically acceptable excipients.

19. A pharmaceutical combination comprising:
one or more first active ingredients, wherein the one or more first active ingredients comprises a compound of claim 1, or a stereoisomer, a tautomer, an N-oxide, a hydrate, a solvate, or a salt thereof, or a mixture of same, and
one or more further active ingredients.

20. A method for treatment or prophylaxis of a disease, the method comprising administering to a mammal in need thereof, an effective amount of a compound of claim 1, or a stereoisomer, a tautomer, an N-oxide, a hydrate, a solvate, or a salt thereof, or a mixture of same.

21. (canceled)

22. The method of claim 20, wherein the disease is a hyperproliferative disorder.

23. The method of claim 22, wherein the hyperproliferative disorder is an oncological disorder.

24. The pharmaceutical combination of claim 19, wherein the one or more further active ingredients comprises an anti-cancer agent.

25. The method of claim 20, wherein the mammal is a human.

26. A method for treatment or prophylaxis of a disease, the method comprising administering to a mammal in need thereof, an effective amount of a pharmaceutical composition of claim 18.

27. The method of claim 26, wherein the disease is a hyperproliferative disorder.

28. The method of claim 27, wherein the hyperproliferative disorder is an oncological disorder.

29. The method of claim 26, wherein the mammal is a human.

30. A method for treatment or prophylaxis of a disease, the method comprising administering to a mammal in need thereof, an effective amount of a pharmaceutical combination of claim 19.

31. The method of claim 30, wherein the disease is a hyperproliferative disorder.

32. The method of claim 31, wherein the hyperproliferative disorder is an oncological disorder.

33. The method of claim 30, wherein the mammal is a human.
